(19) United States
(12) Patent Application Publication
(10) Pub. No.: US 2021/0053957 A1
(43) Pub. Date: Feb. 25, 2021

(54) HERBICIDAL COMPOUNDS

(71) Applicant: SYNGENTA PARTICIPATIONS AG, Basel (CH)

(72) Inventors: James Nicholas SCUTT, Bracknell, Berkshire (GB); Nigel James WILLETTS, Bracknell, Berkshire (GB); Ravindra SONAWANE, Corlim, Ilhas Goa (IN); Mangala PHADTE, Corlim, Ilhas Goa (IN); Sandeep Reddy KANDUKURI, Corlim, Ilhas Goa (IN); Swarnendu SASMAL, Corlim, Ilhas Goa (IN); Sarah ARMSTRONG, Bracknell, Berkshire (GB); Andrea MCGRANAGHAN, Bracknell, Berkshire (GB)

(73) Assignee: SYNGENTA PARTICIPATIONS AG, Basel (CH)

(21) Appl. No.: 17/043,969

(22) PCT Filed: Mar. 29, 2019
(86) PCT No.: PCT/EP2019/058029

§ 371 Date: Sep. 30, 2020

(30) Foreign Application Priority Data
Mar. 30, 2018 (IN) 201811012074

(51) Int. Cl. C07D 413/04; C07D 417/04; C07D 403/04; A01N 43/82; A01N 43/76; A01N 43/78; A01N 43/58; C07D 405/04; C07D 409/04

(52) U.S. Cl. CPC C07D 413/04; C07D 417/04; C07D 403/04; A01N 43/82; C07D 409/04; A01N 43/78; A01N 43/58; C07D 405/04; A01N 43/76

(57) ABSTRACT

Compounds of the formula (I) wherein the substituents are as defined in claim 1, useful as pesticides, especially as herbicides.

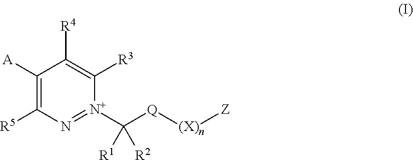

The present invention relates to herbicidally active pyridazine derivatives, as well as to processes and intermediates used for the preparation of such derivatives. The invention further extends to herbicidal compositions comprising such derivatives, as well as to the use of such compounds and compositions for controlling undesirable plant growth: in particular the use for controlling weeds, in crops of useful plants.

The present invention is based on the finding that pyridazine derivatives of formula (I) as defined herein, exhibit surprisingly good herbicidal activity. Thus, according to the present invention there is provided a compound of formula (I) or an agronomically acceptable salt or zwitterionic species thereof:

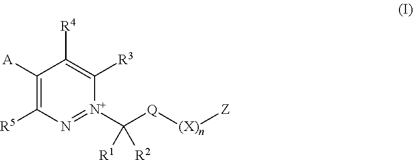

wherein
R¹ is selected from the group consisting of hydrogen, halogen, C₁-C₆alkyl, C₂-C₆alkenyl, C₂-C₆alkynyl, C₃-C₆cycloalkyl, C₁-C₆haloalkyl, —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵;
R² is selected from the group consisting of hydrogen, halogen, C₁-C₆alkyl and C₁-C₆haloalkyl;
and wherein when R¹ is selected from the group consisting of —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵, R² is selected from the group consisting of hydrogen and C₁-C₆alkyl; or
R¹ and R² together with the carbon atom to which they are attached form a C₃-C₆cycloalkyl ring or a 3- to 6-membered heterocyclyl, which comprises 1 or 2 heteroatoms individually selected from N and O; and
Q is (CR^{1a}R^{2b})ₘ;
m is 0, 1, 2 or 3;
each R^{1a} and R^{2b} are independently selected from the group consisting of hydrogen, halogen, C₁-C₆alkyl, C₁-C₆haloalkyl, —OH, —OR⁷, —OR^{15a}, —NH₂, —NHR⁷, —NHR^{15a}, —N(R⁶)CHO, —NR^{7b}R^{7c} and —S(O)ᵣR¹⁵; or
each R^{1a} and R^{2b} together with the carbon atom to which they are attached form a C₃-C₆cycloalkyl ring or a 3- to 6-membered heterocyclyl, which comprises 1 or 2 heteroatoms individually selected from N and O; and
R³, R⁴ and R⁵ are independently selected from the group consisting of hydrogen, halogen, cyano, nitro, —S(O)ᵣR¹⁵, C₁-C₆alkyl, C₁-C₆fluoroalkyl, C₁-C₆fluoroalkoxy, C₁-C₆alkoxy, C₃-C₆cycloalkyl and —N(R⁶)₂;
each R⁶ is independently selected from hydrogen and C₁-C₆alkyl;
each R⁷ is independently selected from the group consisting of C₁-C₆alkyl, —S(O)₂R¹⁵, —C(O)R¹⁵, —C(O)OR¹⁵ and —C(O)NR¹⁶R¹⁷;
each R^{7a} is independently selected from the group consisting of —S(O)₂R¹⁵, —C(O)R¹⁵, —C(O)OR¹⁵, —C(O)NR¹⁶R¹⁷ and —C(O)NR⁶R^{15a};
R^{7b} and R^{7c} are independently selected from the group consisting of C₁-C₆alkyl, —S(O)₂R¹⁵, —C(O)R¹⁵, —C(O)OR¹⁵, —C(O)NR¹⁶R¹⁷ and phenyl, and wherein said phenyl is optionally substituted by 1, 2 or 3 R⁹ substituents, which may be the same or different; or
R^{7b} and R^{7c} together with the nitrogen atom to which they are attached form a 4- to 6-membered heterocyclyl ring which optionally comprises one additional heteroatom individually selected from N, O and S; and
A is a 5-membered heteroaryl attached to the rest of the molecule via a ring carbon atom, which comprises 1, 2, 3 or 4 heteroatoms independently selected from the group consisting of N, O and S, and wherein the heteroaryl is optionally substituted by 1, 2 or 3 R⁸ substituents, which may be the same or different,
and wherein when A is substituted on one or more ring carbon atoms, each R⁸ is independently selected from the group consisting of halogen, nitro, cyano, —NH₂, —NHR⁷, —N(R⁷)₂, —OH, —OR⁷, —S(O)ᵣR¹⁵, —NR⁶S(O)₂R¹⁵, —C(O)OR¹⁰, —C(O)R¹⁵, —C(O)NR¹⁶R¹⁷, —S(O)₂NR¹⁶R¹⁷, C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₃alkoxyC₁-C₃alkoxy-, C₁-C₆haloalkoxy, C₁-C₃haloalkoxyC₁-C₃alkyl-, C₃-C₆alkenyloxy, C₃-C₆alkynyloxy, N—C₃-C₆cycloalkylamino, —C(R⁶)═NOR⁶, phenyl, a 3- to 6-membered heterocyclyl, which comprises 1 or 2 heteroatoms individually selected from N and O, and a 5- or 6-membered heteroaryl, which comprises 1, 2, 3 or 4 heteroatoms independently selected from N, O and S, and wherein said phenyl, heterocyclyl or heteroaryl are optionally substituted by 1, 2 or 3 R⁹ substituents, which may be the same or different;
and/or
when A is substituted on a ring nitrogen atom, R⁸ is selected from the group consisting of —OR⁷, C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₃alkyl-, C₁-C₃alkoxyC₁-C₃alkoxy-, C₁-C₆haloalkoxy, C₁-C₃haloalkoxyC₁-C₃alkyl-, C₃-C₆alkenyloxy and C₃-C₆alkynyloxy; and
each R⁹ is independently selected from the group consisting of halogen, cyano, —OH, —N(R⁶)₂, C₁-C₄alkyl, C₁-C₄alkoxy, C₁-C₄haloalkyl and C₁-C₄haloalkoxy;
X is independently selected from the group consisting of C₃-C₆cycloalkyl, phenyl, a 5- or 6-membered heteroaryl, which comprises 1, 2, 3 or 4 heteroatoms independently selected from N, O and S, and a 4- to 6-membered heterocyclyl, which comprises 1, 2 or 3 heteroatoms independently selected from N, O and S, and wherein said cycloalkyl, phenyl, heteroaryl or heterocyclyl moieties are optionally substituted by 1 or 2 substituents, which may be the same or different, selected from R⁹, and wherein the aforementioned CR¹R² and Z, or Q and Z, moieties may be attached at any position of said cycloalkyl, phenyl, heteroaryl or heterocyclyl moieties;
n is 0 or 1;
Z is selected from the group consisting of —C(O)OR¹⁰, —CH₂OH, —CHO, —C(O)NHOR¹¹, —C(O)NHCN, —OC(O)NHOR¹¹, —OC(O)NHCN, —NR⁶C(O)NHOR¹¹, —NR⁶C(O)NHCN, —C(O)NHS(O)₂R¹², —OC(O)NHS(O)₂R¹², —NR⁶C(O)NHS(O)₂R¹², —S(O)₂OR¹⁰, —OS(O)₂OR¹⁰, —NR⁶S(O)₂OR¹⁰, —NR⁶S(O)OR¹⁰, —NHS(O)₂R¹⁴, —S(O)OR¹⁰, —OS(O)OR¹⁰, —S(O)₂NHCN, —S(O)₂NHC(O)R¹⁸, —S(O)₂NHS(O)₂R¹², —OS(O)₂NHCN, —OS(O)₂NHS(O)₂R¹², —OS(O)₂NHC(O)R¹⁸, —NR⁶S(O)₂NHCN, —NR⁶S(O)₂NHC(O)R¹⁸, —N(OH)C(O)R¹⁵, —ONHC(O)R¹⁵, —NR⁶S(O)₂NHS(O)₂R¹², —P(O)(R¹³)(OR¹⁰), —P(O)H(OR¹⁰), —OP(O)(R¹³)(OR¹⁰), —NR⁶P(O)(R¹³)(OR¹⁰) and tetrazole;
R¹⁰ is selected from the group consisting of hydrogen, C₁-C₆alkyl, phenyl and benzyl, and wherein said phenyl or benzyl are optionally substituted by 1, 2 or 3 R⁹ substituents, which may be the same or different;
R¹¹ is selected from the group consisting of hydrogen, C₁-C₆alkyl and phenyl, and wherein said phenyl is optionally substituted by 1, 2 or 3 R⁹ substituents, which may be the same or different;
R¹² is selected from the group consisting of C₁-C₆alkyl, C₁-C₆haloalkyl, C₁-C₆alkoxy, —OH, —N(R⁶)₂ and phenyl, and wherein said phenyl is optionally substituted by 1, 2 or 3 R⁹ substituents, which may be the same or different;
R¹³ is selected from the group consisting of —OH, C₁-C₃alkyl, C₁-C₆alkoxy and phenyl;
R¹⁴ is C₁-C₆haloalkyl;
R¹⁵ is selected from the group consisting of C₁-C₆alkyl and phenyl, and wherein said phenyl is optionally substituted by 1, 2 or 3 R⁹ substituents, which may be the same or different;
R^{15a} is phenyl, wherein said phenyl is optionally substituted by 1, 2 or 3 R⁹ substituents, which may be the same or different;
R¹⁶ and R¹⁷ are independently selected from the group consisting of hydrogen and C₁-C₃alkyl; or
R¹⁶ and R¹⁷ together with the nitrogen atom to which they are attached form a 4- to 6-membered heterocyclyl ring which optionally comprises one additional heteroatom independently selected from N, O and S;
R¹⁸ is selected from the group consisting of hydrogen, C₁-C₆alkyl, C₁-C₆haloalkyl, C₁-C₆alkoxy, —N(R⁶)₂ and phenyl, and wherein said phenyl is optionally substituted by 1, 2 or 3 R⁹ substituents, which may be the same or different;
and
r is 0, 1 or 2.

According to a second aspect of the invention, there is provided a herbicidal composition comprising a herbicidally effective amount of a compound of formula (I) and an agrochemically-acceptable diluent or carrier. Such an agricultural composition may further comprise at least one additional active ingredient.

According to a third aspect of the invention, there is provided a method of controlling or preventing undesirable plant growth, wherein a herbicidally effective amount of a compound of formula (I), or a composition comprising this compound as active ingredient, is applied to the plants, to parts thereof or the locus thereof.

According to a fourth aspect of the invention, there is provided the use of a compound of formula (I) as a herbicide.

As used herein, the term “halogen” or “halo” refers to fluorine (fluoro), chlorine (chloro), bromine (bromo) or iodine (iodo), preferably fluorine, chlorine or bromine.

As used herein, cyano means a —CN group.

As used herein, hydroxy means an —OH group.

As used herein, nitro means an —NO₂ group.

As used herein, the term “C₁-C₆alkyl” refers to a straight or branched hydrocarbon chain radical consisting solely of carbon and hydrogen atoms, containing no unsaturation, having from one to six carbon atoms, and which is attached to the rest of the molecule by a single bond. C₁-C₄alkyl and C₁-C₂alkyl are to be construed accordingly. Examples of C₁-C₆alkyl include, but are not limited to, methyl (Me), ethyl (Et), n-propyl, 1-methylethyl (iso-propyl), n-butyl, and 1-dimethylethyl (t-butyl).

As used herein, the term “C₁-C₆alkoxy” refers to a radical of the formula —ORₐ where Rₐ is a C₁-C₆alkyl radical as generally defined above. C₁-C₄alkoxy is to be construed accordingly. Examples of C₁₋₄alkoxy include, but are not limited to, methoxy, ethoxy, propoxy, iso-propoxy and t-butoxy.

As used herein, the term “C₁-C₆haloalkyl” refers to a C₁-C₆alkyl radical, as generally defined above, substituted by one or more of the same or different halogen atoms. C₁-C₄haloalkyl is to be construed accordingly. Examples of C₁-C₆haloalkyl include, but are not limited to chloromethyl, fluoromethyl, fluoroethyl, difluoromethyl, trifluoromethyl and 2,2,2-trifluoroethyl.

As used herein, the term “C₁-C₆fluoroalkyl” refers to a C₁-C₆alkyl radical, as generally defined above, substituted by one or more fluorine atoms. Examples of C₁-C₆fluoroalkyl include, but are not limited to fluoromethyl, fluoroethyl, difluoromethyl, trifluoromethyl and 2,2,2-trifluoroethyl.

As used herein, the term “C₂-C₆alkenyl” refers to a straight or branched hydrocarbon chain radical consisting solely of carbon and hydrogen atoms, containing at least one double bond that can be of either the (E)- or (Z)-configuration, having from two to six carbon atoms, which is attached to the rest of the molecule by a single bond. C₂-C₄alkenyl is to be construed accordingly. Examples of C₂-C₆alkenyl include, but are not limited to, prop-1-enyl, allyl (prop-2-enyl) and but-1-enyl.

As used herein, the term “C₂-C₆haloalkenyl” refers to a C₂-C₆alkenyl radical, as generally defined above, substituted by one or more of the same or different halogen atoms. Examples of C₂-C₆haloalkenyl include, but are not limited to chloroethylene, fluoroethylene, 1,1-difluoroethylene, 1,1-dichloroethylene and 1,1,2-trichloroethylene.

As used herein, the term “C₂-C₆alkynyl” refers to a straight or branched hydrocarbon chain radical group consisting solely of carbon and hydrogen atoms, containing at least one triple bond, having from two to six carbon atoms, and which is attached to the rest of the molecule by a single bond. C₂-C₄alkynyl is to be construed accordingly. Examples of C₂-C₆alkynyl include, but are not limited to, prop-1-ynyl, propargyl (prop-2-ynyl) and but-1-ynyl.

As used herein, the term “C₁-C₆haloalkoxy” refers to a C₁-C₆alkoxy group, as defined above, substituted by one or more of the same or different halogen atoms. C₁-C₄haloalkoxy is to be construed accordingly. Examples of C₁-C₆haloalkoxy include, but are not limited to, fluoromethoxy, difluoromethoxy, fluoroethoxy, trifluoromethoxy and trifluoroethoxy.

As used herein, the term “C₁-C₆fluoroalkoxy” refers to a C₁-C₆alkoxy group, as defined above, substituted by one or more fluorine atoms. Examples of C₁-C₆fluoroalkoxy include, but are not limited to, fluoromethoxy, difluoromethoxy, fluoroethoxy, trifluoromethoxy and trifluoroethoxy.

As used herein, the term “C₁-C₃haloalkoxyC₁-C₃alkyl” refers to a radical of the formula R_{b}—O—Rₐ— where R_{b} is a C₁-C₆haloalkyl radical, as generally defined above, and Rₐ is a C₁-C₃alkylene radical as generally defined above.

As used herein, the term “C₁-C₃alkoxyC₁-C₃alkyl” refers to a radical of the formula R_{b}—O—Rₐ— where R_{b} is a C₁-C₃alkyl radical, as generally defined above, and Rₐ is a C₁-C₃alkylene radical as generally defined above.

As used herein, the term “C₁-C₃alkoxyC₁-C₃alkoxy-” refers to a radical of the formula R_{b}—O—Rₐ—O— where R_{b} is a C₁-C₃alkyl radical, as generally defined above, and Rₐ is a C₁-C₃alkylene radical as generally defined above.

As used herein, the term “C₃-C₆alkenyloxy” refers to a radical of the formula —ORₐ where Rₐ is a C₃-C₆alkenyl radical, as generally defined above.

As used herein, the term “C₃-C₆alkynyloxy” refers to a radical of the formula —ORₐ where Rₐ is a C₃-C₆alkynyl radical, as generally defined above.

As used herein, the term “hydroxyC₁-C₆alkyl” refers to a C₁-C₆alkyl radical, as generally defined above, substituted by one or more hydroxy groups.

As used herein, the term “C₃-C₆cycloalkyl” refers to a stable, monocyclic ring radical which is saturated or partially unsaturated and contains 3 to 6 carbon atoms. C₃-C₄cycloalkyl is to be construed accordingly. Examples of C₃-C₆cycloalkyl include, but are not limited to, cyclopropyl, cyclobutyl, cyclopentyl and cyclohexyl.

As used herein, the term “C₃-C₆halocycloalkyl” refers to a C₃-C₆cycloalkyl radical, as generally defined above, substituted by one or more of the same or different halogen atoms. C₃-C₄halocycloalkyl is to be construed accordingly.

As used herein, the term “C₃-C₆cycloalkoxy” refers to a radical of the formula —ORₐ where Rₐ is a C₃-C₆cycloalkyl radical as generally defined above.

As used herein, the term “N—C₃-C₆cycloalkylamino” refers to a radical of the formula —NHRₐ where Rₐ is a C₃-C₆cycloalkyl radical as generally defined above.

As used herein, except where explicitly stated otherwise, the term “heteroaryl” refers to a 5- or 6-membered monocyclic aromatic ring which comprises 1, 2, 3 or 4 heteroatoms individually selected from N, O and S. The heteroaryl radical may be attached to the rest of the molecule via a carbon atom or heteroatom. Examples of heteroaryl include, furyl, pyrrolyl, imidazolyl, thienyl, pyrazolyl, thiazolyl, isothiazolyl, oxazolyl, isoxazolyl, triazolyl, tetrazolyl, pyrazinyl, pyridazinyl, pyrimidyl or pyridyl.

As used herein, except where explicitly stated otherwise, the term “heterocyclyl” or “heterocyclic” refers to a stable 4- to 6-membered non-aromatic monocyclic ring radical which comprises 1, 2, or 3 heteroatoms individually selected from N, O and S. The heterocyclyl radical may be bonded to the rest of the molecule via a carbon atom or heteroatom. Examples of heterocyclyl include, but are not limited to, pyrrolinyl, pyrrolidyl, tetrahydrofuryl, tetrahydrothienyl, tetrahydrothiopyranyl, piperidyl, piperazinyl, tetrahydropyranyl, dihydroisoxazolyl, dioxolanyl, morpholinyl or δ-lactamyl.

The presence of one or more possible asymmetric carbon atoms in a compound of formula (I) means that the compounds may occur in chiral isomeric forms, i.e., enantiomeric or diastereomeric forms. Also atropisomers may occur as a result of restricted rotation about a single bond. A compound of formula (I) is intended to include all those possible isomeric forms and mixtures thereof. The present invention includes all those possible isomeric forms and mixtures thereof for a compound of formula (I).

Likewise, formula (I) is intended to include all possible tautomers (including lactam-lactim tautomerism and keto-enol tautomerism) where present. The present invention includes all possible tautomeric forms for a compound of formula (I). Similarly, where there are di-substituted alkenes, these may be present in E or Z form or as mixtures of both in any proportion. The present invention includes all these possible isomeric forms and mixtures thereof for a compound of formula (I).

The compounds of formula (I) will typically be provided in the form of an agronomically acceptable salt, a zwitterion or an agronomically acceptable salt of a zwitterion. This invention covers all such agronomically acceptable salts, zwitterions and mixtures thereof in all proportions.

For example a compound of formula (I) wherein Z comprises an acidic proton, may exist as a zwitterion, a compound of formula (I-I), or as an agronomically acceptable salt, a compound of formula (I-I) as shown below:

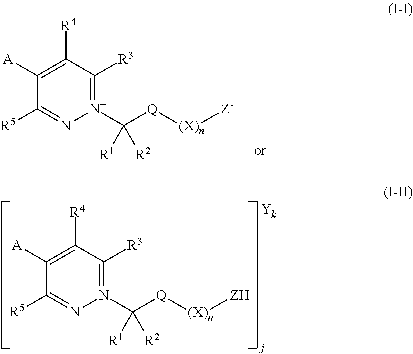

wherein, Y represents an agronomically acceptable anion and j and k represent integers that may be selected from 1, 2 or 3, dependent upon the charge of the respective anion Y.

A compound of formula (I) may also exist as an agronomically acceptable salt of a zwitterion, a compound of formula (I-II) as shown below:

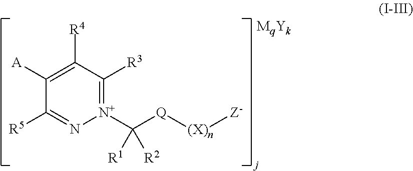

wherein, Y represents an agronomically acceptable anion, M represents an agronomically acceptable cation (in addition to the pyridazinium cation) and the integers j, k and q may be selected from 1, 2 or 3, dependent upon the charge of the respective anion Y and respective cation M.

Thus where a compound of formula (I) is drawn in protonated form herein, the skilled person would appreciate that it could equally be represented in unprotonated or salt form with one or more relevant counter ions.

In one embodiment of the invention there is provided a compound of formula (I-II) wherein k is 2, j is 1 and Y is selected from the group consisting of halogen, trifluoroacetate and pentafluoropropionate. In this embodiment a nitrogen atom in ring A may be protonated or a nitrogen atom comprised in R¹, R², Q or X may be protonated. Preferably, in a compound of formula (I-II), k is 2, j is 1 and Y is chloride, wherein a nitrogen atom in ring A is protonated (for example a pyrrole or imidazole nitrogen is protonated).

Suitable agronomically acceptable salts of the present invention, represented by an anion Y, include, but are not limited to; chloride, bromide, iodide, fluoride, 2-naphthalenesulfonate, acetate, adipate, methoxide, ethoxide, propoxide, butoxide, aspartate, benzenesulfonate, benzoate, bicarbonate, bisulfate, bitartrate, butylsulfate, butylsulfonate, butyrate, camphorate, camsylate, caprate, caproate, caprylate, carbonate, citrate, diphosphate, edetate, edisylate, enanthate, ethanedisulfonate, ethanesulfonate, ethylsulfate, formate, fumarate, gluceptate, gluconate, glucoronate, glutamate, glycerophosphate, heptadecanoate, hexadecanoate, hydrogen sulfate, hydroxide, hydroxynaphthoate, isethionate, lactate, lactobionate, laurate, malate, maleate, mandelate, mesylate, methanedisulfonate, methylsulfate, mucate, myristate, napsylate, nitrate, nonadecanoate, octadecanoate, oxalate, pelargonate, pentadecanoate, pentafluoropropionate, perchlorate, phosphate, propionate, propylsulfate, propylsulfonate, succinate, sulfate, tartrate, tosylate, tridecylate, triflate, trifluoroacetate, undecylinate and valerate.

Suitable cations represented by M include, but are not limited to, metals, conjugate acids of amines and organic cations. Examples of suitable metals include aluminium, calcium, cesium, copper, lithium, magnesium, manganese, potassium, sodium, iron and zinc. Examples of suitable amines include allylamine, ammonia, amylamine, arginine, benethamine, benzathine, butenyl-2-amine, butylamine, butylethanolamine, cyclohexylamine, decylamine, diamylamine, dibutylamine, diethanolamine, diethylamine, diethylenetriamine, diheptylamine, dihexylamine, diisoamylamine, diisopropylamine, dimethylamine, dioctylamine, dipropanolamine, dipropargylamine, dipropylamine, dodecylamine, ethanolamine, ethylamine, ethylbutylamine, ethylenediamine, ethylheptylamine, ethyloctylamine, ethylpropanolamine, heptadecylamine, heptylamine, hexadecylamine, hexenyl-2-amine, hexylamine, hexylheptylamine, hexyloctylamine, histidine, indoline, isoamylamine, isobutanolamine, isobutylamine, isopropanolamine, isopropylamine, lysine, meglumine, methoxyethylamine, methylamine, methylbutylamine, methylethylamine, methylhexylamine, methylisopropylamine, methylnonylamine, methyloctadecylamine, methylpentadecylamine, morpholine, N,N-diethylethanolamine, N-methylpiperazine, nonylamine, octadecylamine, octylamine, oleylamine, pentadecylamine, pentenyl-2-amine, phenoxyethylamine, picoline, piperazine, piperidine, propanolamine, propylamine, propylenediamine, pyridine, pyrrolidine, sec-butylamine, stearylamine, tallowamine, tetradecylamine, tributylamine, tridecylamine, trimethylamine, triheptylamine, trihexylamine, triisobutylamine, triisodecylamine, triisopropylamine, trimethylamine, tripentylamine, tripropylamine, tris(hydroxymethyl)aminomethane, and undecylamine. Examples of suitable organic cations include benzyltributylammonium, benzyltrimethylammonium, benzyltriphenylphosphonium, choline, tetrabutylammonium, tetrabutylphosphonium, tetraethylammonium, tetraethylphosphonium, tetramethylammonium, tetramethylphosphonium, tetrapropylammonium, tetrapropylphosphonium, tributylsulfonium, tributylsulfoxonium, triethylsulfonium, triethylsulfoxonium, trimethylsulfonium, trimethylsulfoxonium, tripropylsulfonium and tripropylsulfoxonium.

Preferred compounds of formula (I) wherein Z comprises an acidic proton, can be represented as either (1-1) or (1-11). For compounds of formula (I-I) emphasis is given to salts when Y is chloride, bromide, iodide, hydroxide, bicarbonate, acetate, pentafluoropropionate, triflate, trifluoroacetate, hydrogen sulfate, methylsulfate, tosylate and nitrate, wherein j and k are each independently 1 or 2. Preferably, Y is chloride, bromide, iodide, hydroxide, bicarbonate, acetate, trifluoroacetate, methylsulfate, tosylate and nitrate, wherein j and k are 1. For compounds of formula (I-II) emphasis is also given to salts when Y is carbonate and sulfate, wherein j is 2 and k is 1, and when Y is phosphate, wherein j is 3 and k is 1.

Where appropriate compounds of formula (I) may also be in the form of (and/or be used as) an N-oxide.

Compounds of formula (I) wherein m is 0 and n is 0 may be represented by a compound of formula (I-Ia) as shown below:

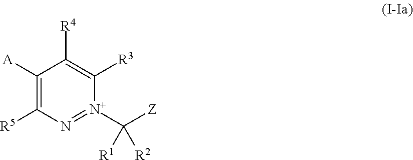

wherein R¹, R², R³, R⁴, R⁵, A and Z are as defined for compounds of formula (I).

Compounds of formula (I) wherein m is 1 and n is 0 may be represented by a compound of formula (I-Ib) as shown below:

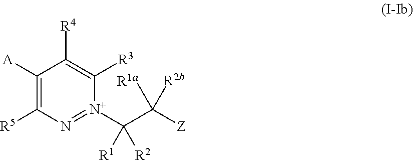

wherein R¹, R², R^{1a}, R^{2b}, R³, R⁴, R⁵, A and Z are as defined for compounds of formula (I).

Compounds of formula (I) wherein m is 2 and n is 0 may be represented by a compound of formula (I-Ic) as shown below:

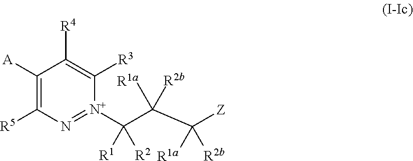

wherein R¹, R², R^{1a}, R^{2b}, R³, R⁴, R⁵, A and Z are as defined for compounds of formula (I).

Compounds of formula (I) wherein m is 3 and n is 0 may be represented by a compound of formula (I-Id) as shown below:

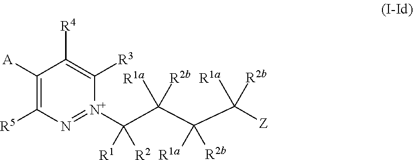

wherein R¹, R², R^{1a}, R^{2b}, R³, R⁴, R⁵, A and Z are as defined for compounds of formula (I).

The following list provides definitions, including preferred definitions, for substituents n, m, r, A, Q, X, Z, R¹, R², R, R^{2b}, R³, R⁴, R⁵, R⁶, R⁷, R^{7a}, R^{7b}, R^{7c}, R⁸, R^{8a}, R^{8b}, R^{8c}, R^{8d}, R⁹, R¹⁰, R¹¹, R¹², R¹³, R¹⁴, R¹⁵, R^{15a}, R¹⁶, R¹⁷ and R¹⁸ with reference to the compounds of formula (I) according to the invention. For any one of these substituents, any of the definitions given below may be combined with any definition of any other substituent given below or elsewhere in this document.

R¹ is selected from the group consisting of hydrogen, halogen, C₁-C₆alkyl, C₂-C₆alkenyl, C₂-C₆alkynyl, C₃-C₆cycloalkyl, C₁-C₆haloalkyl, —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵. Preferably, R¹ is selected from the group consisting of hydrogen, halogen, C₁-C₆alkyl, C₁-C₆fluoroalkyl, —OR⁷, —NHS(O)₂R¹⁵, —NHC(O)R¹⁵, —NHC(O)OR¹⁵, —NHC(O)NR¹⁶R¹⁷, —N(R^{7a})₂ and —S(O)ᵣR¹⁵. More preferably, R¹ is selected from the group consisting of hydrogen, halogen, C₁-C₆alkyl, C₁-C₆fluoroalkyl, —OR⁷ and —N(R^{7a})₂. Even more preferably, R¹ is selected from the group consisting of hydrogen, C₁-C₆alkyl, —OR and —N(R^{7a})₂. Even more preferably still, R¹ is hydrogen or C₁-C₆alkyl. Yet even more preferably still, R¹ is hydrogen or methyl. Most preferably R¹ is hydrogen.

R² is selected from the group consisting of hydrogen, halogen, C₁-C₆alkyl and C₁-C₆haloalkyl. Preferably, R² is selected from the group consisting of hydrogen, halogen, C₁-C₆alkyl and C₁-C₆fluoroalkyl. More preferably, R² is hydrogen or C₁-C₆alkyl. Even more preferably, R² is hydrogen or methyl. Most preferably R² is hydrogen.

Wherein when R¹ is selected from the group consisting of —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵, R² is selected from the group consisting of hydrogen and C₁-C₆alkyl. Preferably, when R¹ is selected from the group consisting of —OR⁷, —NHS(O)₂R¹⁵, —NHC(O)R¹⁵, —NHC(O)OR¹⁵, —NHC(O)NR¹⁶R¹⁷, —N(R^{7a})₂ and —S(O)ᵣR¹⁵, R² is selected from the group consisting of hydrogen and methyl.

Alternatively, R¹ and R² together with the carbon atom to which they are attached form a C₃-C₆cycloalkyl ring or a 3- to 6-membered heterocyclyl, which comprises 1 or 2 heteroatoms individually selected from N and O. Preferably, R¹ and R² together with the carbon atom to which they are attached form a C₃-C₆cycloalkyl ring. More preferably, R¹ and R² together with the carbon atom to which they are attached form a cyclopropyl ring.

In one embodiment R¹ and R² are hydrogen.

In another embodiment R¹ is methyl and R² is hydrogen.

In another embodiment R¹ is methyl and R² is methyl.

Q is (CR^{1a}R^{2b}).

m is 0, 1, 2 or 3. Preferably, m is 0, 1 or 2. More preferably, m is 1 or 2. Most preferably, m is 1.

Each R^{1a} and R^{2b} are independently selected from the group consisting of hydrogen, halogen, C₁-C₆alkyl, C₁-C₆haloalkyl, —OH, —OR⁷, —OR^{15a}, —NH₂, —NHR⁷, —NHR^{15a}, —N(R⁶)CHO, —NR^{7b}R^{7c}, and —S(O)ᵣR¹⁵. Preferably, each R^{1a} and R^{2b} are independently selected from the group consisting of hydrogen, halogen, C₁-C₆alkyl, C₁-C₆fluoroalkyl, —OH, —NH₂ and —NHR⁷. More preferably, each R^{1a} and R^{2b} are independently selected from the group consisting of hydrogen, C₁-C₆alkyl, —OH and —NH₂. Even more preferably, each R^{1a} and R^{2b} are independently selected from the group consisting of hydrogen, methyl, —OH and —NH₂. Even more preferably still, each R^{1a} and R^{2b} are independently selected from the group consisting of hydrogen and methyl. Most preferably R^{1a} and R^{2b} are hydrogen.

In another embodiment each R^{1a} and R^{2b} are independently selected from the group consisting of hydrogen and C₁-C₆alkyl.

Alternatively, each R^{1a} and R^{2b} together with the carbon atom to which they are attached form a C₃-C₆cycloalkyl ring or a 3- to 6-membered heterocyclyl, which comprises 1 or 2 heteroatoms individually selected from N and O. Preferably, each R^{1a} and R^{2b} together with the carbon atom to which they are attached form a C₃-C₆cycloalkyl ring. More preferably, each R^{1a} and R^{2b} together with the carbon atom to which they are attached form a cyclopropyl ring.

R³, R⁴ and R⁵ are independently selected from the group consisting of hydrogen, halogen, cyano, nitro, —S(O)ᵣR¹⁵, C₁-C₆alkyl, C₁-C₆fluoroalkyl, C₁-C₆fluoroalkoxy, C₁-C₆alkoxy, C₃-C₆cycloalkyl and —N(R⁶)₂. Preferably, R³, R⁴ and R⁵ are independently selected from the group consisting of hydrogen, C₁-C₆alkyl, C₁-C₆fluoroalkyl, C₁-C₆fluoroalkoxy, C₁-C₆alkoxy, C₃-C₆cycloalkyl and —N(R⁶)₂. More preferably, R³, R⁴ and R⁵ are independently selected from the group consisting of hydrogen, C₁-C₆alkyl and C₁-C₆alkoxy. Even more preferably, R³, R⁴ and R⁵ are independently selected from the group consisting of hydrogen and C₁-C₆alkyl. Even more preferably still, R³, R⁴ and R⁵ are independently selected from the group consisting of hydrogen and methyl. Most preferably, R³, R⁴ and R⁵ are hydrogen.

Each R⁶ is independently selected from hydrogen and C₁-C₆alkyl. Preferably, each R⁶ is independently selected from hydrogen and methyl.

Each R⁷ is independently selected from the group consisting of C₁-C₆alkyl, —S(O)₂R¹⁵, —C(O)R¹⁵, —C(O)OR¹⁵ and —C(O)NR¹⁶R¹⁷. Preferably, each R⁷ is independently selected from the group consisting of C₁-C₆alkyl, —C(O)R¹⁵ and —C(O)NR¹⁶R¹⁷. More preferably, each R⁷ is C₁-C₆alkyl. Most preferably, each R⁷ is methyl.

Each R^{7a} is independently selected from the group consisting of —S(O)₂R¹⁵, —C(O)R¹⁵, —C(O)OR¹⁵—C(O)NR¹⁶R¹⁷ and —C(O)NR⁶R^{1a}. Preferably, each R^{7a} is independently —C(O)R¹⁵ or —C(O)NR¹⁶R¹⁷.

R^{7b} and R^{7c} are independently selected from the group consisting of C₁-C₆alkyl, —S(O)₂R¹⁵, —C(O)R¹⁵, —C(O)OR¹⁵, —C(O)NR¹⁶R¹⁷ and phenyl, and wherein said phenyl is optionally substituted by 1, 2 or 3 R⁹ substituents, which may be the same or different. Preferably, R^{7b} and R^{7c} are independently selected from the group consisting of C₁-C₆alkyl, —C(O)R¹⁵ and —C(O)NR¹⁶R¹⁷. More preferably, R^{7b} and R^{7c} are C₁-C₆alkyl. Most preferably, R^{7b} and R^{7c} are methyl.

Alternatively, R^{7b} and R^{7c} together with the nitrogen atom to which they are attached form a 4- to 6-membered heterocyclyl ring which optionally comprises one additional heteroatom individually selected from N, O and S. Preferably, R^{7b} and R^{7c} together with the nitrogen atom to which they are attached form a 5- to 6-membered heterocyclyl ring which optionally comprises one additional heteroatom individually selected from N and O. More preferably, R^{7b} and R^{7c} together with the nitrogen atom to which they are attached form an pyrrolidyl, oxazolidinyl, imidazolidinyl, piperidyl, piperazinyl or morpholinyl group.

A is a 5-membered heteroaryl attached to the rest of the molecule via a ring carbon atom, which comprises 1, 2, 3 or 4 heteroatoms independently selected from the group consisting of N, O and S, and wherein the heteroaryl is optionally substituted by 1, 2 or 3 R⁸ substituents, which may be the same or different.

Preferably, A is a heteroaryl selected from the group consisting of 1,2,3,5-oxatriazolyl, 1,2,3,5-thiatriazolyl, 1,2,4-oxadiazolyl, 1,2,4-thiadiazolyl, 1,2,4-triazolyl, 1,2,5-oxadiazolyl, 1,2,5-thiadiazolyl, 1,3,4-oxadiazolyl, 1,3,4-thiadiazolyl, furyl, thienyl, imidazolyl, isothiazolyl, isoxazolyl, 1,2,3-oxadiazolyl, 1,2,3,4-oxatriazolyl, oxazolyl, pyrazolyl, pyrrolyl, tetrazolyl, 1,2,3-thiadiazolyl, 1,2,3,4-thiatriazolyl, thiazolyl and 1,2,3-triazolyl, wherein the heteroaryl is optionally substituted by 1, 2 or 3 R⁸ substituents, which may be the same or different.

More preferably, A is a heteroaryl selected from the group consisting of 1,2,3,5-oxatriazol-4-yl, 1,2,3,5-thiatriazol-4-yl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,2,4-triazol-5-yl, 1,2,5-oxadiazol-3-yl, 1,2,5-thiadiazol-3-yl, 1,3,4-oxadiazol-2-yl, 1,3,4-thiadiazol-2-yl, 2-furyl, 2-thienyl, 3-furyl, 3-thienyl, imidazol-2-yl, imidazol-4-yl, imidazol-5-yl, isothiazol-3-yl, isothiazol-4-yl, isothiazol-5-yl, isoxazol-3-yl, isoxazol-4-yl, isoxazol-5-yl, 1,2,3-oxadiazol-4-yl, 1,2,3-oxadiazol-5-yl, 1,2,3,4-oxatriazol-5-yl, oxazol-2-yl, oxazol-4-yl, oxazol-5-yl, pyrazol-3-yl, pyrazol-4-yl, pyrazol-5-yl, pyrrol-2-yl, pyrrol-3-yl, tetrazol-5-yl, 1,2,3-thiadiazol-4-yl, 1,2,3-thiadiazol-5-yl, 1,2,3,4-thiatriazol-5-yl, thiazol-2-yl, thiazol-4-yl, thiazol-5-yl, triazol-4-yl and triazol-5-yl wherein the heteroaryl is optionally substituted by 1, 2 or 3 R⁸ substituents, which may be the same or different.

In one embodiment, A is a heteroaryl selected from the group consisting of tetrazolyl, 1,2,4-triazolyl, isoxazolyl, oxazolyl, thiazolyl, 1,3,4-thiadiazolyl, 1,2,3-triazolyl, pyrazolyl, 1,3,4-oxadiazolyl, 1,2,4-thiadiazolyl, imidazolyl, isothiazolyl, thienyl, furyl, 1,2,4-oxadiazolyl, 1,2,3-thiadiazolyl and 1,2,5-thiadiazolyl, wherein the heteroaryl is optionally substituted by 1, 2 or 3 R⁸ substituents, which may be the same or different.

Even more preferably, A is a heteroaryl selected from the group consisting of tetrazol-5-yl, 1,2,4-triazol-3-yl, 1,2,4-triazol-5-yl, isoxazol-3-yl, oxazol-2-yl, thiazol-2-yl, 1,3,4-thiadiazol-2-yl, triazol-4-yl, triazol-5-yl, pyrazol-3-yl, pyrazol-5-yl, 1,3,4-oxadiazol-2-yl, 1,2,4-thiadiazol-5-yl, oxazol-4-yl, imidazol-2-yl, isothiazol-5-yl, 2-thienyl, 3-furyl, 2-furyl, isothiazol-4-yl, thiazol-4-yl, 3-thienyl, imidazol-5-yl, isoxazol-5-yl, 1,2,4-oxadiazol-5-yl, 1,2,4-thiadiazol-3-yl, isothiazol-3-yl, 1,2,3-thiadiazol-5-yl, 1,2,5-thiadiazol-3-yl, thiazol-5-yl and 1,2,3-thiadiazol-4-yl, wherein the heteroaryl is optionally substituted by 1, 2 or 3 R⁸ substituents, which may be the same or different.

Even more preferably, A is selected from the group consisting of formula A-I to A-XXXIV below

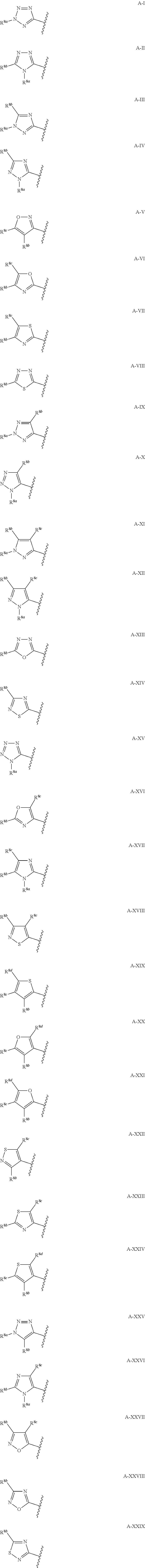

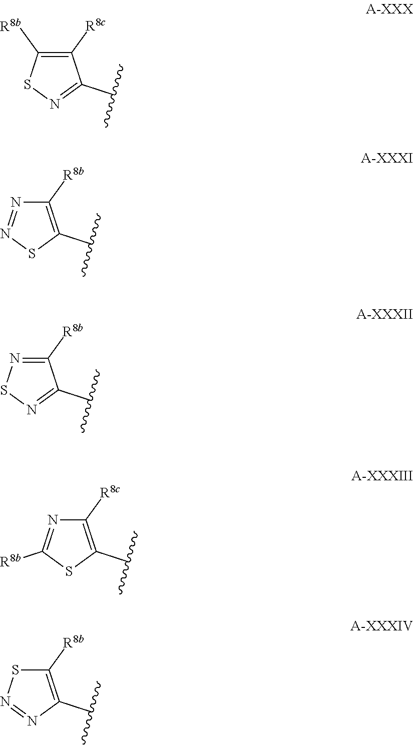

wherein the jagged line defines the point of attachment to the remaining part of a compound of formula (I), and R^{8a}, R^{8b}, R^{8c}, R^{8d} R⁶, R⁷, R¹⁰, R¹⁵, R¹⁶ and R¹⁷ are as defined herein. R^{8a}, R^{8b}, R^{8c}, R^{8d} are examples of R⁸ wherein the superscript letter a, b, c and d are used to denote positions within individual heterocycles (A-I to A-XXXIV).

Even more preferably still, A is selected from the group consisting of formula A-I to A-VIII, A-X, A-XIV, A-XVIII, A-XXVII, A-XXIX and A-XXX below

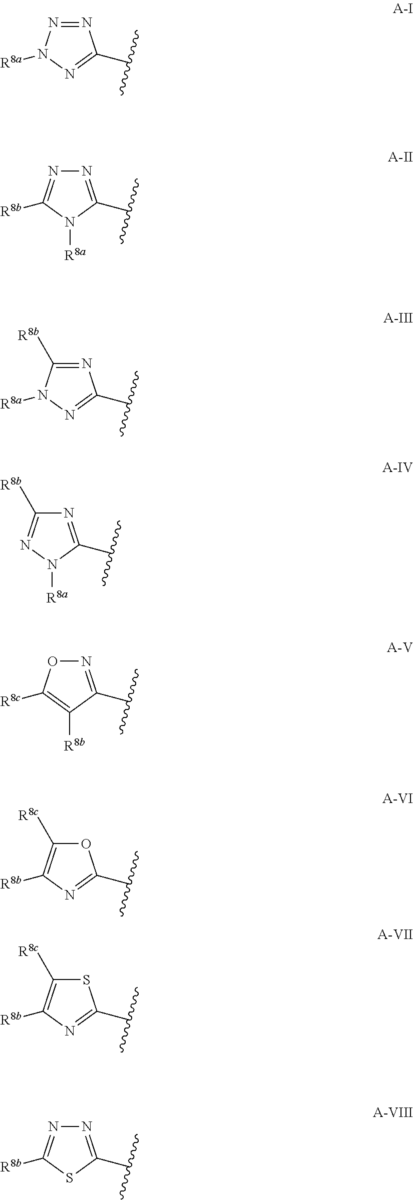

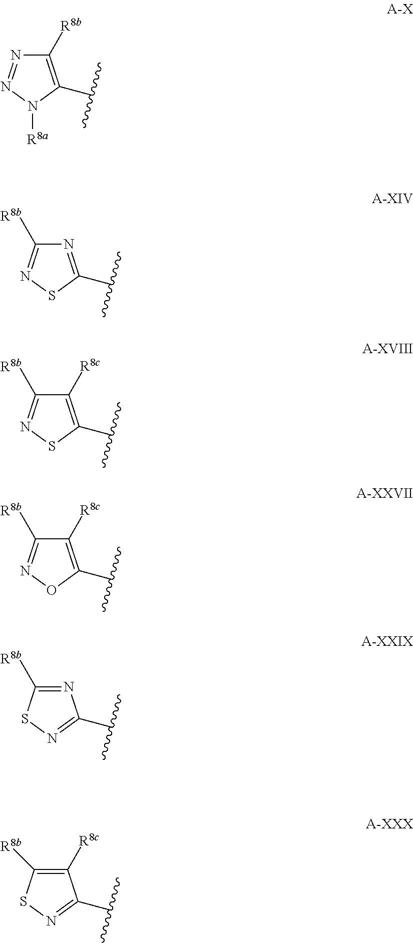

wherein the jagged line defines the point of attachment to the remaining part of a compound of formula (I) and R^{8a}, R^{8b}, R^{8c}, R^{8d} and R⁷ is as defined herein.

Yet, even more preferably still, A is selected from the group consisting of formula A-la, A-IIa, A-IIIa, A-IVa, A-Va, A-VIa, A-VIb, A-VIc, A-VIIa, A-VIIb, A-VIIIa, A-VIIb, A-Xa, A-XIVa, A-XVIIIa, A-XVIIIb, A-XXVIIa, A-XXIXa and A-XXXa below,

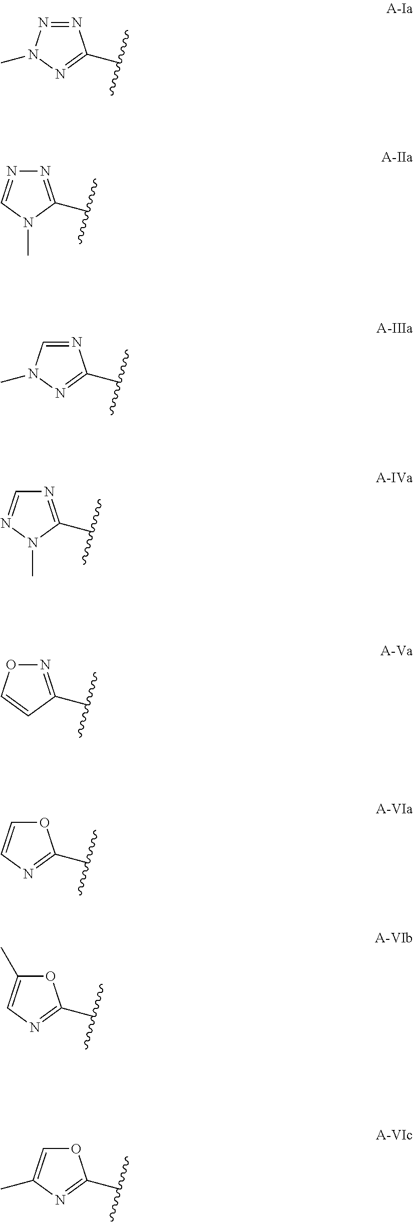

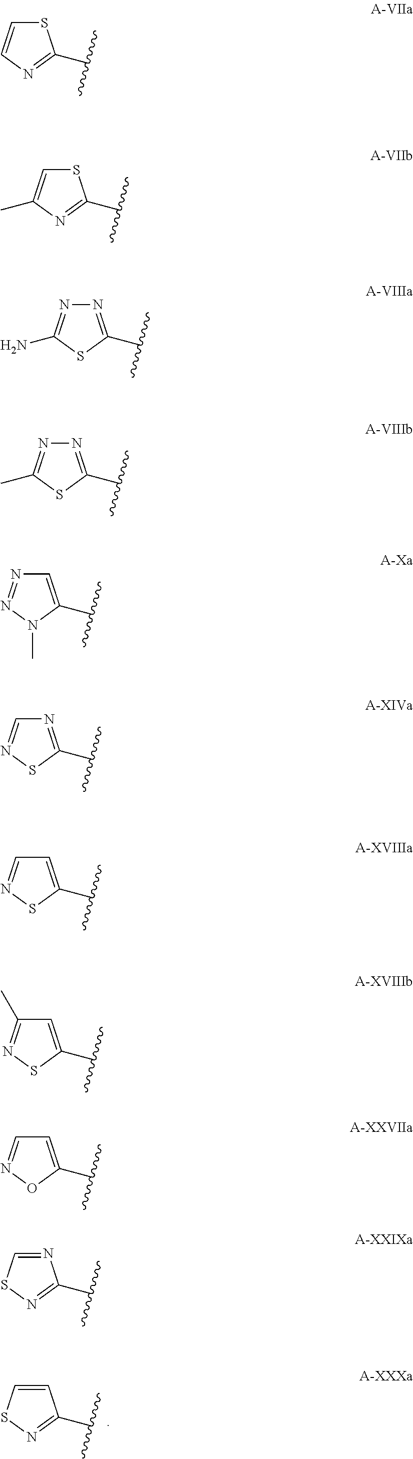

wherein the jagged line defines the point of attachment to the remaining part of a compound of formula (I).

In one embodiment, A is a heteroaryl selected from the group consisting of tetrazol-5-yl, 1,2,4-triazol-3-yl, 1,2,4-triazol-5-yl, isoxazol-3-yl, oxazol-2-yl, thiazol-2-yl, 1,3,4-thiadiazol-2-yl, triazol-4-yl, triazol-5-yl, pyrazol-3-yl, pyrazol-5-yl, 1,3,4-oxadiazol-2-yl, 1,2,4-thiadiazol-5-yl, oxazol-4-yl, imidazol-2-yl, isothiazol-5-yl, 2-thienyl, 3-furyl, 2-furyl, isothiazol-4-yl, thiazol-4-yl, 3-thienyl, imidazol-5-yl, isoxazol-5-yl and 1,2,4-oxadiazol-5-yl, wherein the heteroaryl may, where feasible, be optionally substituted by 1, 2 or 3 R⁸ substituents, which may be the same or different.

In another preferred embodiment, A is selected from the group consisting of formula A-I to A-XXVIII below

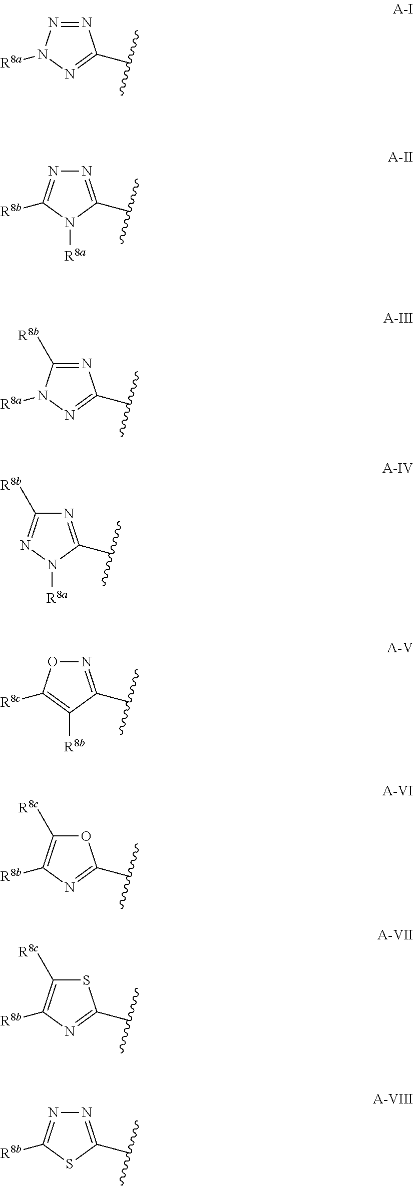

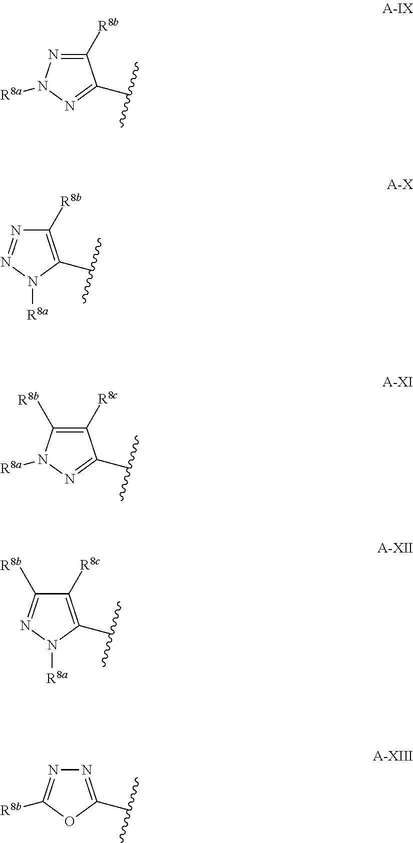

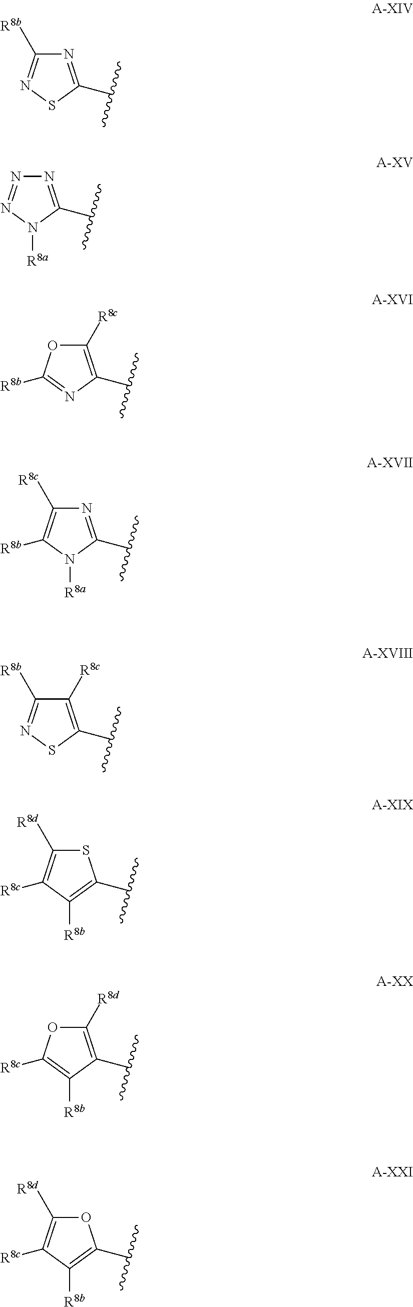

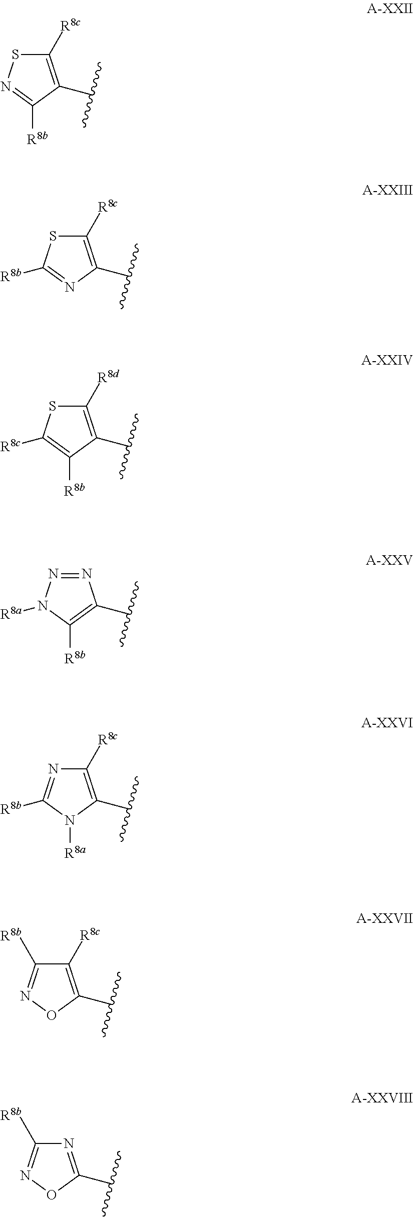

wherein the jagged line defines the point of attachment to the remaining part of a compound of formula (I), and R^{8a}, R^{8b}, R^{8c}, R^{8d} R⁶, R⁷, R¹⁰, R¹⁵, R¹⁶ and R¹⁷ are as defined herein. R^{8a}, R^{8b}, R^{8c}, R^{8d} are examples of R⁸ wherein the superscript letter a, b, c and d are used to denote positions within individual heterocycles (A-I to A-XXVIII).

In another more preferred embodiment, A is selected from the group consisting of formula A-I to A-VIII below

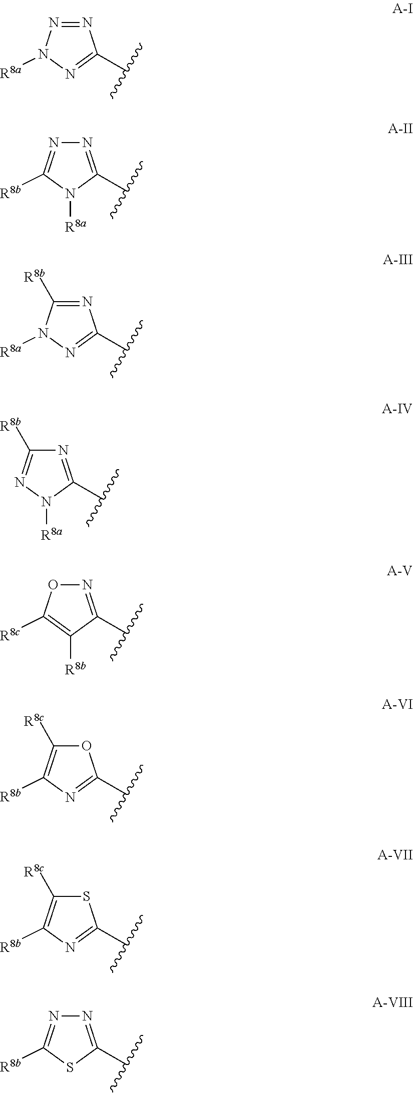

wherein the jagged line defines the point of attachment to the remaining part of a compound of formula (I) and R^{8a}, R^{8b}, R^{8c}, R^{8d} and R⁷ is as defined herein.

In an even more preferred embodiment, A is selected from the group consisting of formula A-la to A-VIIIa below

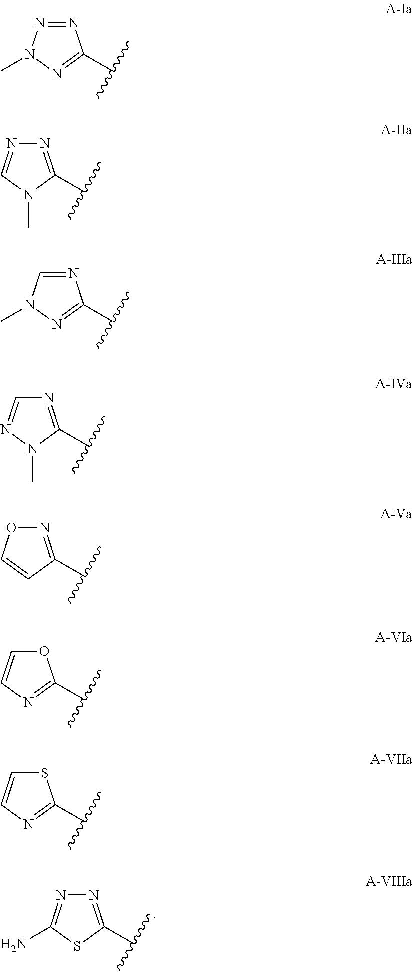

wherein the jagged line defines the point of attachment to the remaining part of a compound of formula (I).

When A is substituted on one or more ring carbon atoms, each R⁸ is independently selected from the group consisting of halogen, nitro, cyano, —NH₂, —NHR⁷, —N(R⁷)₂, —OH, —OR⁷, —S(O)ᵣR¹⁵, —NR⁶S(O)₂R¹⁵, —C(O)OR¹⁰, —C(O)R¹⁵, —C(O)NR¹⁶R¹⁷, —S(O)₂NR¹⁶R¹⁷, C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₃alkoxyC₁-C₃alkoxy-, C₁-C₆haloalkoxy, C₁-C₃haloalkoxyC₁-C₆alkyl-, C₃-C₆alkenyloxy, C₃-C₆alkynyloxy, N—C₃-C₆cycloalkylamino, —C(R⁶)═NOR⁶, phenyl, a 3- to 6-membered heterocyclyl, which comprises 1 or 2 heteroatoms individually selected from N and O, and a 5- or 6-membered heteroaryl, which comprises 1, 2, 3 or 4 heteroatoms independently selected from N, O and S, and wherein said phenyl, heterocyclyl or heteroaryl are optionally substituted by 1, 2 or 3 R⁹ substituents, which may be the same or different.

Preferably, when A is substituted on one or more ring carbon atoms, each R⁸ is independently selected from the group consisting of halogen, nitro, cyano, —NH₂, —NHR⁷, —N(R⁷)₂, —OH, —OR⁷, —S(O)ᵣR¹⁵, —NR⁶S(O)₂R¹⁵, —C(O)OR¹⁰, —C(O)R¹⁵, —C(O)NR¹⁶R¹⁷, —S(O)₂NR¹⁶R¹⁷, C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₃alkoxyC₁-C₃alkoxy-, C₁-C₆haloalkoxy, C₁-C₃haloalkoxyC₁-C₃alkyl-, C₃-C₆alkenyloxy, C₃-C₆alkynyloxy, —C(R⁶)═NOR⁶, phenyl and a 5- or 6-membered heteroaryl, which comprises 1, 2, 3 or 4 heteroatoms independently selected from N, O and S, and wherein said phenyl or heteroaryl are optionally substituted by 1, 2 or 3 R⁹ substituents, which may be the same or different.

More preferably, when A is substituted on one or more ring carbon atoms, each R⁸ is independently selected from the group consisting of halogen, nitro, cyano, —NH₂, —NHR⁷, —N(R⁷)₂, —OH, —OR⁷, —S(O)ᵣR¹⁵, —NR⁶S(O)₂R¹⁵, —C(O)OR¹⁰, —C(O)R¹⁵, —C(O)NR¹⁶R¹⁷, —S(O)₂NR¹⁶R¹⁷, C₁-C₆alkyl, C₁-C₆haloalkyl, C₃-C₆cycloalkyl, C₁-C₃alkoxyC₁-C₃alkyl-, hydroxyC₁-C₆alkyl-, C₁-C₃alkoxyC₁-C₃alkoxy-, C₁-C₆haloalkoxy, phenyl and a 6-membered heteroaryl, which comprises 1 or 2 nitrogen atoms, and wherein said phenyl or heteroaryl are optionally substituted by 1 or 2 R⁹ substituents, which may be the same or different.

Even more preferably, when A is substituted on one or more ring carbon atoms, each R⁸ is independently selected from the group consisting of halogen, nitro, cyano, —NH₂, —NHR⁷, —N(R⁷)₂, —OH, —OR⁷, —S(O)ᵣR¹⁵, —NR⁶S(O)₂R¹⁵, —C(O)OR¹⁰, —C(O)R¹⁵, —C(O)NR¹⁶R¹⁷, —S(O)₂NR¹⁶R¹⁷, C₁-C₆alkyl, C₁-C₆haloalkyl, C₃-C₆cycloalkyl, hydroxyC₁-C₆alkyl-, C₁-C₆haloalkoxy and a 6-membered heteroaryl, which comprises 1 or 2 nitrogen atoms, and wherein said heteroaryl is optionally substituted by 1 R⁹ substituent.

Even more preferably still, when A is substituted on one or more ring carbon atoms, each R⁸ is independently selected from the group consisting of halogen, cyano, —NH₂, —NHR⁷, —N(R⁷)₂, —OH, —OR⁷, —S(O)ᵣR¹⁵, —C(O)OR¹⁰, —C(O)R¹⁵, —C(O)NR¹⁶R¹⁷, C₁-C₆alkyl and C₁-C₆haloalkyl.

Further more preferably still, when A is substituted on one or more ring carbon atoms, each R⁸ is independently selected from the group consisting of chloro, fluoro, cyano, —NH₂, —NHMe, —N(Me)₂, —OH, —OMe, —S(O)₂Me, —C(O)OMe, —C(O)OEt, —C(O)OH, —C(O)Me, —C(O)NH₂, —C(O)NHMe, —C(O)N(Me)₂, methyl, iso-propyl and trifluoromethyl.

Yet further more preferably still, when A is substituted on one or more ring carbon atoms, each R⁸ is independently selected from the group consisting of chloro, cyano, —NH₂, —NHMe, —OMe, —C(O)OEt, —C(O)NHMe, methyl, iso-propyl and trifluoromethyl.

Most preferably, when A is substituted on one or more ring carbon atoms, each R⁸ is independently selected from the group consisting of chloro, —NH₂, —NHMe, —OMe, methyl, iso-propyl and trifluoromethyl.

When A is substituted on a ring nitrogen atom, R⁸ is selected from the group consisting of —OR, C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₆alkoxyC₁-C₃alkoxy-, C₁-C₆haloalkoxy, C₁-C₃haloalkoxyC₁-C₆alkyl-, C₃-C₆alkenyloxy and C₃-C₆alkynyloxy. Preferably, R⁸ is selected from the group consisting of —OR⁷, C₁-C₆alkyl and C₁-C₆haloalkyl. More preferably, R⁸ is —OR⁷ or C₁-C₆alky. Even more preferably still, R⁸ is C₁-C₆alky. Most preferably R⁸ is methyl.

When A is selected from the group consisting of formula A-I to A-XXXIV, R^{8a} (substituted on a ring nitrogen atom) is selected from the group consisting of hydrogen, C₁-C₆alkyl and C₁-C₆haloalkyl, and each R^{8b}, R^{8c} and R^{8d} (substituted on a ring carbon atom) are independently selected from the group consisting of hydrogen, halogen, nitro, cyano, —NH₂, —NHR⁷, —N(R⁷)₂, —OH, —OR⁷, —S(O)ᵣR¹⁵, —NR⁶S(O)₂R¹⁵, —C(O)OR¹⁰, —C(O)R¹⁵, —C(O)NR¹⁶R¹⁷, —S(O)₂NR¹⁶R¹⁷, C₁-C₆alkyl and C₁-C₆haloalkyl. Preferably R^{8a} is hydrogen or C₁-C₆alkyl and each R^{8b}, R^{8c} and R^{8d} are independently selected from the group consisting of hydrogen, halogen, cyano, —NH₂, —NHR⁷, —N(R⁷)₂, —OH, —OR⁷, —S(O)ᵣR¹⁵, —C(O)OR¹⁰, —C(O)R¹⁵, —C(O)NR¹⁶R¹⁷, C₁-C₆alkyl and C₁-C₆haloalkyl. More preferably, R^{8a} is hydrogen or methyl and each R^{8b}, R^{8c} and R^{8d} are independently selected from the group consisting of hydrogen, chloro, cyano, —NH₂, —NHMe, —OMe, —C(O)OEt, —C(O)NHMe, methyl, iso-propyl and trifluoromethyl. Even more preferably, R^{8a} is hydrogen or methyl and each R^{8b}, R^{8c} and R^{8d} are independently selected from the group consisting of hydrogen, chloro, —NH₂, —NHMe, —OMe, methyl, iso-propyl and trifluoromethyl.

In one embodiment, when A is selected from the group consisting of formula A-I to A-XXVIII, R^{8a} (substituted on a ring nitrogen atom) is selected from the group consisting of hydrogen, C₁-C₆alkyl and C₁-C₆haloalkyl, and each R^{8b}, R^{8c} and R^{8d} (substituted on a ring carbon atom) are independently selected from the group consisting of hydrogen, halogen, nitro, cyano, —NH₂, —NHR⁷, —N(R⁷)₂, —OH, —OR⁷, —S(O)ᵣR¹⁵, —NR⁶S(O)₂R¹⁵, —C(O)OR¹⁰, —C(O)R¹⁵, —C(O)NR¹⁶R¹⁷, —S(O)₂NR¹⁶R¹⁷, C₁-C₆alkyl and C₁-C₆haloalkyl. Preferably R^{8a} is hydrogen or C₁-C₆alkyl and each R^{8b}, R^{8c} and R^{8d} are independently selected from the group consisting of hydrogen, halogen, —NH₂, —NHR⁷, —N(R⁷)₂, —OR⁷, C₁-C₆alkyl and C₁-C₆haloalkyl. More preferably, R^{8a} is hydrogen or methyl and each R^{8b}, R^{8c} and R^{8d} are independently selected from the group consisting of hydrogen, chloro, —NH₂, —NHMe, —OMe, methyl, iso-propyl and trifluoromethyl.

When A is selected from the group consisting of formula A-I to A-VIII, A-X, A-XIV, A-XVIII, A-XXVII, A-XXIX and A-XXX, R^{8a} (substituted on a ring nitrogen atom) is hydrogen or C₁-C₃alkyl, and each R^{8b}, R^{8c} and R^{8d} (substituted on a ring carbon atom) are independently selected from the group consisting of hydrogen, halogen, cyano, —NH₂, —NHR⁷, —N(R⁷)₂, —OH, —OR⁷, —S(O)ᵣR¹⁵, —C(O)OR¹⁰, —C(O)R¹⁵, —C(O)NR¹⁶R¹⁷, C₁-C₆alkyl and C₁-C₆haloalkyl. Preferably, R^{8a} is hydrogen or methyl and each R^{8b}, R^{8c} and R^{8d} are independently selected from the group consisting of hydrogen, chloro, cyano, —NH₂, —NHMe, —OMe, —C(O)OEt, —C(O)NHMe, methyl, iso-propyl and trifluoromethyl. Even more preferably, R^{8a} is hydrogen or methyl and each R^{8b}, R^{8c} and R^{d} are independently selected from the group consisting of hydrogen, chloro, —NH₂, —NHMe, —OMe, methyl, iso-propyl and trifluoromethyl.

In one embodiment when A is selected from the group consisting of formula A-I to A-VIII, R^{8a} (substituted on a ring nitrogen atom) is hydrogen or C₁-C₃alkyl, and each R^{8b}, R^{8c} and R^{8d} (substituted on a ring carbon atom) are independently selected from the group consisting of hydrogen, halogen, —NH₂, —NHR⁷, —N(R⁷)₂, —OR⁷, C₁-C₆alkyl and C₁-C₆haloalkyl. Preferably, R^{8a} is hydrogen or methyl and each R^{8b}, R^{8c} and R^{8d} are independently selected from the group consisting of hydrogen, chloro, —NH₂, —NHMe, —OMe, methyl, iso-propyl and trifluoromethyl.

Each R⁹ is independently selected from the group consisting of halogen, cyano, —OH, —N(R⁶)₂, C₁-C₄alkyl, C₁-C₄alkoxy, C₁-C₄haloalkyl and C₁-C₄haloalkoxy. Preferably, each R⁹ is independently selected from the group consisting of halogen, cyano, —N(R⁶)₂, C₁-C₄alkyl, C₁-C₄alkoxy, C₁-C₄haloalkyl and C₁-C₄haloalkoxy. More preferably, each R⁹ is independently selected from the group consisting of halogen, C₁-C₄alkyl, C₁-C₄alkoxy and C₁-C₄haloalkyl. Even more preferably, each R⁹ is independently selected from the group consisting of halogen and C₁-C₄alkyl.

X is independently selected from the group consisting of C₃-C₆cycloalkyl, phenyl, a 5- or 6-membered heteroaryl, which comprises 1, 2, 3 or 4 heteroatoms independently selected from N, O and S, and a 4- to 6-membered heterocyclyl, which comprises 1, 2 or 3 heteroatoms independently selected from N, O and S, and wherein said cycloalkyl, phenyl, heteroaryl or heterocyclyl moieties are optionally substituted by 1 or 2 substituents, which may be the same or different, selected from R⁹, and wherein the aforementioned CR¹R² and Z, or Q and Z, moieties may be attached at any position of said cycloalkyl, phenyl, heteroaryl or heterocyclyl moieties.

Preferably X is independently selected from the group consisting of phenyl and a 4- to 6-membered heterocyclyl, which comprises 1 or 2 heteroatoms independently selected from N and O, and wherein said phenyl or heterocyclyl moieties are optionally substituted by 1 or 2 substituents, which may be the same or different, selected from R⁹, and wherein the aforementioned CR¹R², Q and Z moieties may be attached at any position of said heterocyclyl or phenyl moieties.

More preferably, X is independently selected from the group consisting of phenyl and a 5-membered heterocyclyl, which comprises 1 heteroatom, wherein said heteroatom is N, and wherein said phenyl or heterocyclyl moieties are optionally substituted by 1 or 2 substituents, which may be the same or different, selected from R⁹, and wherein the aforementioned CR¹R², Q and Z moieties may be attached at any position of said heterocyclyl or phenyl moieties.

Even more preferably, X is a 5-membered heterocyclyl, which comprises 1 heteroatom, wherein said heteroatom is N, and wherein the aforementioned CR¹R² and Q moieties are attached adjacent to the N atom and the Z moiety is attached to the N atom, or X is phenyl and the aforementioned CR¹R² and Q moieties are attached in a position ortho or meta to the Z moiety.

In one embodiment X is a 4- to 6-membered heterocyclyl, which comprises 1 or 2 heteroatoms independently selected from N and O, and wherein said heterocyclyl moieties is optionally substituted by 1 or 2 substituents, which may be the same or different, selected from R⁹.

n is 0 or 1. Preferably, n is 0.

Z is selected from the group consisting of —C(O)OR¹⁰, —CH₂OH, —CHO, —C(O)NHOR¹¹, —C(O)NHCN, —OC(O)NHOR¹¹, —OC(O)NHCN, —NR⁶C(O)NHOR¹¹, —NR⁶C(O)NHCN, —C(O)NHS(O)₂R¹², —OC(O)NHS(O)₂R¹², —NR⁶C(O)NHS(O)₂R¹², —S(O)₂OR¹⁰, —OS(O)₂OR¹⁰, —NR⁶S(O)₂OR¹⁰, —NR⁶S(O)OR¹⁰, —NHS(O)₂R¹⁴, —S(O)OR¹⁰, —OS(O)OR¹⁰, —S(O)₂NHCN, —S(O)₂NHC(O)R¹⁸, —S(O)₂NHS(O)₂R¹², —OS(O)₂NHCN, —OS(O)₂NHS(O)₂R¹², —OS(O)₂NHC(O)R¹⁸, —NR⁶S(O)₂NHCN, —NR⁶S(O)₂NHC(O)R¹⁸, —N(OH)C(O)R¹⁵, —ONHC(O)R¹⁵, —NR⁶S(O)₂NHS(O)₂R¹², —P(O)(R¹³)(OR¹⁰), —P(O)H(OR¹⁰), —OP(O)(R¹³)(OR¹⁰), —NR⁶P(O)(R¹³)(OR¹⁰) and tetrazole.

Preferably, Z is selected from the group consisting of —C(O)OR¹⁰, —C(O)NHOR¹¹, —OC(O)NHOR¹¹, —NR⁶C(O)NHOR¹¹, —C(O)NHS(O)₂R¹², —OC(O)NHS(O)₂R¹², —NR⁶C(O)NHS(O)₂R¹², —S(O)₂OR¹⁰, —OS(O)₂OR¹⁰, —NR⁶S(O)₂OR¹⁰, —NR⁶S(O)OR¹⁰, —NHS(O)₂R¹⁴, —S(O)OR¹⁰, —OS(O)OR¹⁰, —S(O)₂NHC(O)R¹⁸, —S(O)₂NHS(O)₂R¹², —OS(O)₂NHS(O)₂R¹², —OS(O)₂NHC(O)R¹⁸, —NR⁶S(O)₂NHC(O)R¹⁸, —N(OH)C(O)R¹⁵, —ONHC(O)R¹⁵, —NR⁶S(O)₂NHS(O)₂R¹², —P(O)(R¹³)(OR¹⁰), —P(O)H(OR¹⁰), —OP(O)(R¹³)(OR¹⁰) and —NR⁶P(O)(R¹³)(OR¹⁰).

More preferably, Z is selected from the group consisting of —C(O)OR¹⁰, —C(O)NHOR¹¹, —C(O)NHS(O)₂R¹², —S(O)₂OR¹⁰, —OS(O)₂OR¹⁰, —NR⁶S(O)₂OR¹⁰, —NHS(O)₂R¹⁴, —S(O)OR¹⁰ and —P(O)(R¹³)(OR¹⁰).

Even more preferably, Z is selected from the group consisting of —C(O)OR¹⁰, —C(O)NHS(O)₂R¹², —S(O)₂OR¹⁰, —OS(O)₂OR¹⁰ and —P(O)(R³)(OR¹⁰).

Even more preferably still, Z is selected from the group consisting of —C(O)OR¹⁰, —S(O)₂OR¹⁰, and —OS(O)₂OR¹⁰.

Yet even more preferably still, Z is selected from the group consisting of —C(O)OH, —C(O)OCH₂CH₃, —S(O)₂OH, —S(O)₂OCH₂C(CH₃)₃ and —OS(O)₂OH.

Most preferably, Z is —C(O)OH or —S(O)₂OH.

In one embodiment Z is selected from the group consisting of —C(O)OR¹⁰, —CH₂OH, —C(O)NHS(O)₂R¹², —S(O)₂OR¹⁰, —OS(O)₂OR¹⁰, —NR⁶S(O)₂R¹⁰ and —P(O)(R¹³)(OR¹⁰). Preferably, Z is selected from the group consisting of —C(O)OH, —C(O)OH₃, —C(O)OCH₂CH₃, —CH₂OH, —C(O)NHS(O)₂CH₃, —S(O)₂OH, —S(O)₂OCH₂C(CH₃)₃, —OS(O)₂OH, —NHS(O)₂OH, —P(O)(OH)(OH), —P(O)(OCH₃)(OCH₃), —P(O)(OH)(OCH₃), —P(O)(OH)(OCH₂CH₃) and —P(O)(OCH₂CH₃)(OCH₂CH₃).

R¹⁰ is selected from the group consisting of hydrogen, C₁-C₆alkyl, phenyl and benzyl, and wherein said phenyl or benzyl are optionally substituted by 1, 2 or 3 R⁹ substituents, which may be the same or different. Preferably, R¹ is selected from the group consisting of hydrogen, C₁-C₆alkyl, phenyl and benzyl. More preferably, R¹⁰ is selected from the group consisting of hydrogen and C₁-C₃alkyl. Most preferably, R¹⁰ is hydrogen.

R¹¹ is selected from the group consisting of hydrogen, C₁-C₆alkyl and phenyl, and wherein said phenyl is optionally substituted by 1, 2 or 3 R⁹ substituents, which may be the same or different. Preferably, R¹¹ is selected from the group consisting of hydrogen, C₁-C₃alkyl and phenyl. More preferably, R¹¹ is selected from the group consisting of hydrogen and C₁-C₃alkyl. Even more preferably, R¹¹ is C₁-C₆alkyl. Most preferably, R¹¹ is methyl.

R¹² is selected from the group consisting of C₁-C₃alkyl, C₁-C₆haloalkyl, C₁-C₆alkoxy, —OH, —N(R⁶)₂ and phenyl, and wherein said phenyl is optionally substituted by 1, 2 or 3 R⁹ substituents, which may be the same or different. Preferably, R¹² is selected from the group consisting of C₁-C₆alkyl, C₁-C₆haloalkyl, C₁-C₆alkoxy, —OH, —N(R⁶)₂ and phenyl. More preferably, R¹² is selected from the group consisting of C₁-C₆alkyl, C₁-C₆haloalkyl and —N(R⁶)₂. Even more preferably, R¹² is selected from the group consisting of methyl, —N(Me)₂ and trifluoromethyl. Most preferably, R¹² is methyl.

R¹³ is selected from the group consisting of —OH, C₁-C₆alkyl, C₁-C₆alkoxy and phenyl. Preferably R¹³ is selected from the group consisting of —OH, C₁-C₆alkyl and C₁-C₆alkoxy. More preferably, R¹³ is selected from the group consisting of —OH and C₁-C₆alkoxy. Even more preferably, R¹³ is selected from the group consisting of —OH, methoxy and ethoxy. Most preferably, R¹³ is —OH.

R¹⁴ is C₁-C₆haloalkyl. Preferably, R¹⁴ is trifluoromethyl.

R¹⁵ is selected from the group consisting of C₁-C₃alkyl and phenyl, and wherein said phenyl is optionally substituted by 1, 2 or 3 R⁹ substituents, which may be the same or different. Preferably, R¹⁵ is selected from the group consisting of C₁-C₆alkyl and phenyl. More preferably, R¹⁵ is C₁-C₆alkyl. Most preferably R¹⁵ is methyl.

R^{15a} is phenyl, wherein said phenyl is optionally substituted by 1, 2 or 3 R⁹ substituents, which may be the same or different. Preferably, R^{15a} is phenyl optionally substituted by 1 R⁹ substituent. More preferably, R^{15a} is phenyl.

R¹⁶ and R¹⁷ are independently selected from the group consisting of hydrogen and C₁-C₃alkyl. Preferably, R¹⁶ and R¹⁷ are independently selected from the group consisting of hydrogen and methyl.

Alternatively, R¹⁶ and R¹⁷ together with the nitrogen atom to which they are attached form a 4- to 6-membered heterocyclyl ring which optionally comprises one additional heteroatom independently selected from N, O and S. Preferably, R¹⁶ and R¹⁷ together with the nitrogen atom to which they are attached form a 5- to 6-membered heterocyclyl ring which optionally comprises one additional heteroatom independently selected from N and O. More preferably, R¹⁶ and R¹⁷ together with the nitrogen atom to which they are attached form an pyrrolidyl, oxazolidinyl, imidazolidinyl, piperidyl, piperazinyl or morpholinyl group.

R¹⁸ is selected from the group consisting of hydrogen, C₁-C₆alkyl, C₁-C₆haloalkyl, C₁-C₆alkoxy, —N(R⁶)₂ and phenyl, and wherein said phenyl is optionally substituted by 1, 2 or 3 R⁹ substituents, which may be the same or different. Preferably, R¹⁸ is selected from the group consisting of hydrogen, C₁-C₃alkyl, C₁-C₆haloalkyl, C₁-C₆alkoxy, —N(R⁶)₂ and phenyl. More preferably, R¹⁸ is selected from the group consisting of hydrogen, C₁-C₆alkyl and C₁-C₆haloalkyl. Further more preferably, R¹⁸ is selected from the group consisting of C₁-C₆alkyl and C₁-C₆haloalkyl. Most preferably, R¹⁸ is methyl or trifluoromethyl.

r is 0, 1 or 2. Preferably, r is 0 or 2.

In a set of preferred embodiments, in a compound according to formula (I) of the invention,

R¹ is hydrogen or C₁-C₆alkyl;

R² is hydrogen or methyl;
Q is (CR^{1a}R^{2b})ₘ;
m is 0, 1 or 2;
R^{1a} and R^{2b} are independently selected from the group consisting of hydrogen, C₁-C₆alkyl, —OH and —NH₂;
R³, R⁴ and R⁵ are independently selected from the group consisting of hydrogen and C₁-C₃alkyl;
each R⁶ is independently selected from hydrogen and methyl;
each R⁷ is C₁-C₆alkyl;
A is a 5-membered heteroaryl attached to the rest of the molecule via a ring carbon atom, which comprises 1, 2, 3 or 4 heteroatoms independently selected from the group consisting of N, O and S, and wherein the heteroaryl may, where feasible, be optionally substituted by 1, 2 or 3 R⁸ substituents, which may be the same or different;

when A is substituted on one or more ring carbon atoms, each R⁸ is independently selected from the group consisting of halogen, nitro, cyano, —NH₂, —NHR⁷, —N(R⁷)₂, —OH, —OR⁷, —S(O)ᵣR¹⁵, —NR⁶S(O)₂R¹⁵, —C(O)OR¹⁰, —C(O)R¹⁵, —C(O)NR¹⁶R¹⁷, —S(O)₂NR¹⁶R¹⁷, C₁-C₆alkyl and C₁-C₆haloalkyl;

and/or
when A is substituted on a ring nitrogen atom, R⁸ is selected from the group consisting of —OR⁷, C₁-C₆alkyl and C₁-C₆haloalkyl; and
n is 0;
Z is selected from the group consisting of —C(O)OR¹⁰, —C(O)NHS(O)₂R¹², —S(O)₂OR¹⁰, —OS(O)₂OR¹⁰ and —P(O)(R¹³)(OR¹⁰);
R¹⁰ is selected from the group consisting of hydrogen, C₁-C₆alkyl, phenyl and benzyl;
R¹² is selected from the group consisting of C₁-C₆alkyl, C₁-C₆haloalkyl and —N(R⁶)₂;
R¹³ is selected from the group consisting of —OH and C₁-C₆alkoxy;
R¹⁵ is C₁-C₆alkyl;
R¹⁶ and R¹⁷ are independently selected from the group consisting of hydrogen and methyl; and r is 0 or 2.

More preferably,

R¹ is hydrogen or methyl;

R² is hydrogen or methyl;
Q is (CR^{1a}R^{2b})ₘ;
m is 1 or 2;
R^{1a} and R^{2b} are independently selected from the group consisting of hydrogen and methyl;
R³, R⁴ and R⁵ are independently selected from the group consisting of hydrogen and methyl;
A is a heteroaryl selected from the group consisting of tetrazol-5-yl, 1,2,4-triazol-3-yl, 1,2,4-triazol-5-yl, isoxazol-3-yl, oxazol-2-yl, thiazol-2-yl, 1,3,4-thiadiazol-2-yl, triazol-4-yl, triazol-5-yl, pyrazol-3-yl, pyrazol-5-yl, 1,3,4-oxadiazol-2-yl, 1,2,4-thiadiazol-5-yl, oxazol-4-yl, imidazol-2-yl, isothiazol-5-yl, 2-thienyl, 3-furyl, 2-furyl, isothiazol-4-yl, thiazol-4-yl, 3-thienyl, imidazol-5-yl, isoxazol-5-yl and 1,2,4-oxadiazol-5-yl wherein the heteroaryl may, where feasible, be optionally substituted by 1, 2 or 3 R⁸ substituents, which may be the same or different;
when A is substituted on one or more ring carbon atoms, each R⁸ is independently selected from the group consisting of chloro, —NH₂, —NHMe, —OMe, methyl, iso-propyl and trifluoromethyl;
and/or
when A is substituted on a ring nitrogen atom, R⁸ is C₁-C₆alkyl; and
n is 0; and
Z is selected from the group consisting of —C(O)OR¹⁰, —S(O)₂OR¹⁰, and —OS(O)₂OR¹⁰;
R¹⁰ is hydrogen or C₁-C₆alkyl.

In another more preferred set of preferred embodiments, in a compound according to formula (I) of the invention,

R¹ is hydrogen or methyl;

R² is hydrogen or methyl;

Q is (CR^{1a}R^{2b})ₘ;

m is 1 or 2;
R^{1a} and R^{2b} are independently selected from the group consisting of hydrogen and methyl;
R³, R⁴ and R⁵ are independently selected from the group consisting of hydrogen and methyl;
each R⁶ is independently selected from hydrogen and methyl;
each R⁷ is C₁-C₆alkyl;
A is a heteroaryl selected from the group consisting of tetrazolyl, 1,2,4-triazolyl, isoxazolyl, oxazolyl, thiazolyl, 1,3,4-thiadiazolyl, 1,2,3-triazolyl, pyrazolyl, 1,3,4-oxadiazolyl, 1,2,4-thiadiazolyl, imidazolyl, isothiazolyl, thienyl, furyl, 1,2,4-oxadiazolyl, 1,2,3-thiadiazolyl and 1,2,5-thiadiazolyl, wherein the heteroaryl is optionally substituted by 1, 2 or 3 R⁸ substituents, which may be the same or different;
when A is substituted on one or more ring carbon atoms, each R⁸ is independently selected from the group consisting of halogen, cyano, —NH₂, —NHR⁷, —N(R⁷)₂, —OH, —OR⁷, —S(O)ᵣR¹⁵, —C(O)OR¹⁰, —C(O)R¹⁵, —C(O)NR¹⁶R¹⁷, C₁-C₆alkyl and C₁-C₆haloalkyl;
and/or
when A is substituted on a ring nitrogen atom, R⁸ is C₁-C₆alkyl; and
n is 0; and
Z is selected from the group consisting of —C(O)OR¹⁰, —CH₂OH, —C(O)NHS(O)₂R¹², —S(O)₂OR¹, —OS(O)₂OR¹⁰, —NR⁶S(O)₂OR¹⁰ and —P(O)(R¹³)(OR¹⁰);
R¹⁰ is hydrogen or C₁-C₆alkyl;
R¹² is selected from the group consisting of C₁-C₆alkyl, C₁-C₆haloalkyl and —N(R⁶)₂; and
R¹³ is selected from the group consisting of —OH and C₁-C₆alkoxy;
R¹⁵ is C₁-C₆alkyl;
R¹⁶ and R¹⁷ are independently selected from the group consisting of hydrogen and methyl; and
r is 0 or 2.

In a further set of preferred embodiments, the compound according to formula (I) is selected from the group consisting of a compound of formula (I-a), (I-b), (I-c), (I-d), (I-e), (I-f), (I-g), (I-h), (I-j), (I-k), (I-m), (I-n), (I-p), (I-q), (I-r), (I-s), (I-t), (I-u), (I-v), (I-w), (I-x), (I-y), (I-z), (I-a′), (I-b′), (I-c′), (I-d′) and (I-e′) below,

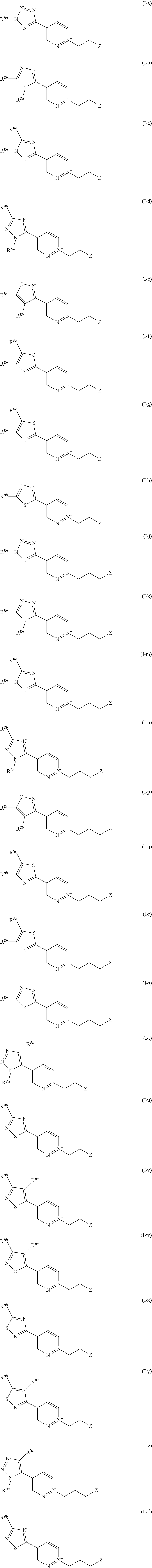

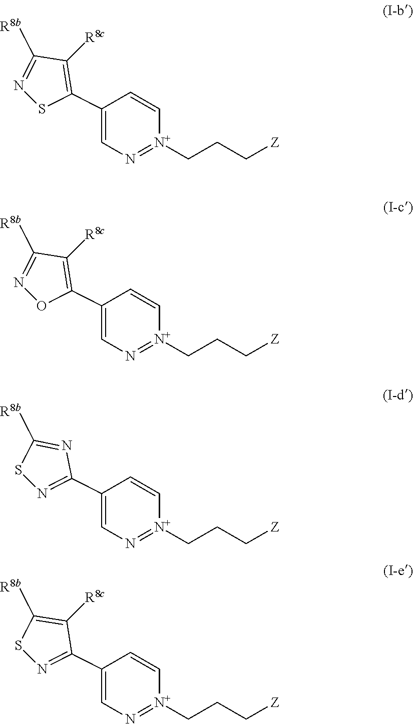

wherein in a compound of formula (I-a), (I-b), (I-c), (I-d), (I-e), (I-f), (I-g), (I-h), (I-j), (I-k), (I-m), (I-n), (I-p), (I-q), (I-r), (I-s) (I-t), (I-u), (I-v), (I-w), (I-x), (I-y), (I-z), (I-a′), (I-b′), (I-c′), (I-d′) and (I-e′);
R^{8a} is hydrogen or C₁-C₆alkyl;
each R^{8b}, R^{8c} and R^{8d} are independently selected from the group consisting of hydrogen, chloro, cyano, —NH₂, —NHMe, —OMe, —C(O)OEt, —C(O)NHMe, methyl, iso-propyl and trifluoromethyl; and
Z is selected from the group consisting of —C(O)OH, —C(O)OCH₃, —C(O)OCH₂CH₃, —CH₂OH, —C(O)NHS(O)₂CH₃, —S(O)₂OH, —S(O)₂OCH₂C(CH₃)₃, —OS(O)₂OH, —NHS(O)₂OH, —P(O)(OH)(OH), —P(O)(OCH₃)(OCH₃), —P(O)(OH)(OCH₃), —P(O)(OH)(OCH₂CH₃) and —P(O)(OCH₂CH₃)(OCH₂CH₃).

In another set of preferred embodiments, the compound according to formula (I) is selected from a compound of formula (I-a), (I-b), (I-c), (I-d), (I-e), (I-f), (I-g), (I-h), (I-j), (I-k), (I-m), (I-n), (I-p), (I-q), (I-r) or (I-s),

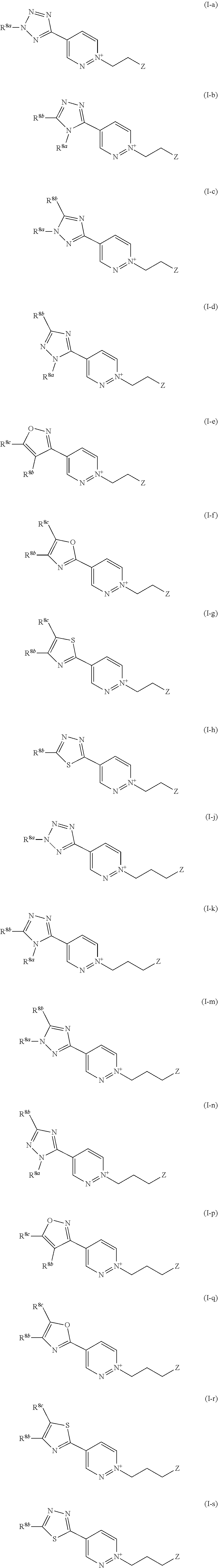

wherein in a compound of formula (I-a), (I-b), (I-c), (I-d), (I-e), (I-f), (I-g), (I-h), (I-j), (I-k), (I-m), (I-n), (I-p), (I-q), (I-r) or (I-s),

R^{8a} is hydrogen or C₁-C₆alkyl;

each R^{8b}, R^{8c} and R^{8d} are independently selected from the group consisting of hydrogen, halogen, —NH₂, —NHR⁷, —N(R⁷)₂, —OR⁷, C₁-C₆alkyl and C₁-C₆haloalkyl; and

### Z is —C(O)OH or —S(O)₂OH.

In a further more preferred set of embodiments, the compound according to formula (I) is selected from the group consisting of a compound of formula (I-aa), (I-bb), (I-cc), (I-dd), (I-ee), (I-ff), (I-gg), (I-hh), (I-jj), (I-kk), (I-mm), (I-nn), (I-pp), (I-qq), (I-rr), (I-ss), (I-tt), (I-uu), (I-vv), (I-ww), (I-xx), (I-yy), (I-zz), (I-aa′), (I-bb′), (I-cc′), (I-dd′), (I-ee′), (I-ff′), (I-gg′), (I-hh′), (I-j), (I-kk′), (I-mm′), (I-nn′), (I-pp′), (I-qq′) and (I-rr′) below,

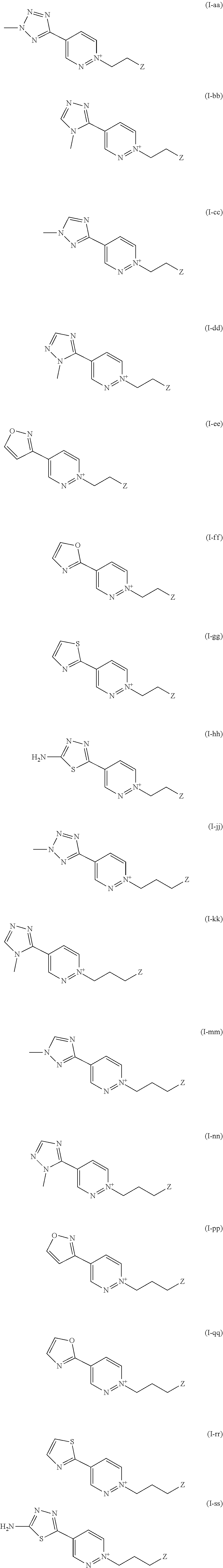

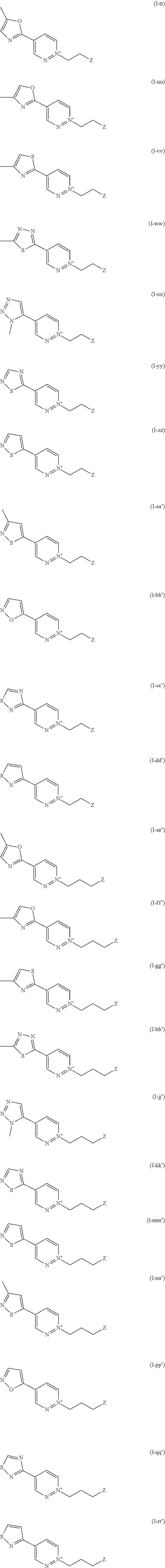

wherein in a compound of formula (I-aa), (I-bb), (I-cc), (I-dd), (I-ee), (I-ff), (I-gg), (I-hh), (I-jj), (I-kk), (I-mm), (I-nn), (I-pp), (I-qq), (I-rr), (I-ss), (I-tt), (I-uu), (I-vv), (I-ww), (I-xx), (I-yy), (I-zz), (I-aa′), (I-bb′), (I-cc′), (I-dd′), (I-ee′), (I-ff′), (I-gg′), (I-hh′), (I-jj′), (I-kk′), (I-mm′), (I-nn′), (I-pp′), (I-qq′) and (I-rr′);

### Z is —C(O)OH or —S(O)₂OH.

In another further more preferred set of embodiments, the compound according to formula (I) is selected from a compound of formula (I-aa), (I-bb), (I-cc), (I-dd), (I-ee), (I-ff), (I-gg), (I-hh), (I-jj), (I-kk), (I-mm), (I-nn), (I-pp), (I-qq), (I-rr) or (I-ss),

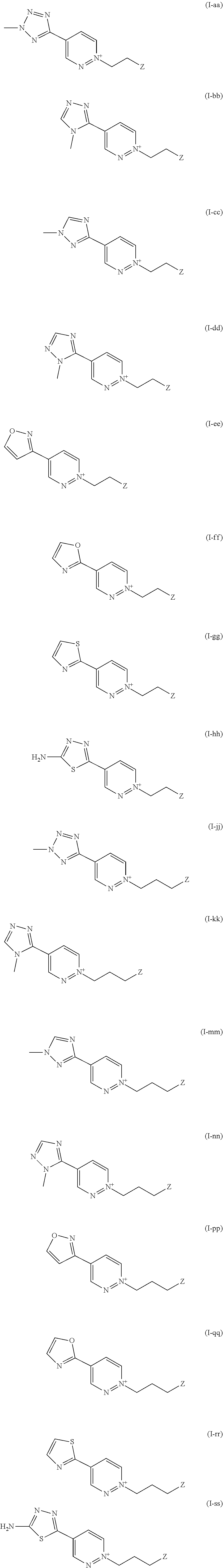

wherein in a compound of formula (I-aa), (I-bb), (I-cc), (I-dd), (I-ee), (I-ff), (I-gg), (I-hh), (I-jj), (I-kk), (I-mm), (I-nn), (I-pp), (I-qq), (I-rr) or (I-ss),

Z is —C(O)OH or —S(O)₂OH.

In one set of embodiments, the compound according to formula (I) is selected from a compound A1 to A147 listed in Table A.

There is also provided a process for the preparation of compounds of formula (I):

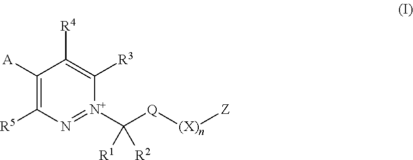

Wherein Q, Z, X, n, R¹, R², R³, R⁴, R⁵ and A are as defined herein;

comprising
(i) either
(a) reacting a compound of formula (H)

A-Hal formula (H)
wherein
A is as defined herein and Hal is a halogen or pseudo halogen, with a compound of formula (J)

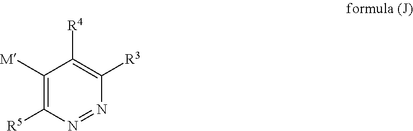

wherein
R³, R⁴ and R⁵ are as defined herein and M′ is an organostannane or an organoborane (e.g organoboronic acid, organoboronic ester or organotrifluoroborate), in the presence of a palladium catalyst, to give a compound of formula (X)

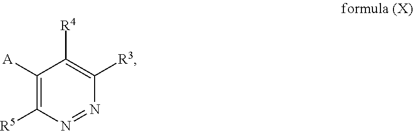

or
(b) reacting a compound of formula (K)

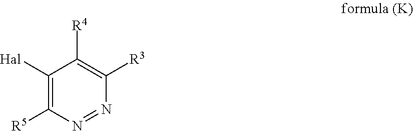

wherein R³, R⁴ and R⁵ are as defined herein and Hal is a halogen or pseudo halogen, with a compound of formula (L)

A-M′ formula (L)
wherein
A is as defined herein and M′ is an organostannane or an organoborane (e.g organoboronic acid, organoboronic ester or organotrifluoroborate), in the presence of a palladium catalyst, to give a compound of formula (X);
   (ii) reacting a compound of formula (X) with an alkylating agent of formula (W)

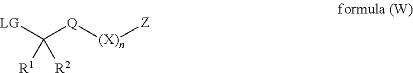

wherein R¹, R², Q, X, Z and n are as defined herein, and LG is a suitable leaving group (for example, halide or pseudohalide such as triflate, mesylate or tosylate), in an inert solvent or mixture of inert solvents, at a temperature of from −78° C. to 150° C., to give a compound of formula (I);
   (iii) optionally,
partially or fully hydrolysing a compound of formula (I) in the presence of a suitable acid.

According to the invention there is also provided the use of a compound of formula (J) as defined herein, in a process for the manufacture of a compound of formula (I) as defined herein. Preferably in a compound of formula (J) M′ is tributylstannane.

In another embodiment of the invention there is also provided the use of a compound of formula (X) as defined herein, in a process for the manufacture of a compound of formula (I) as defined herein. Preferably, the compound of formula (X) is selected from the group consisting of 4-(2-methyltetrazol-5-yl)pyridazine, 4-(4-methyl-1,2,4-triazol-3-yl)pyridazine, 4-(1-methyl-1,2,4-triazol-3-yl)pyridazine, 4-(2-methyl-1,2,4-triazol-3-yl)pyridazine, 3-pyridazin-4-ylisoxazole, 2-pyridazin-4-yloxazole, 5-methyl-2-pyridazin-4-yl-oxazole, 4-methyl-2-pyridazin-4-yl-oxazole, 2-pyridazin-4-ylthiazole, 4-methyl-2-pyridazin-4-yl-thiazole, 5-pyridazin-4-yl-1,3,4-thiadiazol-2-amine, 2-methyl-5-pyridazin-4-yl-1,3,4-thiadiazole, 4-(3-methyltriazol-4-yl)pyridazine, 5-pyridazin-4-yl-1,2,4-thiadiazole, 5-pyridazin-4-ylisothiazole, 3-methyl-5-pyridazin-4-yl-isothiazole, 5-pyridazin-4-ylisoxazole, 3-pyridazin-4-yl-1,2,4-thiadiazole and 3-pyridazin-4-ylisothiazole.

According to the invention there is also provided the novel intermediates of formula (X), wherein a compound of formula (X) selected from the group consisting of 4-(2-methyltetrazol-5-yl)pyridazine, 4-(1-methyl-1,2,4-triazol-3-yl)pyridazine, 4-(2-methyl-1,2,4-triazol-3-yl)pyridazine, 3-pyridazin-4-ylisoxazole, 5-methyl-2-pyridazin-4-yl-oxazole, 4-methyl-2-pyridazin-4-yl-oxazole, 2-methyl-5-pyridazin-4-yl-1,3,4-thiadiazole, 4-(3-methyltriazol-4-yl)pyridazine, 5-pyridazin-4-yl-1,2,4-thiadiazole, 5-pyridazin-4-ylisothiazole, 3-methyl-5-pyridazin-4-yl-isothiazole, 5-pyridazin-4-ylisoxazole, 3-pyridazin-4-yl-1,2,4-thiadiazole and 3-pyridazin-4-ylisothiazole.

It should be understood that compounds of formula (I) may exist/be manufactured in ‘procidal form’, wherein they comprise a group ‘G’. Such compounds are referred to herein as compounds of formula (I-IV).

G is a group which may be removed in a plant by any appropriate mechanism including, but not limited to, metabolism and chemical degradation to give a compound of formula (I-I) (I-I) or (I-II), wherein Z contains an acidic proton, for example see the scheme below:

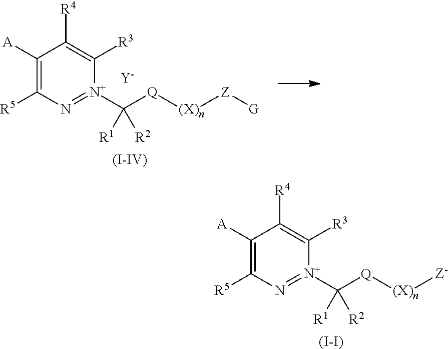

Whilst such G groups may be considered as ‘procidal’, and thus yield active herbicidal compounds once removed, compounds comprising such groups may also exhibit herbicidal activity in their own right. In such cases in a compound of formula (I-IV), Z-G may include but is not limited to, any one of (G1) to (G7) below and E indicates the point of attachment to the remaining part of a compound of formula (I):

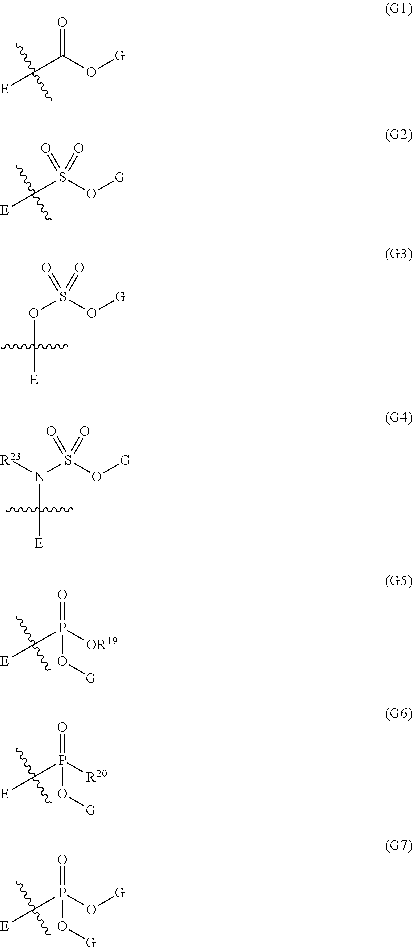

In embodiments where Z-G is (G1) to (G7), G, R¹⁹, R²⁰, R²¹, R²² and R²¹ are defined as follows:

G is C₁-C₆alkyl, C₂-C₆alkenyl, C₂-C₆alkynyl, —C(R²¹R²²)OC(O)R¹⁹, phenyl or phenyl-C₁-C₄alkyl-, wherein said phenyl moiety is optionally substituted by 1 to 5substituents independently selected from halo, cyano, nitro, C₁-C₆alkyl, C₁-C₆haloalkyl or C₁-C₆alkoxy.

R¹⁹ is C₁-C₆alkyl or phenyl,

R²⁰ is hydroxy, C₁-C₆alkyl, C₁-C₆alkoxy or phenyl,

R²¹ is hydrogen or methyl,

R²² is hydrogen or methyl,

R²³ is hydrogen or C₁-C₆alkyl.

In one embodiment there is provided a compound of formula (I) or an agronomically acceptable salt or zwitterionic species thereof:

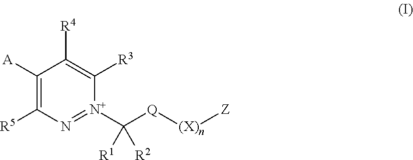

wherein
R¹ is selected from the group consisting of hydrogen, halogen, C₁-C₆alkyl, C₁-C₆fluoroalkyl, —OR⁷, —NHS(O)₂R¹⁵, —NHC(O)R¹⁵, —NHC(O)OR¹⁵, —NHC(O)NR¹⁶R¹⁷, —N(R⁷)₂ and —S(O)ᵣR¹⁵;
R² is selected from the group consisting of hydrogen, halogen, C₁-C₃alkyl and C₁-C₆fluoroalkyl;
and wherein when R¹ is selected from the group consisting of —OR, —NHS(O)₂R¹⁵, —NHC(O)R¹⁵, —NHC(O)OR¹⁵, —NHC(O)NR¹⁶R¹⁷, —N(R⁷)₂ and —S(O)ᵣR¹⁵, R² is selected from the group consisting of hydrogen and C₁-C₆alkyl; or
R¹ and R² together with the carbon atom to which they are attached form a C₃-C₆cycloalkyl ring;
Q is (CR^{1a}R^{2b})ₘ;
m is 0, 1, 2 or 3;
each R^{1a} and R^{2b} are independently selected from the group consisting of hydrogen, halogen, C₁-C₃alkyl, C₁-C₆fluoroalkyl, —OH, —OR⁷, —NH₂, —NHR⁷, —N(R⁷)₂ and —S(O)ᵣR¹⁵; or
each R^{1a} and R^{2b} together with the carbon atom to which they are attached form a C₃-C₆cycloalkyl ring;
R³, R⁴ and R⁵ are independently selected from the group consisting of hydrogen, halogen, cyano, nitro, —S(O)ᵣR¹⁵, C₁-C₆alkyl, C₁-C₆fluoroalkyl, C₁-C₆fluoroalkoxy, C₁-C₆alkoxy, C₃-C₆cycloalkyl and —N(R⁶)₂;

each R⁶ is independently selected from hydrogen and C₁-C₃alkyl;

each R⁷ is independently selected from the group consisting of C₁-C₆alkyl, —S(O)₂R¹⁵, —C(O)R¹⁵, —C(O)OR¹⁵ and —C(O)NR¹⁶R¹⁷;
A is a 5-membered heteroaryl attached to the rest of the molecule via a ring carbon atom, which comprises 1, 2, 3 or 4 heteroatoms independently selected from the group consisting of N, O and S, and wherein the heteroaryl may, where feasible, be optionally substituted by 1, 2 or 3 R⁸ substituents, which may be the same or different,

and wherein when A is substituted on one or more ring carbon atoms, each R⁸ is independently selected from the group consisting of halogen, nitro, cyano, —NH₂, —NHR⁷, —N(R⁷)₂, —OH, —OR⁷, —S(O)ᵣR¹⁵, —NR⁶S(O)₂R¹⁵, —C(O)OR¹⁰, —C(O)R¹⁵, —C(O)NR¹⁶R¹⁷, —S(O)₂NR¹⁶R¹⁷, C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₃alkoxyC₁-C₃alkoxy-, C₁-C₆haloalkoxy, C₁-C₃haloalkoxyC₁-C₃alkyl-, C₃-C₆alkenyloxy, C₃-C₆alkynyloxy, —C(R⁶)═NOR⁶, phenyl and a 5- or 6-membered heteroaryl, which comprises 1, 2, 3 or 4 heteroatoms independently selected from N, O and S, and wherein said phenyl or heteroaryl are optionally substituted by 1, 2 or 3 R⁹ substituents, which may be the same or different;

and/or
when A is substituted on a ring nitrogen atom, R⁸ is selected from the group consisting of —OR⁷, C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₆alkoxyC₁-C₃alkoxy-, C₁-C₆haloalkoxy, C₁-C₃haloalkoxyC₁-C₃alkyl-, C₃-C₆alkenyloxy and C₃-C₆alkynyloxy; and
each R⁹ is independently selected from the group consisting of halogen, cyano, —N(R⁶)₂, C₁-C₄alkyl, C₁-C₄alkoxy, C₁-C₄haloalkyl and C₁-C₄haloalkoxy;
X is independently selected from the group consisting of C₃-C₆cycloalkyl, phenyl, a 5- or 6-membered heteroaryl, which comprises 1, 2, 3 or 4 heteroatoms independently selected from N, O and S, and a 4- to 6-membered heterocyclyl, which comprises 1, 2 or 3 heteroatoms independently selected from N, O and S, and wherein said cycloalkyl, phenyl, heteroaryl or heterocyclyl moieties are optionally substituted by 1 or 2 substituents, which may be the same or different, selected from R⁹, and wherein the aforementioned CR¹R² and Z, or Q and Z, moieties may be attached at any position of said cycloalkyl, phenyl, heteroaryl or heterocyclyl moieties;
n is 0 or 1;
Z is selected from the group consisting of —C(O)OR¹⁰, —CH₂H, —CHO, —C(O)NHOR¹¹, —C(O)NHCN, —OC(O)NHOR¹¹, —OC(O)NHCN, —NR⁶C(O)NHOR¹¹, —NR⁶C(O)NHCN, —C(O)NHS(O)₂R¹², —OC(O)NHS(O)₂R¹², —NR⁶C(O)NHS(O)₂R¹², —S(O)₂OR¹⁰, —OS(O)₂OR¹⁰, —NR⁶S(O)₂OR¹⁰, —NRS(O)OR¹⁰, —NHS(O)₂R¹⁴, —S(O)OR¹⁰, —OS(O)OR¹⁰, —S(O)₂NHCN, —S(O)₂NHC(O)R¹⁸, —S(O)₂NHS(O)₂R¹², —OS(O)₂NHCN, —OS(O)₂NHS(O)₂R¹², —OS(O)₂NHC(O)R¹⁸, —NR⁶S(O)₂NHCN, —NR⁶S(O)₂NHC(O)R¹⁸, —N(OH)C(O)R¹⁵, —ONHC(O)R¹⁵, —NR⁶S(O)₂NHS(O)₂R¹², —P(O)(R¹³)(OR¹⁰), —P(O)H(OR¹⁰), —OP(O)(R¹³)(OR¹⁰), —NR⁶P(O)(R¹³)(OR¹⁰) and tetrazole;
R¹⁰ is selected from the group consisting of hydrogen, C₁-C₆alkyl, phenyl and benzyl, and wherein said phenyl or benzyl are optionally substituted by 1, 2 or 3 R⁹ substituents, which may be the same or different;
R¹¹ is selected from the group consisting of hydrogen, C₁-C₆alkyl and phenyl, and wherein said phenyl is optionally substituted by 1, 2 or 3 R⁹ substituents, which may be the same or different;
R¹² is selected from the group consisting of C₁-C₆alkyl, C₁-C₆haloalkyl, C₁-C₆alkoxy, —OH, —N(R⁶)₂ and phenyl, and wherein said phenyl is optionally substituted by 1, 2 or 3 R⁹ substituents, which may be the same or different;
R¹³ is selected from the group consisting of —OH, C₁-C₆alkyl, C₁-C₆alkoxy and phenyl;
R¹⁴ is C₁-C₆haloalkyl;
R¹⁵ is selected from the group consisting of C₁-C₆alkyl and phenyl, and wherein said phenyl is optionally substituted by 1, 2 or 3 R⁹ substituents, which may be the same or different;
R¹⁶ and R¹⁷ are independently selected from the group consisting of hydrogen and C₁-C₆alkyl; or
R¹⁶ and R¹⁷ together with the nitrogen atom to which they are attached form a 4- to 6-membered heterocyclyl ring which optionally comprises one additional heteroatom independently selected from N, O and S;
R¹⁸ is selected from the group consisting of hydrogen, C₁-C₆alkyl, C₁-C₆haloalkyl, C₁-C₆alkoxy, —N(R⁶)₂ and phenyl, and wherein said phenyl is optionally substituted by 1, 2 or 3 R⁹ substituents, which may be the same or different;
and

r is 0, 1 or 2.

The compounds in Tables 1 to 57 below illustrate the compounds of the invention. The skilled person would understand that the compounds of formula (I) may exist as an agronomically acceptable salt, a zwitterion or an agronomically acceptable salt of a zwitterion as described hereinbefore.

TABLE 1
This table discloses 53 specific compounds of the formula (T-1):
(T-1)

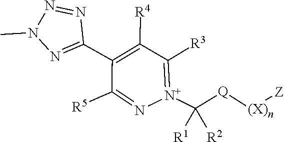

| Compound | | | | | | |
|---|---|---|---|---|---|---|
| number | R³ | R⁴ | R⁵ | Z | m | Q |
| 1.001 | H | H | H | —C(O)OH | 0 | — |
| 1.002 | H | H | H | —C(O)OMe | 0 | — |
| 1.003 | H | H | H | —C(O)NHOMe | 0 | — |
| 1.004 | H | H | H | —OC(O)NHOMe | 0 | — |
| 1.005 | H | H | H | —NHC(O)NHOMe | 0 | — |
| 1.006 | H | H | H | —NMeC(O)NHOMe | 0 | — |
| 1.007 | H | H | H | —C(O)NHS(O)₂Me | 0 | — |
| 1.008 | H | H | H | —OC(O)NHS(O)₂Me | 0 | — |
| 1.009 | H | H | H | —NHC(O)NHS(O)₂Me | 0 | — |
| 1.010 | H | H | H | —NMeC(O)NHS(O)₂Me | 0 | — |
| 1.011 | H | H | H | —S(O)₂OH | 0 | — |
| 1.012 | H | H | H | —OS(O)₂OH | 0 | — |
| 1.013 | H | H | H | —NHS(O)₂OH | 0 | — |
| 1.014 | H | H | H | —NMeS(O)₂OH | 0 | — |
| 1.015 | H | H | H | —S(O)OH | 0 | — |
| 1.016 | H | H | H | —OS(O)OH | 0 | — |
| 1.017 | H | H | H | —NHS(O)OH | 0 | — |
| 1.018 | H | H | H | —NMeS(O)OH | 0 | — |
| 1.019 | H | H | H | —NHS(O)₂CF₃ | 0 | — |
| 1.020 | H | H | H | —S(O)₂NHC(O)Me | 0 | — |
| 1.021 | H | H | H | —OS(O)₂NHC(O)Me | 0 | — |
| 1.022 | H | H | H | —NHS(O)₂NHC(O)Me | 0 | — |
| 1.023 | H | H | H | —NMeS(O)₂NHC(O)Me | 0 | — |
| 1.024 | H | H | H | —P(O)(OH)(OMe) | 0 | — |
| 1.025 | H | H | H | —P(O)(OH)(OH) | 0 | — |
| 1.026 | H | H | H | —OP(O)(OH)(OMe) | 0 | — |
| 1.027 | H | H | H | —OP(O)(OH)(OH) | 0 | — |
| 1.028 | H | H | H | —NHP(O)(OH)(OMe) | 0 | — |
| 1.029 | H | H | H | —NHP(O)(OH)(OH) | 0 | — |
| 1.030 | H | H | H | —NMeP(O)(OH)(OMe) | 0 | — |
| 1.031 | H | H | H | —NMeP(O)(OH)(OH) | 0 | — |
| 1.032 | H | H | H | —tetrazole | 0 | — |
| 1.033 | H | H | H | —S(O)₂OH | 1 | CH(NH₂) |
| 1.034 | H | H | H | —C(O)OH | 1 | CH(NH₂) |
| 1.035 | H | H | H | —S(O)₂OH | 2 | CH(OH)CH₂ |
| 1.036 | H | H | H | —C(O)OH | 2 | CH(OH)CH₂ |
| 1.037 | H | H | H | —S(O)₂OH | 1 | CH(OH) |
| 1.038 | H | H | H | —C(O)OH | 1 | CH(OH) |
| 1.039 | H | H | H | —C(O)NHCN | 0 | — |
| 1.040 | H | H | H | —OC(O)NHCN | 0 | — |
| 1.041 | H | H | H | —NHC(O)NHCN | 0 | — |
| 1.042 | H | H | H | —NMeC(O)NHCN | 0 | — |
| 1.043 | H | H | H | —S(O)₂NHCN | 0 | — |
| 1.044 | H | H | H | —OS(O)₂NHCN | 0 | — |
| 1.045 | H | H | H | —NHS(O)₂NHCN | 0 | — |
| 1.046 | H | H | H | —NMeS(O)₂NHCN | 0 | — |
| 1.047 | H | H | H | —S(O)₂NHS(O)₂Me | 0 | — |
| 1.048 | H | H | H | —OS(O)₂NHS(O)₂Me | 0 | — |
| 1.049 | H | H | H | —NHS(O)₂NHS(O)₂Me | 0 | — |
| 1.050 | H | H | H | —NMeS(O)₂NHS(O)₂Me | 0 | — |
| 1.051 | H | H | H | —P(O)H(OH) | 0 | — |
| 1.052 | H | H | H | —N(OH)C(O)Me | 0 | — |
| 1.053 | H | H | H | —ONHC(O)Me | 0 | — |

wherein m, Q, R³, R⁴, R⁵ and Z are as defined in Table 1, R¹ and R² are hydrogen and n is 0.

TABLE 2
This table discloses 49 specific compounds of the formula (T-2):
(T-2)

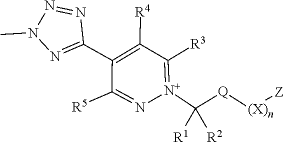

| Compound | | | | | | |
|---|---|---|---|---|---|---|
| number | R³ | R⁴ | R⁵ | Z | m | Q |
| 2.001 | H | H | H | —C(O)OH | 1 | CH₂ |
| 2.002 | H | H | H | —C(O)OMe | 1 | CH₂ |
| 2.003 | H | H | H | —C(O)NHOMe | 1 | CH₂ |
| 2.004 | H | H | H | —OC(O)NHOMe | 1 | CH₂ |
| 2.005 | H | H | H | —NHC(O)NHOMe | 1 | CH₂ |
| 2.006 | H | H | H | —NMeC(O)NHOMe | 1 | CH₂ |
| 2.007 | H | H | H | —C(O)NHS(O)₂Me | 1 | CH₂ |
| 2.008 | H | H | H | —OC(O)NHS(O)₂Me | 1 | CH₂ |
| 2.009 | H | H | H | —NHC(O)NHS(O)₂Me | 1 | CH₂ |
| 2.010 | H | H | H | —NMeC(O)NHS(O)₂Me | 1 | CH₂ |
| 2.011 | H | H | H | —S(O)₂OH | 1 | CH₂ |
| 2.012 | H | H | H | —OS(O)₂OH | 1 | CH₂ |
| 2.013 | H | H | H | —NHS(O)₂OH | 1 | CH₂ |
| 2.014 | H | H | H | —NMeS(O)₂OH | 1 | CH₂ |
| 2.015 | H | H | H | —S(O)OH | 1 | CH₂ |
| 2.016 | H | H | H | —OS(O)OH | 1 | CH₂ |
| 2.017 | H | H | H | —NHS(O)OH | 1 | CH₂ |
| 2.018 | H | H | H | —NMeS(O)OH | 1 | CH₂ |
| 2.019 | H | H | H | —NHS(O)₂CF₃ | 1 | CH₂ |
| 2.020 | H | H | H | —S(O)₂NHC(O)Me | 1 | CH₂ |
| 2.021 | H | H | H | —OS(O)₂NHC(O)Me | 1 | CH₂ |
| 2.022 | H | H | H | —NHS(O)₂NHC(O)Me | 1 | CH₂ |
| 2.023 | H | H | H | —NMeS(O)₂NHC(O)Me | 1 | CH₂ |
| 2.024 | H | H | H | —P(O)(OH)(OMe) | 1 | CH₂ |
| 2.025 | H | H | H | —P(O)(OH)(OH) | 1 | CH₂ |
| 2.026 | H | H | H | —OP(O)(OH)(OMe) | 1 | CH₂ |
| 2.027 | H | H | H | —OP(O)(OH)(OH) | 1 | CH₂ |
| 2.028 | H | H | H | —NHP(O)(OH)(OMe) | 1 | CH₂ |
| 2.029 | H | H | H | —NHP(O)(OH)(OH) | 1 | CH₂ |
| 2.030 | H | H | H | —NMeP(O)(OH)(OMe) | 1 | CH₂ |
| 2.031 | H | H | H | —NMeP(O)(OH)(OH) | 1 | CH₂ |
| 2.032 | H | H | H | —tetrazole | 1 | CH₂ |
| 2.033 | H | H | H | —S(O)₂OH | 2 | CH₂CH(NH₂) |
| 2.034 | H | H | H | —C(O)OH | 2 | CH₂CH(NH₂) |
| 2.035 | H | H | H | —C(O)NHCN | 1 | CH₂ |
| 2.036 | H | H | H | —OC(O)NHCN | 1 | CH₂ |
| 2.037 | H | H | H | —NHC(O)NHCN | 1 | CH₂ |
| 2.038 | H | H | H | —NMeC(O)NHCN | 1 | CH₂ |
| 2.039 | H | H | H | —S(O)₂NHCN | 1 | CH₂ |
| 2.040 | H | H | H | —OS(O)₂NHCN | 1 | CH₂ |
| 2.041 | H | H | H | —NHS(O)₂NHCN | 1 | CH₂ |
| 2.042 | H | H | H | —NMeS(O)₂NHCN | 1 | CH₂ |
| 2.043 | H | H | H | —S(O)₂NHS(O)₂Me | 1 | CH₂ |
| 2.044 | H | H | H | —OS(O)₂NHS(O)₂Me | 1 | CH₂ |
| 2.045 | H | H | H | —NHS(O)₂NHS(O)₂Me | 1 | CH₂ |
| 2.046 | H | H | H | —NMeS(O)₂NHS(O)₂Me | 1 | CH₂ |
| 2.047 | H | H | H | —P(O)H(OH) | 1 | CH₂ |
| 2.048 | H | H | H | —N(OH)C(O)Me | 1 | CH₂ |
| 2.049 | H | H | H | —ONHC(O)Me | 1 | CH₂ |

wherein m, Q, R³, R⁴, R⁵ and Z are as defined in Table 2, R¹ and R² are hydrogen and n is 0.

TABLE 3
This table discloses 49 specific compounds of the formula (T-3):
(T-3)

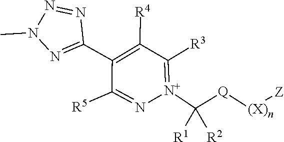

| Compound | | | | | | |
|---|---|---|---|---|---|---|
| number | R³ | R⁴ | R⁵ | Z | m | Q |
| 3.001 | H | H | H | —C(O)OH | 2 | CH₂CH₂ |
| 3.002 | H | H | H | —C(O)OMe | 2 | CH₂CH₂ |
| 3.003 | H | H | H | —C(O)NHOMe | 2 | CH₂CH₂ |
| 3.004 | H | H | H | —OC(O)NHOMe | 2 | CH₂CH₂ |
| 3.005 | H | H | H | —NHC(O)NHOMe | 2 | CH₂CH₂ |
| 3.006 | H | H | H | —NMeC(O)NHOMe | 2 | CH₂CH₂ |
| 3.007 | H | H | H | —C(O)NHS(O)₂Me | 2 | CH₂CH₂ |
| 3.008 | H | H | H | —OC(O)NHS(O)₂Me | 2 | CH₂CH₂ |
| 3.009 | H | H | H | —NHC(O)NHS(O)₂Me | 2 | CH₂CH₂ |
| 3.010 | H | H | H | —NMeC(O)NHS(O)₂Me | 2 | CH₂CH₂ |
| 3.011 | H | H | H | —S(O)₂OH | 2 | CH₂CH₂ |
| 3.012 | H | H | H | —OS(O)₂OH | 2 | CH₂CH₂ |
| 3.013 | H | H | H | —NHS(O)₂OH | 2 | CH₂CH₂ |
| 3.014 | H | H | H | —NMeS(O)₂OH | 2 | CH₂CH₂ |
| 3.015 | H | H | H | —S(O)OH | 2 | CH₂CH₂ |
| 3.016 | H | H | H | —OS(O)OH | 2 | CH₂CH₂ |
| 3.017 | H | H | H | —NHS(O)OH | 2 | CH₂CH₂ |
| 3.018 | H | H | H | —NMeS(O)OH | 2 | CH₂CH₂ |
| 3.019 | H | H | H | —NHS(O)₂CF₃ | 2 | CH₂CH₂ |
| 3.020 | H | H | H | —S(O)₂NHC(O)Me | 2 | CH₂CH₂ |
| 3.021 | H | H | H | —OS(O)₂NHC(O)Me | 2 | CH₂CH₂ |
| 3.022 | H | H | H | —NHS(O)₂NHC(O)Me | 2 | CH₂CH₂ |
| 3.023 | H | H | H | —NMeS(O)₂NHC(O)Me | 2 | CH₂CH₂ |
| 3.024 | H | H | H | —P(O)(OH)(OMe) | 2 | CH₂CH₂ |
| 3.025 | H | H | H | —P(O)(OH)(OH) | 2 | CH₂CH₂ |
| 3.026 | H | H | H | —OP(O)(OH)(OMe) | 2 | CH₂CH₂ |
| 3.027 | H | H | H | —OP(O)(OH)(OH) | 2 | CH₂CH₂ |
| 3.028 | H | H | H | —NHP(O)(OH)(OMe) | 2 | CH₂CH₂ |
| 3.029 | H | H | H | —NHP(O)(OH)(OH) | 2 | CH₂CH₂ |
| 3.030 | H | H | H | —NMeP(O)(OH)(OMe) | 2 | CH₂CH₂ |
| 3.031 | H | H | H | —NMeP(O)(OH)(OH) | 2 | CH₂CH₂ |
| 3.032 | H | H | H | —tetrazole | 2 | CH₂CH₂ |
| 3.033 | H | H | H | —S(O)₂OH | 3 | CH₂CH₂CH(NH₂) |
| 3.034 | H | H | H | —C(O)OH | 3 | CH₂CH₂CH(NH₂) |
| 3.035 | H | H | H | —C(O)NHCN | 2 | CH₂CH₂ |
| 3.036 | H | H | H | —OC(O)NHCN | 2 | CH₂CH₂ |
| 3.037 | H | H | H | —NHC(O)NHCN | 2 | CH₂CH₂ |
| 3.038 | H | H | H | —NMeC(O)NHCN | 2 | CH₂CH₂ |
| 3.039 | H | H | H | —S(O)₂NHCN | 2 | CH₂CH₂ |
| 3.040 | H | H | H | —OS(O)₂NHCN | 2 | CH₂CH₂ |
| 3.041 | H | H | H | —NHS(O)₂NHCN | 2 | CH₂CH₂ |
| 3.042 | H | H | H | —NMeS(O)₂NHCN | 2 | CH₂CH₂ |
| 3.043 | H | H | H | —S(O)₂NHS(O)₂Me | 2 | CH₂CH₂ |
| 3.044 | H | H | H | —OS(O)₂NHS(O)₂Me | 2 | CH₂CH₂ |
| 3.045 | H | H | H | —NHS(O)₂NHS(O)₂Me | 2 | CH₂CH₂ |
| 3.046 | H | H | H | —NMeS(O)₂NHS(O)₂Me | 2 | CH₂CH₂ |
| 3.047 | H | H | H | —P(O)H(OH) | 2 | CH₂CH₂ |
| 3.048 | H | H | H | —N(OH)C(O)Me | 2 | CH₂CH₂ |
| 3.049 | H | H | H | —ONHC(O)Me | 2 | CH₂CH₂ |

wherein m, Q, R³, R⁴, R⁵ and Z are as defined in Table 3, R¹ and R² are hydrogen and n is 0.

TABLE 4
This table discloses 53 specific compounds of the formula (T-4):

| | |
|---|---|
| 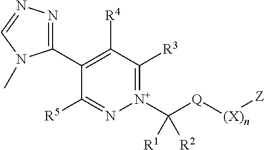 | (T-4) |

wherein m, Q, R³, R⁴, R⁵ and Z are as defined above in Table 1, R¹ and R² are hydrogen and n is 0.

TABLE 5
This table discloses 49 specific compounds of the formula (T-5):

| | |
|---|---|
| 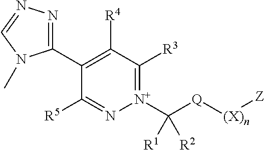 | (T-5) |

wherein m, Q, R³, R⁴, R⁵ and Z are as defined above in Table 2, R¹ and R² are hydrogen and n is 0.

TABLE 6
This table discloses 49 specific compounds of the formula (T-6):

| | |
|---|---|
| 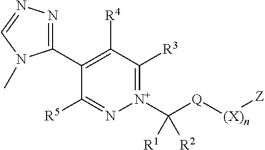 | (T-6) |

wherein m, Q, R³, R⁴, R⁵ and Z are as defined above in Table 3, R¹ and R² are hydrogen and n is 0.

TABLE 7
This table discloses 53 specific compounds of the formula (T-7):

| | |
|---|---|
| 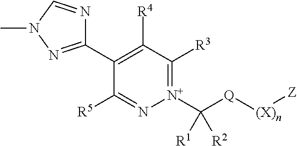 | (T-7) |

wherein m, Q, R³, R⁴, R⁵ and Z are as defined above in Table 1, R¹ and R² are hydrogen and n is 0.

TABLE 8
This table discloses 49 specific compounds of the formula (T-8):

| | |
|---|---|
| 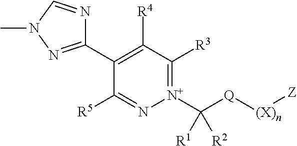 | (T-8) |

wherein m, Q, R³, R⁴, R⁵ and Z are as defined above in Table 2, R¹ and R² are hydrogen and n is 0.

TABLE 9
This table discloses 49 specific compounds of the formula (T-9):

| | |
|---|---|
| 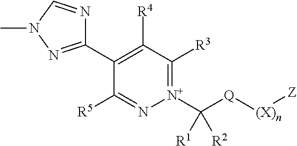 | (T-9) |

wherein m, Q, R³, R⁴, R⁵ and Z are as defined above in Table 3, R¹ and R² are hydrogen and n is 0.

TABLE 10
This table discloses 53 specific compounds of the formula (T-10):

| | |
|---|---|
| 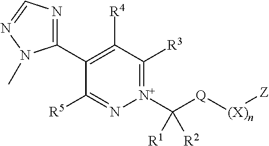 | (T-10) |

wherein m, Q, R³, R⁴, R⁵ and Z are as defined above in Table 1, R¹ and R² are hydrogen and n is 0.

TABLE 11
This table discloses 49 specific compounds of the formula (T-11):

| | |
|---|---|
| 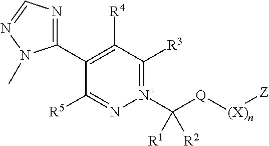 | (T-11) |

wherein m, Q, R³, R⁴, R⁵ and Z are as defined above in Table 2, R¹ and R² are hydrogen and n is 0.

TABLE 12
This table discloses 49 specific compounds of the formula (T-12):

| | |
|---|---|
| 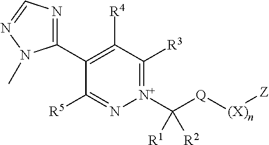 | (T-12) |

wherein m, Q, R³, R⁴, R⁵ and Z are as defined above in Table 3, R¹ and R² are hydrogen and n is 0.

TABLE 13
This table discloses 53 specific compounds of the formula (T-13):

| | |
|---|---|
| 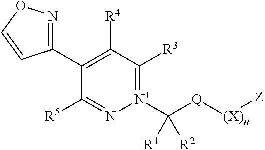 | (T-13) |

wherein m, Q, R³, R⁴, R⁵ and Z are as defined above in Table 1, R¹ and R² are hydrogen and n is 0.

TABLE 14
This table discloses 49 specific compounds of the formula (T-14):

| | |
|---|---|
| 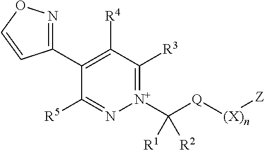 | (T-14) |

wherein m, Q, R³, R⁴, R⁵ and Z are as defined above in Table 2, R¹ and R² are hydrogen and n is 0.

TABLE 15
This table discloses 49 specific compounds of the formula (T-15):

| | |
|---|---|
| 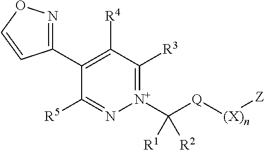 | (T-15) |

wherein m, Q, R³, R⁴, R⁵ and Z are as defined above in Table 3, R¹ and R² are hydrogen and n is 0.

TABLE 16
This table discloses 53 specific compounds of the formula (T-16):

| | |
|---|---|
| 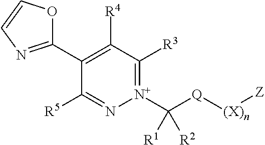 | (T-16) |

wherein m, Q, R³, R⁴, R⁵ and Z are as defined above in Table 1, R¹ and R² are hydrogen and n is 0.

TABLE 17
This table discloses 49 specific compounds of the formula (T-17):

| | |
|---|---|
| 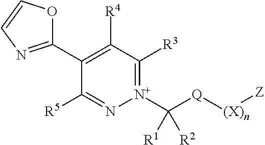 | (T-17) |

wherein m, Q, R³, R⁴, R⁵ and Z are as defined above in Table 2, R¹ and R² are hydrogen and n is 0.

TABLE 18
This table discloses 49 specific compounds of the formula (T-18):

| | |
|---|---|
| 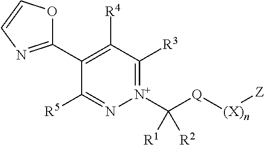 | (T-18) |

wherein m, Q, R³, R⁴, R⁵ and Z are as defined above in Table 3, R¹ and R² are hydrogen and n is 0.

TABLE 19
This table discloses 53 specific compounds of the formula (T-19):

| | |
|---|---|
| 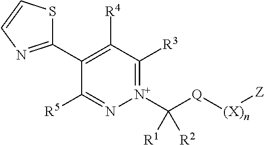 | (T-19) |

wherein m, Q, R³, R⁴, R⁵ and Z are as defined above in Table 1, R¹ and R² are hydrogen and n is 0.

TABLE 20
This table discloses 49 specific compounds of the formula (T-20):

| | |
|---|---|
| 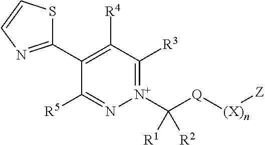 | (T-20) |

wherein m, Q, R³, R⁴, R⁵ and Z are as defined above in Table 2, R¹ and R² are hydrogen and n is 0.

TABLE 21
This table discloses 49 specific compounds of the formula (T-21):

| | |
|---|---|
| 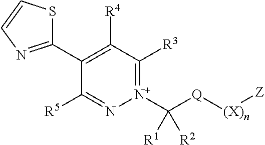 | (T-21) |

wherein m, Q, R³, R⁴, R⁵ and Z are as defined above in Table 3, R¹ and R² are hydrogen and n is 0.

TABLE 22
This table discloses 53 specific compounds of the formula (T-21):

| | |
|---|---|
| 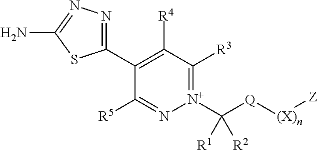 | (T-22) |

wherein m, Q, R³, R⁴, R⁵ and Z are as defined above in Table 1, R¹ and R² are hydrogen and n is 0.

TABLE 23
This table discloses 49 specific compounds of the formula (T-23):

| | |
|---|---|
| 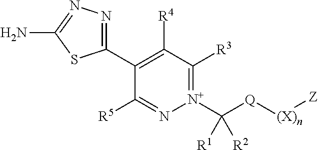 | (T-23) |

wherein m, Q, R³, R⁴, R⁵ and Z are as defined above in Table 2, R¹ and R² are hydrogen and n is 0.

TABLE 24
This table discloses 49 specific compounds of the formula (T-24):

| | |
|---|---|
| 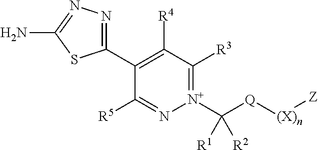 | (T-24) |

wherein m, Q, R³, R⁴, R⁵ and Z are as defined above in Table 3, R¹ and R² are hydrogen and n is 0.

TABLE 25
This table discloses 53 specific compounds of the formula (T-25):

| | |
|---|---|
| 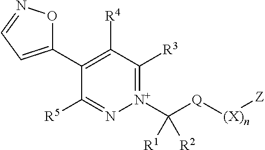 | (T-25) |

wherein m, Q, R³, R⁴, R⁵ and Z are as defined above in Table 1, R¹ and R² are hydrogen and n is 0.

TABLE 26
This table discloses 49 specific compounds of the formula (T-26):

| | |
|---|---|
| 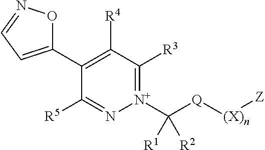 | (T-26) |

wherein m, Q, R³, R⁴, R⁵ and Z are as defined above in Table 2, R¹ and R² are hydrogen and n is 0.

TABLE 27
This table discloses 49 specific compounds of the formula (T-27):

| | |
|---|---|
| 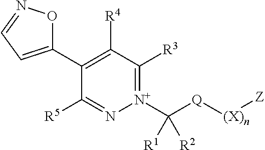 | (T-27) |

wherein m, Q, R³, R⁴, R⁵ and Z are as defined above in Table 3, R¹ and R² are hydrogen and n is 0.

TABLE 28
This table discloses 53 specific compounds of the formula (T-28):

| | |
|---|---|
| 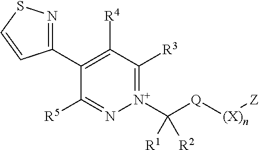 | (T-28) |

wherein m, Q, R³, R⁴, R⁵ and Z are as defined above in Table 1, R¹ and R² are hydrogen and n is 0.

TABLE 29
This table discloses 49 specific compounds of the formula (T-29):

| | |
|---|---|
| 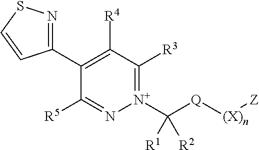 | (T-29) |

wherein m, Q, R³, R⁴, R⁵ and Z are as defined above in Table 2, R¹ and R² are hydrogen and n is 0.

TABLE 30
This table discloses 49 specific compounds of the formula (T-30):

| | |
|---|---|
| 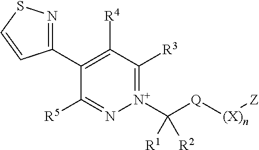 | (T-30) |

wherein m, Q, R³, R⁴, R⁵ and Z are as defined above in Table 3, R¹ and R² are hydrogen and n is 0.

TABLE 31
This table discloses 53 specific compounds of the formula (T-31):

| | |
|---|---|
| 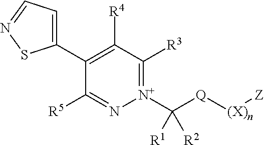 | (T-31) |

wherein m, Q, R³, R⁴, R⁵ and Z are as defined above in Table 1, R¹ and R² are hydrogen and n is 0.

TABLE 32
This table discloses 49 specific compounds of the formula (T-32):

| | |
|---|---|
| 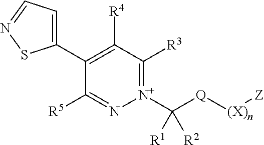 | (T-32) |

wherein m, Q, R³, R⁴, R⁵ and Z are as defined above in Table 2, R¹ and R² are hydrogen and n is 0.

TABLE 33
This table discloses 49 specific compounds of the formula (T-33):

| | |
|---|---|
| 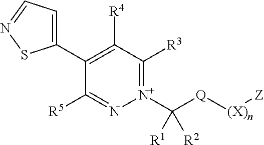 | (T-33) |

wherein m, Q, R³, R⁴, R⁵ and Z are as defined above in Table 3, R¹ and R² are hydrogen and n is 0.

TABLE 34
This table discloses 53 specific compounds of the formula (T-34):

| | |
|---|---|
| 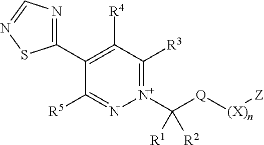 | (T-34) |

wherein m, Q, R³, R⁴, R⁵ and Z are as defined above in Table 1, R¹ and R² are hydrogen and n is 0.

TABLE 35
This table discloses 49 specific compounds of the formula (T-35):

| | |
|---|---|
| 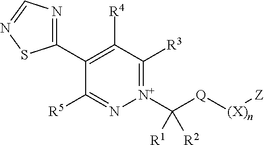 | (T-35) |

wherein m, Q, R³, R⁴, R⁵ and Z are as defined above in Table 2, R¹ and R² are hydrogen and n is 0.

TABLE 36
This table discloses 49 specific compounds of the formula (T-36):

| | |
|---|---|
| 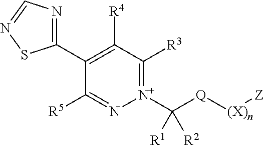 | (T-36) |

wherein m, Q, R³, R⁴, R⁵ and Z are as defined above in Table 3, R¹ and R² are hydrogen and n is 0.

TABLE 37
This table discloses 53 specific compounds of the formula (T-37):

| | |
|---|---|
| 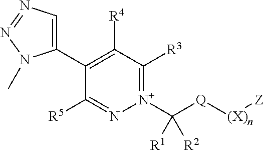 | (T-37) |

wherein m, Q, R³, R⁴, R⁵ and Z are as defined above in Table 1, R¹ and R² are hydrogen and n is 0.

TABLE 38
This table discloses 49 specific comounds of the formula (T-38):

| | |
|---|---|
| 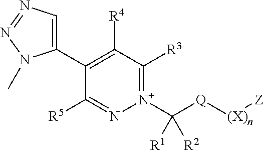 | (T-38) |

wherein m, Q, R³, R⁴, R⁵ and Z are as defined above in Table 2, R¹ and R² are hydrogen and n is 0.

TABLE 39
This table discloses 49 specific compounds of the formula (T-39):

| | |
|---|---|
| 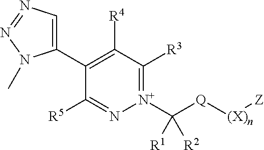 | (T-39) |

wherein m, Q, R³, R⁴, R⁵ and Z are as defined above in Table 3, R¹ and R² are hydrogen and n is 0.

TABLE 40
This table discloses 53 specific compounds of the formula (T-40):

| | |
|---|---|
| 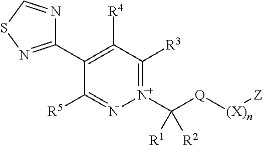 | (T-40) |

wherein m, Q, R³, R⁴, R⁵ and Z are as defined above in Table 1, R¹ and R² are hydrogen and n is 0.

TABLE 41
This table discloses 49 specific compounds of the formula (T-41):

| | |
|---|---|
| 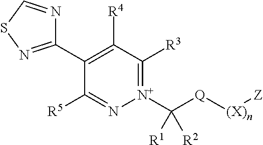 | (T-41) |

wherein m, Q, R³, R⁴, R⁵ and Z are as defined above in Table 2, R¹ and R² are hydrogen and n is 0.

TABLE 42
This table discloses 49 specific compounds of the formula (T-42):

| | |
|---|---|
| 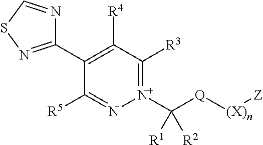 | (T-42) |

wherein m, Q, R³, R⁴, R⁵ and Z are as defined above in Table 3, R¹ and R² are hydrogen and n is 0.

TABLE 43
This table discloses 53 specific compounds of the formula (T-43):

| | |
|---|---|
| 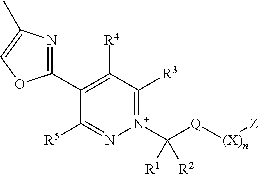 | (T-43) |

wherein m, Q, R³, R⁴, R⁵ and Z are as defined above in Table 1, R¹ and R² are hydrogen and n is 0.

TABLE 44
This table discloses 49 specific compounds of the formula (T-44):

| | |
|---|---|
| 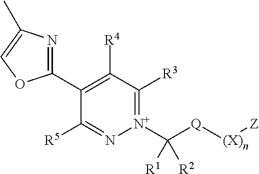 | (T-44) |

wherein m, Q, R³, R⁴, R⁵ and Z are as defined above in Table 2, R¹ and R² are hydrogen and n is 0.

TABLE 45
This table discloses 49 specific compounds of the formula (T-45):

| | |
|---|---|
| 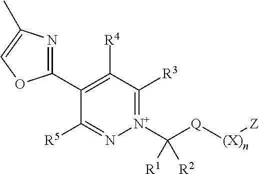 | (T-45) |

wherein m, Q, R³, R⁴, R⁵ and Z are as defined above in Table 3, R¹ and R² are hydrogen and n is 0.

TABLE 46
This table discloses 53 specific compounds of the formula (T-46):

| | |
|---|---|
| 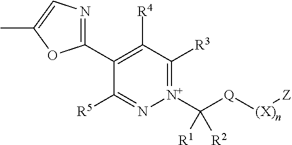 | (T-46) |

wherein m, Q, R³, R⁴, R⁵ and Z are as defined above in Table 1, R¹ and R² are hydrogen and n is 0.

TABLE 47
This table discloses 49 specific compounds of the formula (T-47):

| | |
|---|---|
| 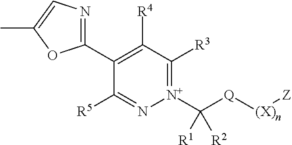 | (T-47) |

wherein m, Q, R³, R⁴, R⁵ and Z are as defined above in Table 2, R¹ and R² are hydrogen and n is 0.

TABLE 48
This table discloses 49 specific compounds of the formula (T-48):

| | |
|---|---|
| 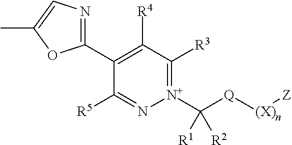 | (T-48) |

wherein m, Q, R³, R⁴, R⁵ and Z are as defined above in Table 3, R¹ and R² are hydrogen and n is 0.

TABLE 49
This table discloses 53 specific compounds of the formula (T-49):

| | |
|---|---|
| 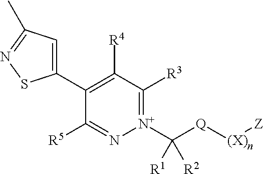 | (T-49) |

wherein m, Q, R³, R⁴, R⁵ and Z are as defined above in Table 1, R¹ and R² are hydrogen and n is 0.

TABLE 50
This table discloses 49 specific compounds of the formula (T-50):

| | |
|---|---|
| 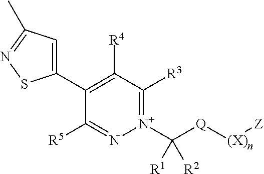 | (T-50) |

wherein m, Q, R³, R⁴, R⁵ and Z are as defined above in Table 2, R¹ and R² are hydrogen and n is 0.

TABLE 51
This table discloses 49 specific compounds of the formula (T-51):

| | |
|---|---|
| 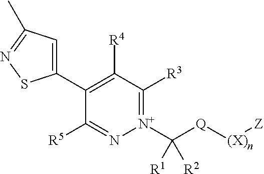 | (T-51) |

wherein m, Q, R³, R⁴, R⁵ and Z are as defined above in Table 3, R¹ and R² are hydrogen and n is 0.

TABLE 52
This table discloses 53 specific compounds of the formula (T-52):

| | |
|---|---|
| 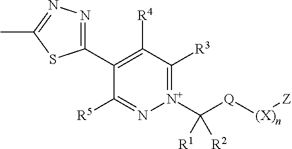 | (T-52) |

wherein m, Q, R³, R⁴, R⁵ and Z are as defined above in Table 1, R¹ and R² are hydrogen and n is 0.

TABLE 53
This table discloses 49 specific compounds of the formula (T-53):

| | |
|---|---|
| 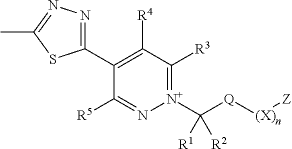 | (T-53) |

wherein m, Q, R³, R⁴, R⁵ and Z are as defined above in Table 2, R¹ and R² are hydrogen and n is 0.

TABLE 54
This table discloses 49 specific compounds of the formula (T-54):

| | |
|---|---|
| 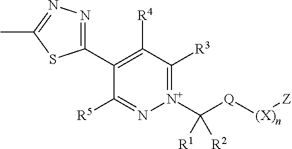 | (T-54) |

wherein m, Q, R³, R⁴, R⁵ and Z are as defined above in Table 3, R¹ and R² are hydrogen and n is 0.

TABLE 55
This table discloses 53 specific compounds of the formula (T-55):

| | |
|---|---|
| 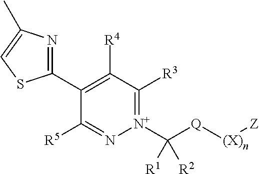 | (T-55) |

wherein m, Q, R³, R⁴, R⁵ and Z are as defined above in Table 1, R¹ and R² are hydrogen and n is 0.

TABLE 56
This table discloses 49 specific compounds of the formula (T-56):

| | |
|---|---|
| 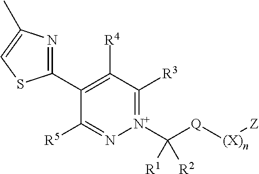 | (T-56) |

wherein m, Q, R³, R⁴, R⁵ and Z are as defined above in Table 2, R¹ and R² are hydrogen and n is 0.

TABLE 57
This table discloses 49 specific compounds of the formula (T-57):

| | |
|---|---|
| 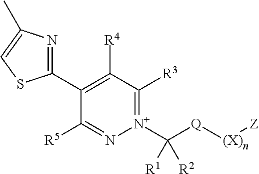 | (T-57) |

wherein m, Q, R³, R⁴, R⁵ and Z are as defined above in Table 3, R¹ and R² are hydrogen and n is 0.

The compounds of the present invention may be prepared according to the following schemes in which the substituents n, m, r, A, Q, X, Z, R¹, R², R^{1a}, R^{2b}, R³, R⁴, R⁵, R⁶, R⁷, R^{7a}, R^{7b}, R^{7c}, R⁸, R⁹, R¹⁰, R¹¹, R¹², R¹³, R¹⁴, R¹⁵, R^{15a}, R¹⁶, R¹⁷ and R¹⁸ are as defined hereinbefore unless explicitly stated otherwise. The compounds of the preceeding Tables 1 to 57 may thus be obtained in an analogous manner.

The compounds of formula (I) may be prepared by the alkylation of compounds of formula (X), wherein R³, R⁴, R⁵ and A are as defined for compounds of formula (I), with a suitable alkylating agent of formula (W), wherein R¹, R², Q, X, n and Z are as defined for compounds of formula (I) and LG is a suitable leaving group, for example, halide or pseudohalide such as triflate, mesylate or tosylate, in a suitable solvent at a suitable temperature, as described in reaction scheme 1. Example conditions include stirring a compound of formula (X) with an alkylating agent of formula (W) in a solvent, or mixture of solvents, such as acetone, dichloromethane, dichloroethane, N,N-dimethylformamide, acetonitrile, 1,4-dioxane, water, acetic acid or trifluroacetic acid at a temperature between −78° C. and 150° C. An alkylating agent of formula (W) may include, but is not limited to, bromoacetic acid, methyl bromoacetate, 3-bromopropionoic acid, methyl 3-bromopropionate, 2-bromo-N-methoxyacetamide, sodium 2-bromoethanesulphonate, 2,2-dimethylpropyl 2-(trifluoromethylsulfonyloxy)ethanesulfonate, 2-bromo-N-methanesulfonylacetamide, 3-bromo-N-methanesulfonylpropanamide, dimethoxyphosphorylmethyl trifluoromethanesulfonate, dimethyl 3-bromopropylphosphonate, 3-chloro-2,2-dimethyl-propanoic acid and diethyl 2-bromoethylphosphonate. Such alkylating agents and related compounds are either known in the literature or may be prepared by known literature methods. Compounds of formula (I) which may be described as esters of N-alkyl acids, which include, but are not limited to, esters of carboxylic acids, phosphonic acids, phosphinic acids, sulfonic acids and sulfinic acids, may be subsequently partially or fully hydrolysed by treatment with a suitable reagent, for example, aqueous hydrochloric acid or trimethylsilyl bromide, in a suitable solvent at a suitable temperature between 0° C. and 100° C.

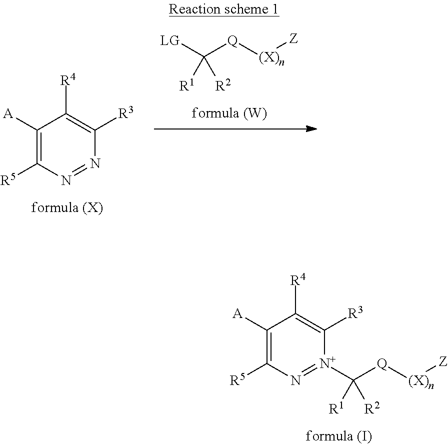

Additionally, compounds of formula (I) may be prepared by reacting compounds of formula (X), wherein R³, R⁴, R⁵ and A are as defined for compounds of formula (I), with a suitably activated electrophilic alkene of formula (B), wherein Z is —S(O)₂R¹⁰, —P(O)(R¹³)(OR¹⁰) or —C(O)OR¹⁰ and R¹, R², R^{1a}, R¹⁰ and R¹³ are as defined for compounds of formula (I), in a suitable solvent at a suitable temperature. Compounds of formula (B) are known in the literature, or may be prepared by known methods. Example reagents include, but are not limited to, acrylic acid, methacrylic acid, crotonic acid, 3,3-dimethylacrylic acid, methyl acrylate, ethene sulfonic acid, isopropyl ethylenesulfonate, 2,2-dimethylpropyl ethenesulfonate and dimethyl vinylphosphonate. The direct products of these reactions, which may be described as esters of N-alkyl acids, which include, but are not limited to, esters of carboxylic acids, phosphonic acids, phosphinic acids, sulfonic acids and sulfinic acids, may be subsequently partially or fully hydrolysed by treatment with a suitable reagent in a suitable solvent at a suitable temperature, as described in reaction scheme 2.

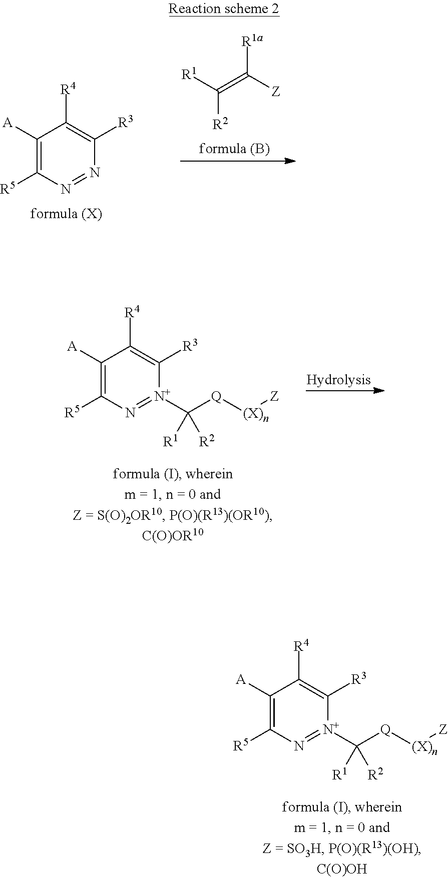

In a related reaction compounds of formula (I), wherein Q is C(R^{1a}R^{2b}), m is 1, 2 or 3, n=0 and Z is —S(O)₂OH, —OS(O)₂OH or —NR⁶S(O)₂H, may be prepared by the reaction of compounds of formula (X), wherein R³, R⁴, R⁵ and A are as defined for compounds of formula (I), with a cyclic alkylating agent of formula (E), (F) or (AF), wherein Y^{a} is C(R^{1a}R^{2b}), O or NR⁶ and R¹, R², R^{1a} and R^{2b} are as defined for compounds of formula (I), in a suitable solvent at a suitable temperature, as described in reaction scheme 3. Suitable solvents and suitable temperatures are as previously described. An alkylating agent of formula (E) or (F) may include, but is not limited to, 1,3-propanesultone, 1,4-butanesultone, ethylenesulfate, 1,3-propylene sulfate and 1,2,3-oxathiazolidine 2,2-dioxide. Such alkylating agents and related compounds are either known in the literature or may be prepared by known literature methods.

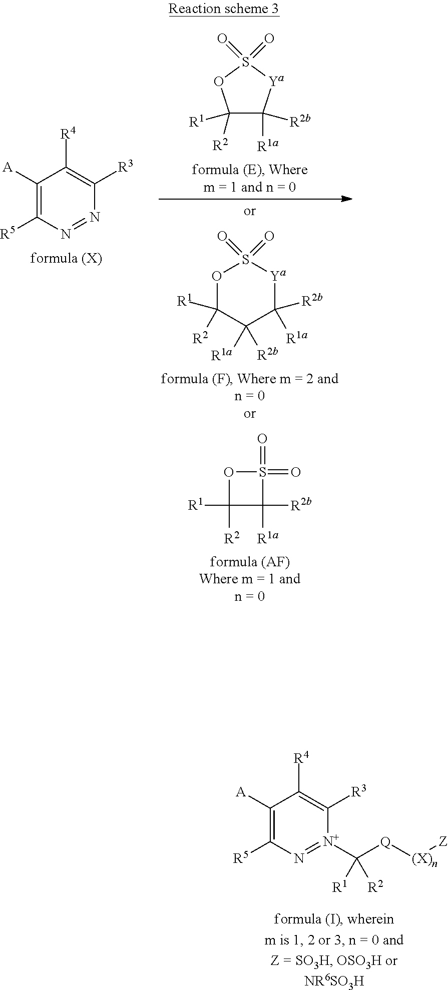

A compound of formula (I), wherein m is 0, n is 0 and Z is —S(O)₂OH, may be prepared from a compound of formula (I), wherein m is 0, n is 0 and Z is C(O)OR¹⁰, by treatment with trimethylsilylchlorosulfonate in a suitable solvent at a suitable temperature, as described in reaction scheme 4. Preferred conditions include heating the carboxylate precursor in neat trimethylsilylchlorosulfonate at a temperature between 25° C. and 150° C.

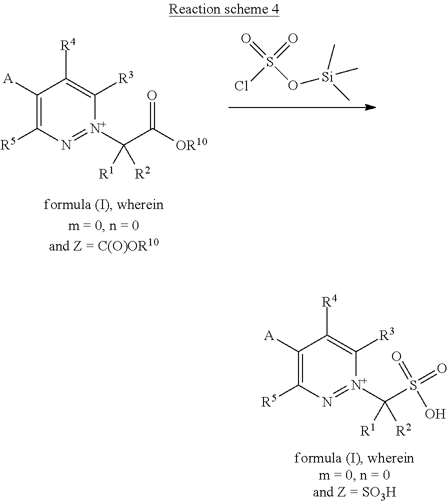

Furthermore, compounds of formula (I) may be prepared by reacting compounds of formula (X), wherein R³, R⁴, R⁵ and A are as defined for compounds of formula (I), with a suitable alcohol of formula (WW), wherein R¹, R², Q, X, n and Z are as defined for compounds of formula (I), under Mitsunobu-type conditions such as those reported by Petit et al, Tet. Lett. 2008, 49 (22), 3663. Suitable phosphines include triphenylphosphine, suitable azodicarboxylates include diisopropylazodicarboxylate and suitable acids include fluoroboric acid, triflic acid and bis(trifluoromethylsulfonyl)amine, as described in reaction scheme 5. Such alcohols are either known in the literature or may be prepared by known literature methods.

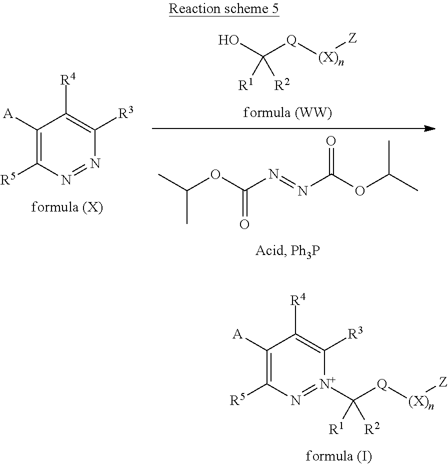

Compounds of formula (I) may also be prepared by reacting compounds of formula (C), wherein Q, Z, X, n, R¹, R², R³, R⁴, R⁵ and A are as defined for compounds of formula (I), with a hydrazine of formula (D) in a suitable solvent or mixture of solvents, in the presence of a suitable acid at a suitable temperature, between −78° C. and 150° C., as described in reaction scheme 6. Suitable solvents, or mixtures thereof, include, but are not limited to, alcohols, such as methanol, ethanol and isopropanol, water, aqueous hydrochloric acid, aqueous sulfuric acid, acetic acid and trifluoroacetic acid. Hydrazine compounds of formula (D), for example 2,2-dimethylpropyl 2-hydrazinoethanesulfonate, are either known in the literature or may be prepared by known literature procedures.

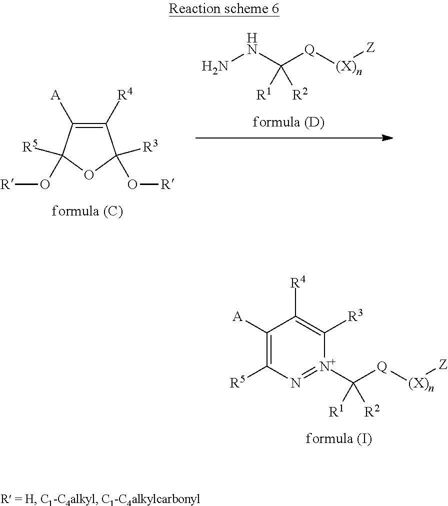

Compounds of formula (C) may be prepared by reacting compounds of formula (G), wherein R³, R⁴, R⁵ and A are as defined for compounds of formula (I), with an oxidising agent in a suitable solvent at a suitable temperature, between −78° C. and 150° C., optionally in the presence of a suitable base, as described in reaction scheme 7. Suitable oxidising agents include, but are not limited to, bromine and suitable solvents include, but are not limited to alcohols such as methanol, ethanol and isopropanol. Suitable bases include, but are not limited to, sodium bicarbonate, sodium carbonate, potassium bicarbonate, potassium carbonate and potassium acetate. Similar reactions are known in the literature (for example Hufford, D. L.; Tarbell, D. S.; Koszalka, T. R. J. Amer. Chem. Soc., 1952, 3014). Furans of formula (G) are known in the literature or may be prepared using literature methods. Example methods include, but are not limited to, transition metal cross-couplings such as Stille (for example Farina, V.; Krishnamurthy, V.; Scott, W. J. Organic Reactions, Vol. 50. 1997, and Gazzard, L. et al. J. Med. Chem., 2015, 5053), Suzuki-Miyaura (for example Ando, S.; Matsunaga, H.; Ishizuka, T. J. Org. Chem. 2017, 1266-1272, and Ernst, J. B.; Rakers, L.; Glorius, F. Synthesis, 2017, 260), Negishi (for example Yang, Y.; Oldenhius, N. J.; Buchwald, S. L. Angew. Chem. Int. Ed. 2013, 615, and Braendvang, M.; Gundersen, L. Bioorg. Med. Chem. 2005, 6360), and Kumada (for example Heravi, M. M.; Hajiabbasi, P. Monatsh. Chem., 2012, 1575). The coupling partners may be selected with reference to the specific cross-coupling reaction and target product. Transition metal catalysts, ligands, bases, solvents and temperatures may be selected with reference to the desired cross-coupling and are known in the literature. Cross-coupling reactions using pseudo halogens, including but not limited to, triflates, mesylates, tosylates and anisoles, may also be achieved under related conditions.

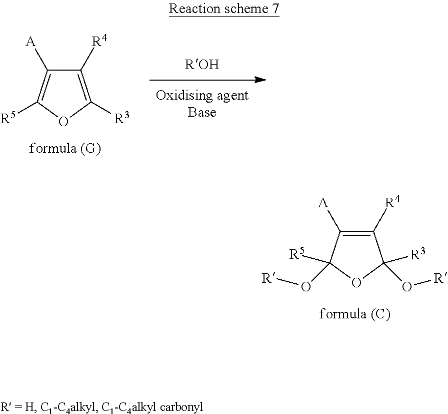

In another approach a compound of formula (I), wherein Q, Z, X, R¹, R², R³, R⁴, R⁵ and A are as defined for compounds of formula (I), may be prepared from a compound of formula (R) and an oxidant, in a suitable solvent at a suitable temperature, as outlined in reaction scheme 8. Example oxidants include, but are not limited to, 2,3-dichloro-5,6-dicyano-1,4-benzoquinone, tetrachloro-p-benzoquinone, potassium permanganate, manganese dioxide, 2,2,6,6-tetramethyl-1-piperidinyloxy and bromine. Related reactions are known in the literature.

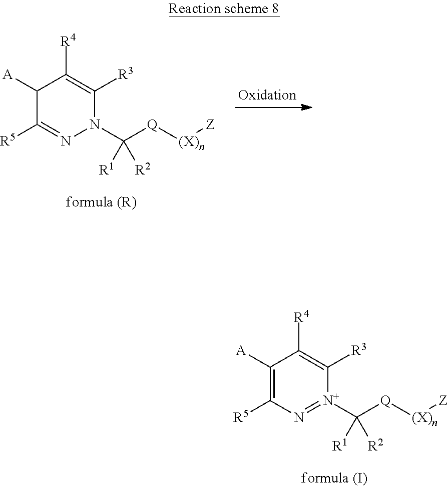

A compound of formula (R), wherein Q, Z, X, n, R¹, R², R³, R⁴, R⁵ and A are as defined for compounds of formula (I), may be prepared from a compound of formula (S), wherein Q, Z, X, n, R¹, R², R³, R⁴ and R⁵ are as defined for compounds of formula (I), and an organometallic of formula (T), wherein M′ includes, but is not limited to, organomagnesium, organolithium, organocopper and organozinc reagents, in a suitable solvent at a suitable temperature, optionally in the presence of an additional transition metal additive, as outlined in reaction scheme 9. Example conditions include treating a compound of formula (S) with a Grignard of formula (T), in the presence of 0.05-100 mol % copper iodide, in a solvent such as tetrahydrofuran at a temperature between −78° C. and 100° C. Organometallics of formula (T) are known in the literature, or may be prepared by known literature methods. Compounds of formula (S) may be prepared by analogous reactions to those for the preparation of compounds of formula (I) from a compound of formula (XX).

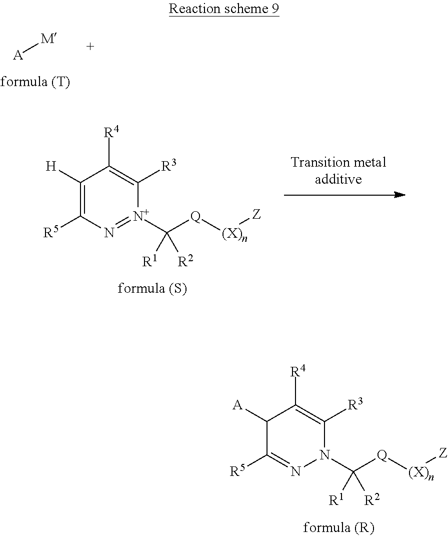

Biaryl pyridazines of formula (X) are known in the literature or may be prepared using literature methods. Example methods include, but are not limited to, the transition metal cross-coupling of compounds of formula (H) and formula (J), or alternatively compounds of formula (K) and formula (L), in which compounds of formula (J) and formula (L), wherein M′ is either an organostannane, organoboronic acid or ester, organotrifluoroborate, organomagnesium, organocopper or organozinc, as outlined in reaction scheme 10. Hal is defined as a halogen or pseudo halogen, for example triflate, mesylate and tosylate. Such cross-couplings include Stille, Suzuki-Miyaura, Negishi, and Kumada (for example WO 2017035409, WO 2016046530, WO 2015161924 and WO 2013062079). The coupling partners may be selected with reference to the specific cross-coupling reaction and target product. Transition metal catalysts, ligands, bases, solvents and temperatures may be selected with reference to the desired cross-coupling and are known in the literature. Compounds of formula (H), formula (K) and formula (L) are known in the literature, or may be prepared by known literature methods.

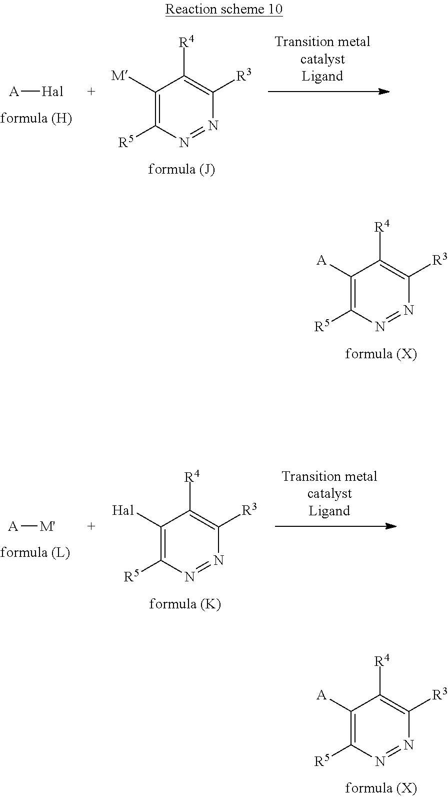

A compound of formula (J), wherein M′ is either an organostannane, organoboronic acid or ester, organotrifluoroborate, organomagnesium, organocopper or organozinc, may be prepared from a compound of formula (XX), wherein R³, R⁴ and R⁵ are as defined for compounds of formula (I), by metallation, as outlined in reaction scheme 11. Similar reactions are known in the literature (for example Ramphal et al, WO2015/153683, Unsinn et al., Organic Letters, 15(5), 1128-1131; 2013, Sadler et al., Organic & Biomolecular Chemistry, 12(37), 7318-7327; 2014). Alternatively, an organometallic of formula (J) may be prepared from compounds of formula (K), wherein R³, R⁴, R⁵ are as defined for compounds of formula (I), and Hal is defined as a halogen or pseudo halogen, for example triflate, mesylate and tosylate, as described in scheme 11. Example conditions to prepare a compound of formula (J) wherein M′ is an organostannane, include treatment of a compound of formula (K) with lithium tributyl tin in an appropriate solvent at an appropriate temperature (for example see WO 2010038465). Example conditions to prepare a compound of formula (J) wherein M′ is an organoboronic acid or ester, include treatment of a compound of formula (K) with bis(pinacolato)diboron, in the presence of an appropriate transition metal catalyst, appropriate ligand, appropriate base, in an appropriate solvent at an appropriate temperature (for example KR 2015135626). Compounds of formula (K) and formula (XX) are either known in the literature or can be prepared by known methods.

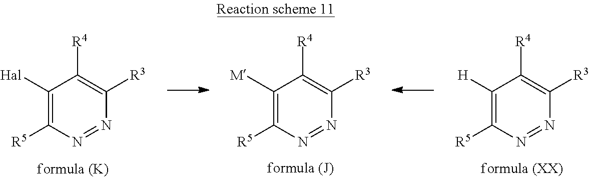

In another approach, a compound of formula (J), in which M′ is either an organostannane or organoboronic acid or ester, may be prepared from a compound of formula (N) and a compound of formula (O), wherein R³, R⁴ and R⁵ are as defined for compounds of formula (I), as outlined in reaction scheme 12. Examples of such a reaction are known in the literature, for example, Helm et al., Org. and Biomed. Chem., 2006, 4 (23), 4278, Sauer et al., Eur. J. Org. Chem., 1998, 12, 2885, and Helm, M. D.; Moore, J. E.; Plant, A.; Harrity, J. P. A., Angew. Chem. Int. Ed., 2005, 3889. Compounds of formula (N) and formula (O) are known in the literature.

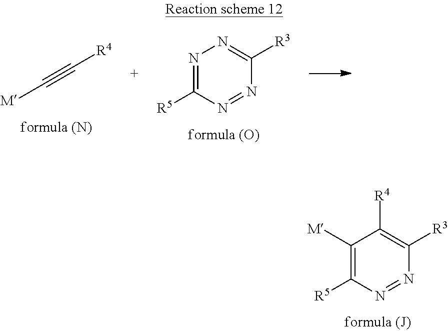

Compounds of formula (X), wherein R³, R⁴, R⁵ and A are as previously defined, may be prepared from compounds of formula (P) and formula (O), in an appropriate solvent, at an appropriate temperature, as outlined in reaction scheme 13. Examples of such a reaction are known in the literature, for example, WO 2001038332. Compounds of formula (P) are known in the literature, or may be prepared by known methods.

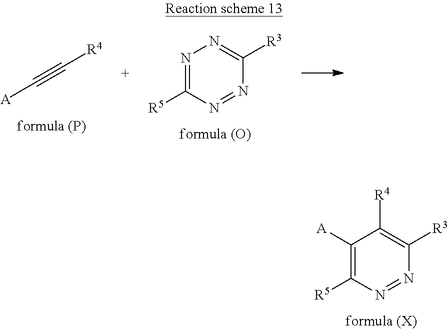

In a further approach a compound of formula (X), wherein R³, R⁴, R⁵ and A are as defined for compounds of formula (I), may be prepared from compounds of formula (C) and hydrazine, in an appropriate solvent, at an appropriate temperature, as outlined in reaction scheme 14. This reaction may also optionally be performed in the presence of an acid, for example aqueous sulfuric acid or aqueous hydrochloric acid. Similar reactions are known in the literature (for example DE 102005029094, and Chen, B.; Bohnert, T.; Zhou, X.; Dedon, P. C. Chem. Res. Toxicol., 2004, 1406). Compounds of formula (C) may be prepared as previously outlined.

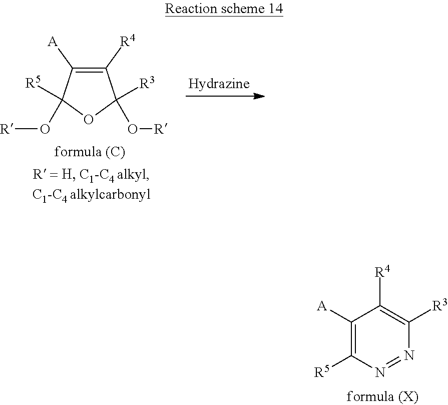

Finally, in an additional approach outlined in scheme 15, biaryl pyridazines of formula (X) may be prepared by classical ring synthesis approaches starting from a compound of formula (U), wherein T is a functional group which can be converted through one or more chemical steps into a 5-membered heteroaryl A, wherein A is as defined for compounds of formula (I). Such functional groups include, but are not limited to, acid, ester, nitrile, amide, thioamide and ketone. Related transformations are known in the literature.

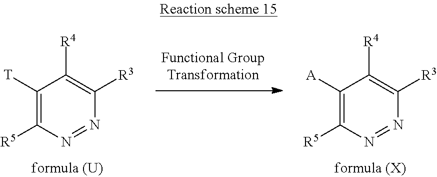

The compounds according to the invention can be used as herbicidal agents in unmodified form, but they are generally formulated into compositions in various ways using formulation adjuvants, such as carriers, solvents and surface-active substances. The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO and WHO Specifications for Pesticides, United Nations, First Edition, Second Revision (2010). Such formulations can either be used directly or diluted prior to use. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof.

The active ingredients can also be contained in very fine microcapsules. Microcapsules contain the active ingredients in a porous carrier. This enables the active ingredients to be released into the environment in controlled amounts (e.g. slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes can comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other polymers that are known to the person skilled in the art. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxypropanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol, propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like.

Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances.

A large number of surface-active substances can advantageously be used in both solid and liquid formulations, especially in those formulations which can be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecylbenzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in McCutcheon's Detergents and Emulsifiers Annual, MC Publishing Corp., Ridgewood N.J. (1981).

Further adjuvants that can be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and liquid and solid fertilisers.

The compositions according to the invention can include an additive comprising an oil of vegetable or animal origin, a mineral oil, alkyl esters of such oils or mixtures of such oils and oil derivatives. The amount of oil additive in the composition according to the invention is generally from 0.01 to 10%, based on the mixture to be applied. For example, the oil additive can be added to a spray tank in the desired concentration after a spray mixture has been prepared. Preferred oil additives comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow. Preferred oil additives comprise alkyl esters of C₈-C₂₂ fatty acids, especially the methyl derivatives of C₁₂-C₁₈ fatty acids, for example the methyl esters of lauric acid, palmitic acid and oleic acid (methyl laurate, methyl palmitate and methyl oleate, respectively). Many oil derivatives are known from the Compendium of Herbicide Adjuvants, 10^{th} Edition, Southern Illinois University, 2010.

The herbicidal compositions generally comprise from 0.1 to 99% by weight, especially from 0.1 to 95% by weight, compounds of formula (I) and from 1 to 99.9% by weight of a formulation adjuvant which preferably includes from 0 to 25% by weight of a surface-active substance. The inventive compositions generally comprise from 0.1 to 99% by weight, especially from 0.1 to 95% by weight, of compounds of the present invention and from 1 to 99.9% by weight of a formulation adjuvant which preferably includes from 0 to 25% by weight of a surface-active substance. Whereas commercial products may preferably be formulated as concentrates, the end user will normally employ dilute formulations.

The rates of application vary within wide limits and depend on the nature of the soil, the method of application, the crop plant, the pest to be controlled, the prevailing climatic conditions, and other factors governed by the method of application, the time of application and the target crop. As a general guideline compounds may be applied at a rate of from 1 to 2000 I/ha, especially from 10 to 1000 I/ha.

Preferred formulations can have the following compositions (weight %):

Emulsifiable Concentrates:

active ingredient: 1 to 95%, preferably 60 to 90%

surface-active agent: 1 to 30%, preferably 5 to 20%

liquid carrier: 1 to 80%, preferably 1 to 35%

Dusts:

active ingredient: 0.1 to 10%, preferably 0.1 to 5%

solid carrier: 99.9 to 90%, preferably 99.9 to 99%

Suspension Concentrates:

active ingredient: 5 to 75%, preferably 10 to 50%

water: 94 to 24%, preferably 88 to 30%

surface-active agent: 1 to 40%, preferably 2 to 30%

Wettable Powders:

active ingredient: 0.5 to 90%, preferably 1 to 80%

surface-active agent: 0.5 to 20%, preferably 1 to 15%

solid carrier: 5 to 95%, preferably 15 to 90%

Granules:

active ingredient: 0.1 to 30%, preferably 0.1 to 15%

solid carrier: 99.5 to 70%, preferably 97 to 85%

The composition of the present may further comprise at least one additional pesticide. For example, the compounds according to the invention can also be used in combination with other herbicides or plant growth regulators. In a preferred embodiment the additional pesticide is a herbicide and/or herbicide safener.

Thus, compounds of formula (I) can be used in combination with one or more other herbicides to provide various herbicidal mixtures. Specific examples of such mixtures include (wherein “I” represents a compound of formula (I)):—I+acetochlor; I+acifluorfen (including acifluorfen-sodium); I+aclonifen; I+alachlor; I+alloxydim; I+ametryn; I+amicarbazone; I+amidosulfuron; I+aminocyclopyrachlor; I+aminopyralid; I+amitrole; I+asulam; I+atrazine; I+bensulfuron (including bensulfuron-methyl); I+bentazone; I+bicyclopyrone; I+bilanafos; I+bifenox; I+bispyribac-sodium; I+bixlozone; I+bromacil; I+bromoxynil; I+butachlor; I+butafenacil; I+cafenstrole; I+carfentrazone (including carfentrazone-ethyl); I+cloransulam (including cloransulam-methyl); I+chlorimuron (including chlorimuron-ethyl); I+chlorotoluron; I+cinosulfuron; I+chlorsulfuron; I+cinmethylin; I+clacyfos; I+clethodim; I+clodinafop (including clodinafop-propargyl); I+clomazone; I+clopyralid; I+cyclopyranil; I+cyclopyrimorate; I+cyclosulfamuron; I+cyhalofop (including cyhalofop-butyl); I+2,4-D (including the choline salt and 2-ethylhexyl ester thereof); I+2,4-DB; I+daimuron; I+desmedipham; I+dicamba (including the aluminum, aminopropyl, bis-aminopropylmethyl, choline, dichloroprop, diglycolamine, dimethylamine, dimethylammonium, potassium and sodium salts thereof); I+diclofop-methyl; I+diclosulam; I+diflufenican; I+difenzoquat; I+diflufenican; I+diflufenzopyr; I+dimethachlor; I+dimethenamid-P; I+diquat dibromide; I+diuron; I+esprocarb; I+ethalfluralin; I+ethofumesate; I+fenoxaprop (including fenoxaprop-P-ethyl); I+fenoxasulfone; I+fenquinotrione; I+fentrazamide; I+flazasulfuron; I+florasulam; I+florpyrauxifen; I+fluazifop (including fluazifop-P-butyl); I+flucarbazone (including flucarbazone-sodium); I+flufenacet; I+flumetralin; I+flumetsulam; I+flumioxazin; I+flupyrsulfuron (including flupyrsulfuron-methyl-sodium); I+fluroxypyr (including fluroxypyr-meptyl); I+fluthiacet-methyl; I+fomesafen; I+foramsulfuron; I+glufosinate (including the ammonium salt thereof); I+glyphosate (including the diammonium, isopropylammonium and potassium salts thereof); I+halauxifen (including halauxifen-methyl); I+halosulfuron-methyl; I+haloxyfop (including haloxyfop-methyl); I+hexazinone; I+hydantocidin; I+imazamox; I+imazapic; I+imazapyr; I+imazaquin; I+imazethapyr; I+indaziflam; I+iodosulfuron (including iodosulfuron-methyl-sodium); I+iofensulfuron; I+iofensulfuron-sodium; I+ioxynil; I+isoproturon; I+ipfencarbazone; I+isoxaben; I+isoxaflutole; I+lactofen; I+lancotrione; I+linuron; I+MCPA; I+MCPB; I+mecoprop-P; I+mefenacet; I+mesosulfuron; I+mesosulfuron-methyl; I+mesotrione; I+metamitron; I+metazachlor; I+methiozolin; I+metobromuron; I+metolachlor; I+metosulam; I+metoxuron; I+metribuzin; I+metsulfuron; I+molinate; I+napropamide; I+nicosulfuron; I+norflurazon; I+orthosulfamuron; I+oxadiargyl; I+oxadiazon; I+oxasulfuron; I+oxyfluorfen; I+paraquat dichloride; I+pendimethalin; I+penoxsulam; I+phenmedipham; I+picloram; I+picolinafen; I+pinoxaden; I+pretilachlor; I+primisulfuron-methyl; I+prodiamine; I+prometryn; I+propachlor; I+propanil; I+propaquizafop; I+propham; I+propyrisulfuron, I+propyzamide; I+prosulfocarb; I+prosulfuron; I+pyraclonil; I+pyraflufen (including pyraflufen-ethyl); I+pyrasulfotole; I+pyrazolynate, I+pyrazosulfuron-ethyl; I+pyribenzoxim; I+pyridate; I+pyriftalid; I+pyrimisulfan; I+pyrithiobac-sodium; I+pyroxasulfone; I+pyroxsulam; I+quinclorac; I+quinmerac; I+quizalofop (including quizalofop-P-ethyl and quizalofop-P-tefuryl); I+rimsulfuron; I+saflufenacil; I+sethoxydim; I+simazine; I+S-metolachlor; I+sulcotrione; I+sulfentrazone; I+sulfosulfuron; I+tebuthiuron; I+tefuryltrione; I+tembotrione; I+terbuthylazine; I+terbutryn; I+thiencarbazone; I+thifensulfuron; I+tiafenacil; I+tolpyralate; I+topramezone; I+tralkoxydim; I+triafamone; I+triallate; I+triasulfuron; I+tribenuron (including tribenuron-methyl); I+triclopyr; I+trifloxysulfuron (including trifloxysulfuron-sodium); I+trifludimoxazin; I+trifluralin; I+triflusulfuron; I+tritosulfuron; I+4-hydroxy-1-methoxy-5-methyl-3-[4-(trifluoromethyl)-2-pyridyl]imidazolidin-2-one; I+4-hydroxy-1,5-dimethyl-3-[4-(trifluoromethyl)-2-pyridyl]imidazolidin-2-one; I+5-ethoxy-4-hydroxy-1-methyl-3-[4-(trifluoromethyl)-2-pyridyl]imidazolidin-2-one; I+4-hydroxy-1-methyl-3-[4-(trifluoromethyl)-2-pyridyl]imidazolidin-2-one; I+4-hydroxy-1,5-dimethyl-3-[1-methyl-5-(trifluoromethyl)pyrazol-3-yl]imidazolidin-2-one; I+(4R)1-(5-tert-butylisoxazol-3-yl)-4-ethoxy-5-hydroxy-3-methyl-imidazolidin-2-one; I+3-[2-(3,4-dimethoxyphenyl)-6-methyl-3-oxo-pyridazine-4-carbonyl]bicyclo[3.2.1]octane-2,4-dione; I+2-[2-(3,4-dimethoxyphenyl)-6-methyl-3-oxo-pyridazine-4-carbonyl]-5-methyl-cyclohexane-1,3-dione; I+2-[2-(3,4-dimethoxyphenyl)-6-methyl-3-oxo-pyridazine-4-carbonyl]cyclohexane-1,3-dione; I+2-[2-(3,4-dimethoxyphenyl)-6-methyl-3-oxo-pyridazine-4-carbonyl]-5,5-dimethyl-cyclohexane-1,3-dione; I+6-[2-(3,4-dimethoxyphenyl)-6-methyl-3-oxo-pyridazine-4-carbonyl]-2,2,4,4-tetramethyl-cyclohexane-1,3,5-trione; I+2-[2-(3,4-dimethoxyphenyl)-6-methyl-3-oxo-pyridazine-4-carbonyl]-5-ethyl-cyclohexane-1,3-dione; I+2-[2-(3,4-dimethoxyphenyl)-6-methyl-3-oxo-pyridazine-4-carbonyl]-4,4,6,6-tetramethyl-cyclohexane-1,3-dione; I+2-[6-cyclopropyl-2-(3,4-dimethoxyphenyl)-3-oxo-pyridazine-4-carbonyl]-5-methyl-cyclohexane-1,3-dione; I+3-[6-cyclopropyl-2-(3,4-dimethoxyphenyl)-3-oxo-pyridazine-4-carbonyl]bicyclo[3.2.1]octane-2,4-dione; I+2-5 [6-cyclopropyl-2-(3,4-dimethoxyphenyl)-3-oxo-pyridazine-4-carbonyl]-5,5-dimethyl-cyclohexane-1,3-dione; I+6-[6-cyclopropyl-2-(3,4-dimethoxyphenyl)-3-oxo-pyridazine-4-carbonyl]-2,2,4,4-tetramethyl-cyclohexane-1,3,5-trione; I+2-[6-cyclopropyl-2-(3,4-dimethoxyphenyl)-3-oxo-pyridazine-4-carbonyl]cyclohexane-1,3-dione; I+4-[2-(3,4-dimethoxyphenyl)-6-methyl-3-oxo-pyridazine-4-carbonyl]-2,2,6,6-tetramethyl-tetrahydropyran-3,5-dione and I+4-[6-cyclopropyl-2-(3,4-dimethoxyphenyl)-3-oxo-pyridazine-4-carbonyl]-2,2,6,6-tetramethyl-tetrahydropyran-3,5-dione.

Especially preferred examples of such mixtures include:—I+ametryn; I+atrazine; I+bicyclopyrone; I+butafenacil; I+chlorotoluron; I+clodinafop-propargyl; I+clomazone; I+2,4-D (including the choline salt and 2-ethylhexyl ester thereof); I+dicamba (including the aluminum, aminopropyl, bis-aminopropylmethyl, choline, diglycolamine, dimethylamine, dimethylammonium, potassium and sodium salts thereof); I+dimethachlor; I+diquat dibromide; I+fluazifop-P-butyl; I+flumetralin; I+fomesafen; I+glufosinate-ammonium; I+glyphosate (including the diammonium, isopropylammonium and potassium salts thereof); I+mesotrione; I+molinate; I+napropamide; I+nicosulfuron; I+paraquat dichloride; I+pinoxaden; I+pretilachlor; I+primisulfuron-methyl; I+prometryn; I+prosulfocarb; I+prosulfuron; I+pyridate; I+pyriftalid; I+pyrazolynate, I+S-metolachlor; I+terbuthylazine; I+terbutryn; I+tralkoxydim; I+triasulfuron and I+trifloxysulfuron-sodium.

Preferred herbicide mixture products for weed control in cereals (especially wheat and/or barley) include:—I+amidosulfuron; I+aminopyralid; I+bromoxynil; I+carfentrazone-ethyl; I+chlorotoluron; I+clodinafop-propargyl; I+clopyralid; I+2,4-D (including the choline salt and 2-ethylhexyl ester thereof); I+dicamba (including the aluminum, aminopropyl, bis-aminopropylmethyl, choline, diglycolamine, dimethylamine, dimethylammonium, potassium and sodium salts thereof); I+difenzoquat; I+diflufenican; I+fenoxaprop-P-ethyl; I+florasulam; I+flucarbazone-sodium; I+flufenacet; flupyrsulfuron-methyl-sodium; I+fluroxypyr-meptyl; I+halauxifen-methyl; I+iodosulfuron-methyl-sodium; I+iofensulfuron; I+iofensulfuron-sodium; I+mesosulfuron; I+mesosulfuron-methyl; I+metsulfuron; I+pendimethalin; I+pinoxaden; I+prosulfocarb; I+pyrasulfotole; I+pyroxasulfone; I+pyroxsulam; I+topramezone; I+tralkoxydim; I+triasulfuron and I+tribenuron-methyl.

Preferred herbicide mixture products for weed control in corn include:—I+acetochlor; I+alachlor; I+atrazine; I+bicyclopyrone; I+2,4-D (including the choline salt and 2-ethylhexyl ester thereof); I+dicamba (including the aluminum, aminopropyl, bis-aminopropylmethyl, choline, diglycolamine, dimethylamine, dimethylammonium, potassium and sodium salts thereof); I+diflufenzopyr; I+dimethenamid-P; I+flumioxazin; I+fluthiacet-methyl; I+foramsulfuron; I+glufosinate (including the ammonium salt thereof); I+glyphosate (including the diammonium, isopropylammonium and potassium salts thereof); I+isoxaflutole; I+mesotrione; I+nicosulfuron; I+primisulfuron-methyl; I+prosulfuron; I+pyroxasulfone; I+rimsulfuron; I+S-metolachlor, I+terbutylazine; I+tembotrione; I+thiencarbazone and I+thifensulfuron.

Preferred herbicide mixture products for weed control in rice include:—I+2,4-D; I+2,4-D choline salt; I+2,4-D-2-ethylhexyl ester; I+bensulfuron-methyl; I+bispyribac-sodium; I+cafenstrole; I+cinosulfuron; I+clomazone; I+cyhalofop-butyl; I+daimuron; I+dicamba (including the aluminum, aminopropyl, bis-aminopropylmethyl, choline, diglycolamine, dimethylamine, dimethylammonium, potassium and sodium salts thereof); I+esprocarb; I+fenoxaprop-P-ethyl; I+florasulam; I+halauxifen-methyl; I+halosulfuron-methyl; I+iofensulfuron; I+ipfencarbazone; I+mefenacet; I+mesotrione; I+metsulfuron; I+molinate; I+orthosulfamuron; I+oxadiargyl; I+oxadiazon; I+pendimethalin; I+penoxsulam; I+pretilachlor; I+pyrazolynate, I+pyrazosulfuron-ethyl; I+pyribenzoxim; I+pyriftalid; I+quinclorac; I+tefuryltrione; I+triafamone and I+triasulfuron.

Preferred herbicide mixtures for weed control in soybean include:—I+acifluorfen-sodium; I+ametryn; I+atrazine; I+bentazone; I+bicyclopyrone; I+bromoxynil; I+carfentrazone-ethyl; I+chlorimuron-ethyl; I+clethodim; I+clomazone; I+2,4-D (including the choline salt and 2-ethylhexyl ester thereof); I+dicamba (including the aluminum, aminopropyl, bis-aminopropylmethyl, choline, diglycolamine, dimethylamine, dimethylammonium, potassium and sodium salts thereof); I+diquat dibromide; I+diuron; I+fenoxaprop-P-ethyl; I+fluazifop-P-butyl; I+flufenacet; I+flumioxazin; I+fomesafen; I+glufosinate (including the ammonium salt thereof); I+glyphosate (including the diammonium, isopropylammonium and potassium salts thereof); I+imazethapyr; I+lactofen; I+mesotrione; I+metolachlor; I+metribuzin; I+nicosulfuron; I+oxyfluorfen; I+paraquat dichloride; I+pendimethalin; I+pyroxasulfone; I+quizalofop-P-ethyl; I+saflufenacil; I+sethoxydim; I+S-metolachlor and I+sulfentrazone.

The mixing partners of the compound of formula (I) may also be in the form of esters or salts, as mentioned e.g. in The Pesticide Manual, Fourteenth Edition, British Crop Protection Council, 2006.

The compound of formula (I) can also be used in mixtures with other agrochemicals such as fungicides, nematicides or insecticides, examples of which are given in The Pesticide Manual.

The mixing ratio of the compound of formula (I) to the mixing partner is preferably from 1:100 to 1000:1.

The mixtures can advantageously be used in the above-mentioned formulations (in which case “active ingredient” relates to the respective mixture of compound of formula (I) with the mixing partner).

Compounds of formula (I) of the present invention may also be combined with herbicide safeners. Preferred combinations (wherein “I” represents a compound of formula (I)) include:—I+benoxacor, I+cloquintocet (including cloquintocet-mexyl); I+cyprosulfamide; I+dichlormid; I+fenchlorazole (including fenchlorazole-ethyl); I+fenclorim; I+fluxofenim; I+furilazole; I+isoxadifen (including isoxadifen-ethyl); I+mefenpyr (including mefenpyr-diethyl); I+metcamifen; I+N-(2-methoxybenzoyl)-4-[(methylaminocarbonyl)amino] benzenesulfonamide and I+oxabetrinil.

Particularly preferred are mixtures of a compound of formula (I) with cyprosulfamide, isoxadifen (including isoxadifen-ethyl), cloquintocet (including cloquintocet-mexyl) and/or N-(2-methoxybenzoyl)-4-[(methyl-aminocarbonyl)amino]benzenesulfonamide.

The safeners of the compound of formula (I) may also be in the form of esters or salts, as mentioned e.g. in The Pesticide Manual, 14^{th} Edition (BCPC), 2006. The reference to cloquintocet-mexyl also applies to a lithium, sodium, potassium, calcium, magnesium, aluminium, iron, ammonium, quaternary ammonium, sulfonium or phosphonium salt thereof as disclosed in WO 02/34048, and the reference to fenchlorazole-ethyl also applies to fenchlorazole, etc.

Preferably the mixing ratio of compound of formula (I) to safener is from 100:1 to 1:10, especially from 20:1 to 1:1.

The mixtures can advantageously be used in the above-mentioned formulations (in which case “active ingredient” relates to the respective mixture of compound of formula (I) with the safener).

The compounds of formula (I) of this invention are useful as herbicides. The present invention therefore further comprises a method for controlling unwanted plants comprising applying to the said plants or a locus comprising them, an effective amount of a compound of the invention or a herbicidal composition containing said compound. ‘Controlling’ means killing, reducing or retarding growth or preventing or reducing germination. Generally the plants to be controlled are unwanted plants (weeds). ‘Locus’ means the area in which the plants are growing or will grow.

The rates of application of compounds of formula (I) may vary within wide limits and depend on the nature of the soil, the method of application (pre-emergence; post-emergence; application to the seed furrow; no tillage application etc.), the crop plant, the weed(s) to be controlled, the prevailing climatic conditions, and other factors governed by the method of application, the time of application and the target crop. The compounds of formula (I) according to the invention are generally applied at a rate of from 10 to 2000 g/ha, especially from 50 to 1000 g/ha.

The application is generally made by spraying the composition, typically by tractor mounted sprayer for large areas, but other methods such as dusting (for powders), drip or drench can also be used.

Useful plants in which the composition according to the invention can be used include crops such as cereals, for example barley and wheat, cotton, oilseed rape, sunflower, maize, rice, soybeans, sugar beet, sugar cane and turf.

Crop plants can also include trees, such as fruit trees, palm trees, coconut trees or other nuts. Also included are vines such as grapes, fruit bushes, fruit plants and vegetables.

Crops are to be understood as also including those crops which have been rendered tolerant to herbicides or classes of herbicides (e.g. ALS-, GS-, EPSPS-, PPO-, ACCase- and HPPD-inhibitors) by conventional methods of breeding or by genetic engineering. An example of a crop that has been rendered tolerant to imidazolinones, e.g. imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®.

Crops are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). The Bt toxin is a protein that is formed naturally by Bacillus thuringiensis soil bacteria. Examples of toxins, or transgenic plants able to synthesise such toxins, are described in EP-A-451 878, EP-A-374 753, WO 93/07278, WO 95/34656, WO 03/052073 and EP-A-427 529. Examples of transgenic plants comprising one or more genes that code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®. Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crops are also to be understood to include those which are obtained by conventional methods of breeding or genetic engineering and contain so-called output traits (e.g. improved storage stability, higher nutritional value and improved flavour).

Other useful plants include turf grass for example in golf-courses, lawns, parks and roadsides, or grown commercially for sod, and ornamental plants such as flowers or bushes.

Compounds of formula (I) and compositions of the invention can typically be used to control a wide variety of monocotyledonous and dicotyledonous weed species. Examples of monocotyledonous species that can typically be controlled include Alopecurus myosuroides, Avena fatua, Brachiaria plantaginea, Bromus tectorum, Cyperus esculentus, Digitaria sanguinalis, Echinochloa crus-galli, Lolium perenne, Lolium multiflorum, Panicum miliaceum, Poa annua, Setaria viridis, Setaria faberi and Sorghum bicolor. Examples of dicotyledonous species that can be controlled include Abutilon theophrasti, Amaranthus retroflexus, Bidens pilosa, Chenopodium album, Euphorbia heterophylla, Galium aparine, Ipomoea hederacea, Kochia scoparia, Polygonum convolvulus, Sida spinosa, Sinapis arvensis, Solanum nigrum, Stellaria media, Veronica persica and Xanthium strumarium.

The compounds of formula (I) are also useful for pre-harvest desiccation in crops, for example, but not limited to, potatoes, soybean, sunflowers and cotton. Pre-harvest desiccation is a well-known process used to desiccate crop foliage without significant damage to the crop itself to aid harvesting. Compounds/compositions of the invention are particularly useful in non-selective burn-down applications, and as such may also be used to control volunteer or escape crop plants.

Various aspects and embodiments of the present invention will now be illustrated in more detail by way of example. It will be appreciated that modification of detail may be made without departing from the scope of the invention.

### EXAMPLES

The Examples which follow serve to illustrate, but do not limit, the invention.

### Formulation Examples

| Wettable powders | a) | b) | c) |
|---|---|---|---|
| active ingredients | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The combination is thoroughly mixed with the adjuvants and the mixture is thoroughly ground in a suitable mill, affording wettable powders that can be diluted with water to give suspensions of the desired concentration.

Emulsifiable concentrate

| | |
|---|---|
| active ingredients | 10% |
| octylphenol polyethylene glycol ether | 3% |
| (4-5 mol of ethylene oxide) | |
| calcium dodecylbenzenesulfonate | 3% |
| castor oil polyglycol ether (35 mol of ethylene oxide) | 4% |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredients | 5% | 6% | 4% |
| Talcum | 95% | — | — |
| Kaolin | — | 94% | — |
| mineral filler | — | — | 96% |

Ready-for-use dusts are obtained by mixing the combination with the carrier and grinding the mixture in a suitable mill.

Extruded granules

| | |
|---|---|
| Active ingredients | 15% |
| sodium lignosulfonate | 2% |
| carboxymethylcellu lose | 1% |
| Kaolin | 82% |

The combination is mixed and ground with the adjuvants, and the mixture is moistened with water. The mixture is extruded and then dried in a stream of air.

Coated granules

| | |
|---|---|
| Active ingredients | 8% |
| polyethylene glycol (mol. wt. 200) | 3% |
| Kaolin | 89% |

The finely ground combination is uniformly applied, in a mixer, to the kaolin moistened with polyethylene glycol. Non-dusty coated granules are obtained in this manner.

Suspension concentrate

| | |
|---|---|
| active ingredients | 40% |
| propylene glycol | 10% |
| nonylphenol polyethylene glycol ether | 6% |
| (15 mol of ethylene oxide) | |
| Sodium lignosulfonate | 10% |
| carboxymethylcellulose | 1% |
| silicone oil (in the form of a 75% | 1% |
| emulsion in water) | |
| Water | 32% |

The finely ground combination is intimately mixed with the adjuvants, giving a suspension concentrate from which suspensions of any desired dilution can be obtained by dilution with water.

Slow Release Capsule Suspension

28 parts of the combination are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed.

The obtained capsule suspension is stabilized by adding 0.25 parts of a thickener and 3 parts of a dispersing agent. The capsule suspension formulation contains 28% of the active ingredients. The medium capsule diameter is 8-15 microns.

The resulting formulation is applied to seeds as an aqueous suspension in an apparatus suitable for that purpose.

### LIST OF ABBREVIATIONS

Boc=tert-butyloxycarbonyl

br=broad

CDCl₃=chloroform-d

CD₃OD=methanol-d

° C.=degrees Celsius

D₂O=water-d

DCM=dichloromethane

d=doublet

dd=double doublet

dt=double triplet

DMSO=dimethylsulfoxide

EtOAc=ethyl acetate

h=hour(s)

HCl=hydrochloric acid

HPLC=high-performance liquid chromatography (description of the apparatus and the methods used for HPLC are given below)

m=multiplet

M=molar

min=minutes

MHz=megahertz

mL=millilitre

mp=melting point

ppm=parts per million

q=quartet

quin=quintet

rt=room temperature

s=singlet

t=triplet

THE=tetrahydrofuran

LC/MS=Liquid Chromatography Mass Spectrometry (description of the apparatus and the methods used for LC/MS analysis are given below)

Preparative Reverse Phase HPLC Method:

Compounds purified by mass directed preparative HPLC using ES+/ES− on a Waters FractionLynx Autopurification system comprising a 2767 injector/collector with a 2545 gradient pump, two 515 isocratic pumps, SFO, 2998 photodiode array (Wavelength range (nm): 210 to 400), 2424 ELSD and QDa mass spectrometer. A Waters Atlantis T3 5 micron 19×10 mm guard column was used with a Waters Atlantis T3 OBD, 5 micron 30×100 mm prep column.

Ionisation method: Electrospray positive and negative: Cone (V) 20.00, Source Temperature (° C.) 120, Cone Gas Flow (L/Hr.) 50

Mass range (Da): positive 100 to 800, negative 115 to 800.

The preparative HPLC was conducted using an 11.4 minute run time (not using at column dilution, bypassed with the column selector), according to the following gradient table:

| Time (mins) | Solvent A (%) | Solvent B (%) | Flow (ml/min) |
|---|---|---|---|
| 0.00 | 100 | 0 | 35 |
| 2.00 | 100 | 0 | 35 |
| 2.01 | 100 | 0 | 35 |
| 7.0 | 90 | 10 | 35 |
| 7.3 | 0 | 100 | 35 |
| 9.2 | 0 | 100 | 35 |
| 9.8 | 99 | 1 | 35 |
| 11.35 | 99 | 1 | 35 |
| 11.40 | 99 | 1 | 35 |

515 pump 0 ml/min Acetonitrile (ACD)

515 pump 1 ml/min 90% Methanol/10% Water (makeup pump)

Solvent A: Water with 0.05% Trifluoroacetic Acid

Solvent B: Acetonitrile with 0.05% Trifluoroacetic Acid

### Preparation Examples

### Example 1: Preparation of 2-(4-thiazol-2-ylpyridazin-1-ium-1-yl)ethanesulfonate A-1

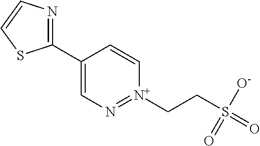 [SMILES: O=S(=O)([O-])CC[n+]1ccc(-c2nccs2)cn1]

### Step 1: Preparation of 2-pyridazin-4-ylthiazole

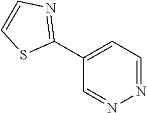 [SMILES: c1cc(-c2nccs2)cnn1]

To a mixture of 2-bromothiazole (68 mg) and 4-(4,4,5,5-tetramethyl-1,3,2-dioxaborolan-2-yl)pyridazine (86 mg) in N,N-dimethylformamide (1 mL) was added aqueous 2M sodium carbonate (0.4 mL), followed by degassing and purging with nitrogen for ten minutes.

Chloro(crotyl)(tricyclohexylphosphine)palladium(II) (40 mg) was added and the reaction mixture was degassed once again. This mixture was heated at 100° C. under microwave irradiation for 30 minutes. After cooling to room temperature the reaction mixture was concentrated and purified by preparative reverse phase HPLC to afford 2-pyridazin-4-ylthiazole as a cream solid.

¹H NMR (400 MHz, CDCl₃) 9.75 (dd, 1H) 9.31 (dd, 1H) 8.06 (d, 1H) 7.96 (dd, 1H) 7.60 (d, 1H)

### Step 2: Preparation of 2-(4-thiazol-2-ylpyridazin-1-ium-1-yl)ethanesulfonate A-1

A mixture of 2-pyridazin-4-ylthiazole (40 mg) and sodium 2-bromoethanesulfonate (58 mg) was heated in water (1 mL) at 100° C. for 44 hours. The reaction mixture was cooled and washed with dichloromethane. The aqueous phase was concentrated and purified by preparative reverse phase HPLC to afford 2-(4-thiazol-2-ylpyridazin-1-ium-1-yl)ethanesulfonate as a white solid.

¹H NMR (400 MHz, D₂O) 9.84-9.94 (m, 1H) 9.63-9.72 (m, 1H) 8.82 (dd, 1H) 8.14-8.25 (m, 1H) 8.08 (d, 1H) 5.09-5.19 (m, 2H) 3.54-3.68 (m, 2H)

### Example 2: Preparation of 4-(1-methylpyrazol-3-yl)pyridazine

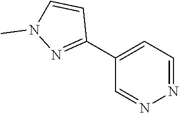

To a mixture of 3-bromo-1-methyl-pyrazole (156 mg) and 4-(4,4,5,5-tetramethyl-1,3,2-dioxaborolan-2-yl)pyridazine (200 mg) in 1,4-dioxane (2 mL) was added potassium phosphate (0.5 g) and water (0.4 mL), followed by degassing and purging with nitrogen for 10 minutes.

Chloro(crotyl)(tricyclohexylphosphine)palladium(II) (28 mg) was added and the reaction mixture was degassed once again. This mixture was heated at 110° C. under microwave irradiation for 30 minutes. After cooling to room temperature the reaction mixture was concentrated and purified by preparative reverse phase HPLC to afford 4-(1-methylpyrazol-3-yl)pyridazine as a white solid.

¹H NMR (400 MHz, CD₃OD) 9.77 (dd, 1H) 9.33 (dd, 1H) 8.41 (dd, 1H) 7.80 (d, 1H) 7.10 (d, 1H) 4.04 (s, 3H)

### Example 3: Preparation of 3-(4-oxazol-2-ylpyridazin-1-ium-1-yl)propane-1-sulfonate A-3

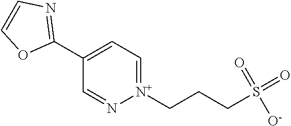 [SMILES: O=S(=O)([O-])CCC[n+]1ccc(-c2ncco2)cn1]

### Step 1: Preparation of 2-pyridazin-4-yloxazole

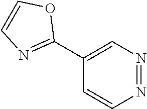 [SMILES: c1cc(-c2ncco2)cnn1]

To a mixture of tributyl(oxazol-2-yl)stannane (1 g), 4-bromopyridazine (0.4 g), palladium (0) tetrakis(triphenylphosphine) (0.291 g), cesium fluoride (0.382 g) and cuprous iodide (0.019 g) was added 1,4-dioxane (10 mL). This mixture was heated at 140° C. under microwave irradiation for 60 minutes. The reaction mixture was concentrated and purified by preparative reverse phase HPLC to afford 2-pyridazin-4-yloxazole as a beige solid.

¹H NMR (400 MHz, D₂O) 9.59 (dd, 1H) 9.24 (dd, 1H) 8.15 (dd, 1H) 8.03 (d, 1H) 7.37 (d, 1H)

### Step 2: Preparation of 3-(4-oxazol-2-ylpyridazin-1-ium-1-yl)propane-1-sulfonate A-3

To a mixture of 2-pyridazin-4-yloxazole (30 mg) in 1,4-dioxane (1 mL) was added 1,3-propanesultone (30 mg). The mixture was heated at 90° C. for 44 hours. The resulting precipitate was filtered off, washed with acetone and purified by preparative reverse phase HPLC to afford 3-(4-oxazol-2-ylpyridazin-1-ium-1-yl)propane-1-sulfonate as a white solid.

¹H NMR (400 MHz, D₂O) 9.83-9.95 (m, 1H) 9.73 (d, 1H) 8.86 (dd, 1H) 8.08-8.31 (m, 1H) 7.49-7.71 (m, 1H) 4.85-5.08 (m, 2H) 2.85-3.16 (m, 2H) 2.50 (quin, 2H)

### Example 4: Preparation of 2-pyridazin-4-yl-1,3,4-oxadiazole

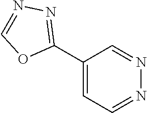 [SMILES: c1cc(-c2nnco2)cnn1]

### Step 1: Preparation of pyridazine-4-carbohydrazide

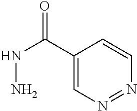

To a solution of methyl pyridazine-4-carboxylate (0.4 g) in methanol (4.92 mL) was added hydrazine hydrate (1.16 g) and the mixture was heated at reflux overnight. The reaction mixture was cooled and concentrated to afford pyridazine-4-carbohydrazide as a brown solid.

¹H NMR (400 MHz, CD₃OD) 9.52-9.48 (m, 1H) 9.36 (dd, 1H) 8.00 (dd, 1H) (three NH protons missing)

### Step 2: Preparation of 2-pyridazin-4-yl-1,3,4-oxadiazole

A mixture of pyridazine-4-carbohydrazide (0.370 g) and trimethoxymethane (7.8 g) was heated at reflux overnight. The reaction mixture was cooled, concentrated and purified by silica gel chromatography eluting with 0 to 50% acetonitrile in dichloromethane to afford 2-pyridazin-4-yl-1,3,4-oxadiazole as a yellow solid.

¹H NMR (400 MHz, CDCl₃) 9.87-9.84 (dd, 1H) 9.50-9.46 (dd, 1H) 8.66 (s, 1H) 8.10 (dd, 1H)

### Example 5: Preparation of 5-pyridazin-4-y-1,2,4-thiadiazole

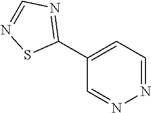 [SMILES: c1cc(-c2ncns2)cnn1]

### Step 1: Preparation of pyridazine-4-carbothioamide

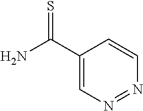

To a solution of pyridazine-4-carbonitrile (0.5 g) in methanolic ammonia (2M solution, 5 mL) was added phosphorus pentasulfide (1.06 g), keeping the reaction temperature below 35° C. After stirring at room temperature overnight water was added. The reaction mixture was cooled and the resulting precipitate removed by filtration. The aqueous phase was washed with dichloromethane and the organic phase discarded. The aqueous phase was left to stand at room temperature for several days and the resulting solid was again removed by filtration. The combined solids were shown to be the desired compound pyridazine-4-carbothioamide.

¹H NMR (400 MHz, CD₃OD) 9.53 (dd, 1H) 9.26 (dd, 1H) 7.94 (dd, 1H) (two NH protons missing)

### Step 2: Preparation of N,N-(dimethylaminomethylene)pyridazine-4-carbothioamide

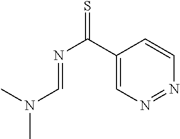

Pyridazine-4-carbothioamide (1.46 g) and 1,1-dimethoxy-N,N-dimethyl-methanamine (1.4 mL) were stirred together at room temperature for 6 hours. The reaction was concentrated and purified by silica gel chromatography eluting with 0 to 50% methanol in acetonitrile to afford N,N-(dimethylaminomethylene)pyridazine-4-carbothioamide as a dark red solid.

¹H NMR (400 MHz, CDCl₃) 9.94 (dd, 1H) 9.27 (dd, 1H) 8.81 (s, 1H) 8.27 (dd, 1H) 3.38-3.32 (m, 6H)

### Step 3: Preparation of 5-pyridazin-4-yl-1,2,4-thiadiazole

To a mixture of N,N-(dimethylaminomethylene)pyridazine-4-carbothioamide (1 g), pyridine (0.83 mL) and ethanol (25 mL) at room temperature was added a solution of hydroxylamine-O-sulfonic acid (640 mg) in methanol (10 mL). After 2 hours the reaction mixture was partitioned between dichloromethane and saturated aqueous sodium bicarbonate. The organic phase was concentrated and the resulting solid was triturated with methanol to afford 5-pyridazin-4-yl-1,2,4-thiadiazole as a beige solid.

¹H NMR (400 MHz, CDCl₃) 9.75 (dd, 1H) 9.45 (dd, 1H) 8.90 (s, 1H) 8.00 (dd, 1H)

### Example 6: Preparation of 2-[4-(1,2,4-oxadiazol-5-yl)pyridazin-1-ium-1-yl]ethyl sulfate A-8

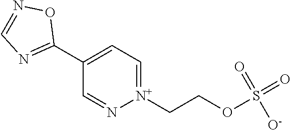 [SMILES: O=S(=O)([O-])OCC[n+]1ccc(-c2ncno2)cn1]

### Step 1: Preparation of 5-pyridazin-4-yl-1,2,4-oxadiazole

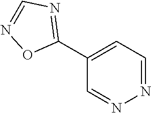 [SMILES: c1cc(-c2ncno2)cnn1]

To a mixture of N,N-(dimethylaminomethylene)pyridazine-4-carbothioamide (0.2 g), pyridine (0.17 mL) and ethanol (4 mL) at room temperature was added a solution of wet hydroxylamine-O-sulfonic acid (128 mg) in methanol (1.6 mL). After stirring overnight at room temperature the mixture was concentrated and partitioned between dichloromethane and saturated aqueous sodium bicarbonate. The organic layer was concentrated to afford 5-pyridazin-4-yl-1,2,4-oxadiazole.

¹H NMR (400 MHz, CDCl₃) 9.90 (dd, 1H) 9.52 (dd, 1H) 8.67 (s, 1H) 8.15 (dd, 1H)

### Step 2: Preparation of 2-[4-(1,2,4-oxadiazol-5-yl)pyridazin-1-ium-1-yl]ethyl sulfate A-8

A mixture of 5-pyridazin-4-yl-1,2,4-oxadiazole (0.056 g) and 1,3,2-dioxathiolane 2,2-dioxide (0.054 g) was heated in 1,2-dichloroethane (3 mL) at 85° C. overnight. The resulting precipitate was filtered off, washed with acetone and purified by preparative reverse phase HPLC to afford 2-[4-(1,2,4-oxadiazol-5-yl)pyridazin-1-ium-1-yl]ethyl sulfate as a 1:1 mixture with 2-[5-(1,2,4-oxadiazol-5-yl)pyridazin-1-ium-1-yl]ethyl sulfate.

1H NMR (400 MHz, DMSO-d₆) 10.32-10.28 (m, 1H) 10.18-10.12 (m, 1H) 9.55-9.54 (m, 1H) 9.43-9.38 (m, 1H) 5.26-5.14 (m, 2H) 4.43-4.33 (m, 2H)

The other isomer 2-[5-(1,2,4-oxadiazol-5-yl)pyridazin-1-ium-1-yl]ethyl sulfate has the structure below

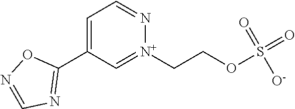 [SMILES: O=S(=O)([O-])OCC[n+]1cc(-c2ncno2)ccn1]

1H NMR (400 MHz, DMSO-d₆) 10.72 (s, 1H) 9.95-9.90 (m, 1H) 9.53-9.52 (m, 1H) 9.27-9.22 (m, 1H) 5.26-5.14 (m, 2H) 4.43-4.33 (m, 2H)

### Example 7: Preparation of 3-methyl-5-pyridazin-4-y-1,2,4-thiadiazole

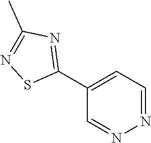

To a mixture of N,N-[1-(dimethylamino)ethylidene]pyridazine-4-carbothioamide (700 mg), pyridine (0.56 mL) and ethanol (18 mL) was added a solution of hydroxylamine-O-sulfonic acid (0.42 g) in methanol (7 mL) at room temperature. After one hour the reaction mixture was partitioned between dichloromethane and saturated aqueous sodium bicarbonate solution. The organic phase was concentrated and triturated with hexane to afford 3-methyl-5-pyridazin-4-yl-1,2,4-thiadiazole as a beige solid.

¹H NMR (400 MHz, CD₃OD) 9.76 (dd, 1H) 9.41 (dd, 1H) 8.25 (dd, 1H) 2.75 (s, 3H)

### Example 8: Preparation of 3-[4-(1-methylimidazol-2-yl)pyridazin-1-ium-1-yl]propanoic acid; 2,2,2-trifluoroacetate A-31

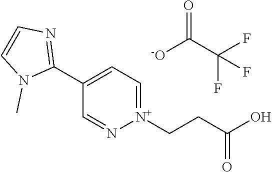

### Step 1: Preparation of ethyl 3-pyridazin-1-ium-1-ylpropanoate bromide

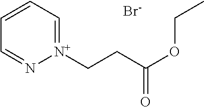

To a solution of pyridazine (1 g) in acetonitrile (40 mL) was added ethyl 3-bromopropanoate (1.76 mL) and the reaction was stirred at 80° C. for 25 hours. The mixture was concentrated and partitioned between dichloromethane and water. The aqueous layer was freeze dried to afford ethyl 3-pyridazin-1-ium-1-ylpropanoate bromide as a beige solid.

¹H NMR (400 MHz, D₂O) 9.68-9.92 (m, 1H) 9.43-9.56 (m, 1H) 8.43-8.69 (m, 2H) 5.15 (t, 2H) 4.11 (q, 2H) 3.27 (t, 2H) 1.16 (t, 3H)

### Step 2: Preparation of ethyl 3-[4-(1-methylimidazol-2-yl)-4H-pyridazin-1-yl]propanoate

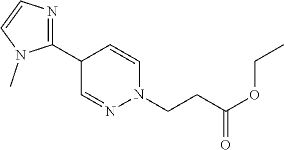 [SMILES: CCOC(=O)CCN1C=CC(c2nccn2C)C=N1]

To a solution of 1-methylimidazole (1 g) in tetrahydrofuran (10 mL) at −78° C. under a nitrogen atmosphere was added n-butyllithium (2.5M in hexanes, 5.4 mL) dropwise. After stirring at this temperature for 30 minutes zinc chloride (0.5M in THF, 7.7 mL) was added and the reaction mixture was allowed to warm to room temperature. To this mixture was added a solution of ethyl 3-pyridazin-1-ium-1-ylpropanoate (1.99 g) and iodocopper (2.1 g) in N,N-dimethylformamide (10 mL) and the reaction mixture was stirred at room temperature overnight. This reaction mixture was partitioned between ethyl acetate and water. The organic phase was concentrated to afford crude ethyl 3-[4-(1-methylimidazol-2-yl)-4H-pyridazin-1-yl]propanoate which was used directly in the next step. LC-MS 0.25 min MH+ 263.

### Step 3: Preparation of ethyl 3-[4-(1-methylimidazol-2-yl)pyridazin-1-ium-1-yl]propanoate

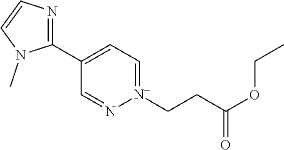 [SMILES: CCOC(=O)CC[n+]1ccc(-c2nccn2C)cn1]

To a solution of crude ethyl 3-[4-(1-methylimidazol-2-yl)-4H-pyridazin-1-yl]propanoate (2.52 g) in tetrahydrofuran (40 mL) was added 2,3,5,6-tetrachloro-1,4-benzoquinone (2.36 g) and the mixture stirred at room temperature for 2 hours. The reaction mixture was concentrated to afford crude ethyl 3-[4-(1-methylimidazol-2-yl)pyridazin-1-ium-1-yl]propanoate which was used directly in the next step.

LC-MS 0.26 min MH+ 261.

### Step 4: Preparation of 3-[4-(1-methylimidazol-2-yl)pyridazin-1-ium-1-yl]propanoic acid; 2,2,2-trifluoroacetate A-31

A mixture of crude ethyl 3-[4-(1-methylimidazol-2-yl)pyridazin-1-ium-1-yl]propanoate (320 mg) and 2M hydrochloric acid (6 mL) was heated at 80° C. for 2 hours. After cooling to room temperature the reaction mixture was concentrated and purified by preparative reverse phase HPLC to afford 3-[4-(1-methylimidazol-2-yl)pyridazin-1-ium-1-yl]propanoate as a purple gum.

¹H NMR (400 MHz, D₂O) 9.71-9.90 (m, 2H) 8.67-8.81 (m, 1H) 7.59-7.69 (m, 1H) 7.54 (d, 1H) 5.07 (t, 2H) 3.95-4.07 (m, 3H) 3.13-3.31 (m, 2H) (one CO₂H proton missing)

### Example 9: Preparation of 4-[1-methyl-5-(trifluoromethyl)pyrazol-3-yl]pyridazine (A) and 4-[2-methyl-5-(trifluoromethyl)pyrazol-3-yl]pyridazine (B)

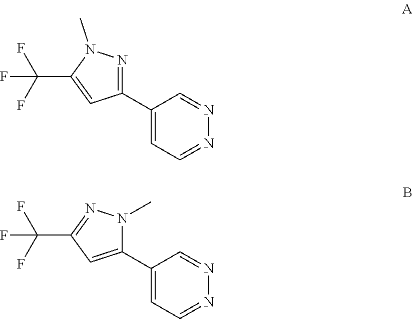 [SMILES: Cn1nc(-c2ccnnc2)cc1C(F)(F)F ; Cn1nc(C(F)(F)F)cc1-c1ccnnc1]

### Step 1: Preparation of 4,4,4-trifluoro-1-pyridazin-4-yl-butane-1,3-dione

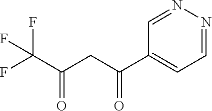 [SMILES: O=C(CC(=O)C(F)(F)F)c1ccnnc1]

To a mixture of ethyl 2,2,2-trifluoroacetate (0.256 g) and sodium methoxide (25% by wt in methanol, 0.449 mL) in tert-butyl methyl ether (0.409 mL) was added a suspension of 1-pyridazin-4-ylethanone (0.200 g) in tert-butyl methyl ether (2.87 mL) at room temperature and the mixture stirred at room temperature overnight. The reaction mixture was adjusted to pH 4 with 10% aqueous citric acid solution, diluted with water and extracted with dichloromethane (×3). Both liquid phases were concentrated, combined, then purified by preparative reverse phase HPLC to afford 4,4,4-trifluoro-1-pyridazin-4-yl-butane-1,3-dione as a brown gum. The product was a 2:1 mixture of the enol:keto tautomers.

¹H NMR (400 MHz, CD₃CN)

peaks for keto tautomer 9.57 (s, 1H) 9.51-9.43 (m, 1H) 8.04-7.98 (m, 1H) 3.52 (s, 2H)

peaks for enol tautomer (shown below) 9.64 (s, 1H) 9.5-9.44 (m, 1H) 8.10-8.04 (m, 1H) 6.96 (s, 1H)

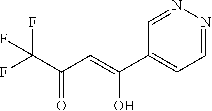 [SMILES: O=C(/C=C(\O)c1ccnnc1)C(F)(F)F]

### Step 2: Preparation of 4-[1-methyl-5-(trifluoromethyl)pyrazol-3-yl]pyridazine (A) and 4-[2-methyl-5-(trifluoromethyl)pyrazol-3-yl]pyridazine (B)

Methylhydrazine (0.54 mL) was added slowly to a solution of 4,4,4-trifluoro-1-pyridazin-4-yl-butane-1,3-dione (1.5 g) in ethanol (11 mL), followed by heating at reflux for 4 hours.

After cooling to room temperature the mixture was concentrated and the residue dissolved in tetrahydrofuran (34 mL). To this solution was added 3M aqueous hydrochloric acid (6.9 mL), followed by heating at reflux for 2 hours. The reaction mixture was cooled to room temperature and allowed to stand overnight. The mixture was concentrated and purified by preparative reverse phase HPLC to afford 4-[1-methyl-5-(trifluoromethyl)pyrazol-3-yl]pyridazine (A) as an orange solid and 4-[2-methyl-5-(trifluoromethyl)pyrazol-3-yl]pyridazine (B) as an orange liquid.

4-[1-methyl-5-(trifluoromethyl)pyrazol-3-yl]pyridazine (A) 1H NMR (400 MHz, CD₃CN) 9.70-9.65 (m, 1H) 9.31-9.27 (m, 1H) 8.11 (dd, 1H) 7.45 (s, 1H) 4.11 (s, 3H)

4-[2-methyl-5-(trifluoromethyl)pyrazol-3-yl]pyridazine (B) 1H NMR (400 MHz, CD₃CN) 9.43-9.39 (m, 1H) 9.38-9.35 (m, 1H) 7.87 (dd, 1H) 7.05 (s, 1H) 4.04 (s, 3H)

### Example 10: Preparation of 4-(2-methyltetrazol-5-yl)pyridazine

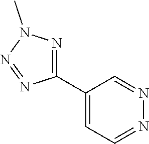

### Step 1: Preparation of 4-(1H-tetrazol-5-yl)pyridazine

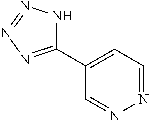 [SMILES: c1cc(-c2nnn[nH]2)cnn1]

To a mixture of pyridazine-4-carbonitrile (0.200 g), sodium azide (0.187 g) and copper sulfate pentahydrate (0.048 g) was added dimethyl sulfoxide (0.4 mL). This mixture was heated at 145° C. under microwave irradiation for 10 minutes. After cooling to room temperature the reaction mixture was quenched with ice cold water (20 mL), acidified with 1M aqueous hydrochloric acid and extracted with a 9:1 ratio of ethyl acetate and methanol (3×30 mL). The combined organic phases were concentrated to afford crude 4-(1H-tetrazol-5-yl)pyridazine.

¹H NMR (400 MHz, DMSO-d₆) 9.67 (br s, 1H) 9.15 (br d, 1H) 8.00 (br d, 1H) (one NH proton missing)

### Step 2: Preparation of 4-(2-methyltetrazol-5-yl)pyridazine

A mixture of 4-(1H-tetrazol-5-yl)pyridazine (0.16 g), N,N-dimethylformamide (1 mL), dimethyl carbonate (0.5 mL) and 1,4-diazabicyclo[2.2.2]octane (0.026 g) was heated at 150° C. under microwave irradiation for 80 minutes. After cooling to room temperature the reaction mixture was concentrated and purified by silica gel chromatography eluting with 0 to 10% methanol in dichloromethane to afford 4-(2-methyltetrazol-5-yl)pyridazine as a brown solid.

¹H NMR (400 MHz, CD₃OD) 9.85 (s, 1H) 9.39 (d, 1H) 8.34 (d, 1H) 4.50 (s, 3H)

The other isomer, 4-(1-methyltetrazol-5-yl)pyridazine was also obtained from this reaction.

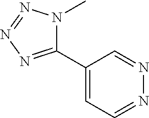

¹H NMR (400 MHz, CD₃OD) 9.71 (s, 1H) 9.48 (d, 1H) 8.23 (d, 1H) 4.34 (s, 3H)

### Example 11: Preparation of 4-methyl-2-pyridazin-4-yl-thiazole

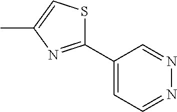

To a solution of pyridazine-4-carbothioamide (0.5 g) in ethanol (10 mL) was added 1-chloropropan-2-one (0.432 g), followed by heating at 80° C. for 5 hours. The reaction mixture was concentrated and the residue dissolved in distilled water. The aqueous phase was basified with saturated aqueous sodium bicarbonate and extracted with dichloromethane. The organic phase was concentrated and purified by silica gel chromatography eluting with 60 to 80% ethyl acetate in cyclohexane to afford 4-methyl-2-pyridazin-4-yl-thiazole.

¹H NMR (400 MHz, CD₃OD) 9.70-9.80 (m, 1H) 9.24-9.33 (m, 1H) 8.08-8.24 (m, 1H) 7.47 (s, 1H) 2.55 (s, 3H)

### Example 12: Preparation of 4,5-dimethyl-2-pyridazin-4-yl-oxazole

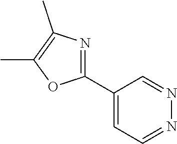

### Step 1: Preparation of N-(1-methylprop-2-ynyl)pyridazine-4-carboxamide

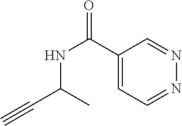

To a mixture of methyl pyridazine-4-carboxylate (1.00 g) in methanol (4 mL) was added 1-methylprop-2-ynylammonium chloride (2.29 g) and N,N-diisopropylethylamine (3.92 mL), followed by heating at 100° C. under microwave irradiation for 2 hours. After cooling to room temperature the reaction mixture was concentrated and purified by silica gel chromatography eluting with ethyl acetate to afford N-(1-methylprop-2-ynyl)pyridazine-4-carboxamide.

¹H NMR (400 MHz, CDCl₃) 9.49-9.67 (m, 1H) 9.38 (dd, 1H) 7.87 (dd, 1H) 6.83-7.07 (m, 1H) 4.94-5.13 (m, 1H) 2.37 (d, 1H) 1.48-1.63 (m, 3H)

### Step 2: Preparation of N-(2-bromo-1-methyl-allyl)pyridazine-4-carboxamide

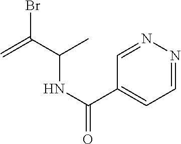

A mixture of N-(1-methylprop-2-ynyl)pyridazine-4-carboxamide (0.27 g) and hydrobromic acid (5.4 mL, 33% wt in acetic acid) was heated at 60° C. for 18 hours. After cooling to room temperature, saturated aqueous sodium bicarbonate was added and the product was extracted with ethyl acetate. The organic phase was concentrated to afford crude N-(2-bromo-1-methyl-allyl)pyridazine-4-carboxamide which was used directly in the next step.

### Step 3: Preparation of 4,5-dimethyl-2-pyridazin-4-yl-oxazole

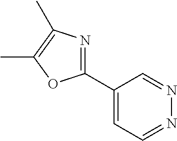

To a solution of crude N-(2-bromo-1-methyl-allyl)pyridazine-4-carboxamide (0.25 g) in dimethyl sulfoxide (2.5 mL) under nitrogen atmosphere was added cesium carbonate (1.05 g), followed by heating at 110° C. for 1 hour. After cooling to room temperature, aqueous saturated lithium chloride was added and the crude product was extracted with ethyl acetate. The organic phase was dried over sodium sulfate, concentrated and purified by silica gel chromatography eluting with ethyl acetate to afford 4,5-dimethyl-2-pyridazin-4-yl-oxazole.

¹H NMR (400 MHz, CDCl₃) 9.63-9.90 (m, 1H) 9.23-9.47 (m, 1H) 7.92-8.15 (m, 1H) 2.34-2.51 (m, 3H) 2.22 (m, 3H)

### Example 13: Preparation of 3-[4-(5-methyloxazol-2-yl)pyridazin-1-ium-1-yl]propanoic acid; 2,2,2-trifluoroacetate A-15

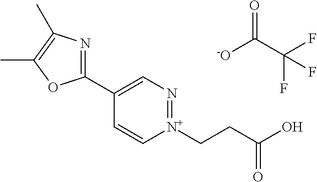

### Step 1: Preparation of N-prop-2-ynylpyridazine-4-carboxamide

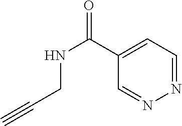

To a solution of methyl pyridazine-4-carboxylate (1 g) in methanol (2.5 mL) was added prop-2-yn-1-amine (4 g) and the mixture was heated at 100° C. under microwave irradiation for 4 hours. After cooling to room temperature, the reaction mixture was concentrated and purified by silica gel chromatography eluting with 90-100% ethyl acetate in cyclohexane to afford N-prop-2-ynylpyridazine-4-carboxamide as white solid.

¹H NMR (400 MHz, DMSO-d₆) 9.53-9.56 (m, 1H) 9.47-9.52 (m, 1H) 9.42-9.46 (m, 1H) 7.96-8.03 (m, 1H) 4.06-4.16 (m, 2H) 3.19-3.26 (m, 1H)

### Step 2: Preparation of N-(2-bromoallyl)pyridazine-4-carboxamide

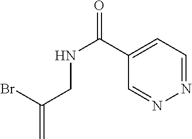

A mixture of N-prop-2-ynylpyridazine-4-carboxamide (0.5 g) and hydrobromic acid (10 mL, 33% wt in acetic acid) was heated at 60° C. for 18 hours. After cooling to room temperature, water was added and the mixture was basified with aqueous saturated sodium bicarbonate. This aqueous mixture was extracted with ethyl acetate and the organic phase was further washed with brine, dried over sodium sulfate and concentrated to afford crude N-(2-bromoallyl)pyridazine-4-carboxamide which was used directly in the next step.

### Step 3: Preparation of 5-methyl-2-pyridazin-4-yl-oxazole

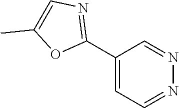

To a mixture of crude N-(2-bromoallyl)pyridazine-4-carboxamide (0.1 g) in dimethyl sulfoxide (1 mL) under nitrogen atmosphere was added cesium carbonate (0.222 g) and the mixture heated at 110° C. for 3 hours. The reaction mixture was cooled to room temperature, diluted with water (40 mL) and extracted with ethyl acetate (3×20 mL). The combined organic phases were washed with saturated aqueous lithium chloride (30 mL), brine (30 mL) and dried over anhydrous sodium sulfate. Concentration of the organic filtrate afforded 5-methyl-2-pyridazin-4-yl-oxazole.

¹H NMR (400 MHz, CDCl₃) 9.78 (s, 1H) 9.36 (d, 1H) 8.06 (dd, 1H) 7.05 (s, 1H) 2.49 (s, 3H)

### Step 4: Preparation of ethyl 3-[4-(5-methyloxazol-2-yl)pyridazin-1-ium-1-yl]propanoate bromide

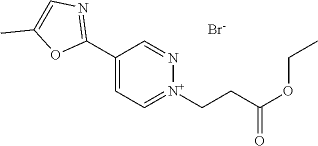 [SMILES: CCOC(=O)CC[n+]1ccc(-c2ncc(C)o2)cn1 ; [Br-]]

To a solution of 5-methyl-2-pyridazin-4-yl-oxazole (0.1 g) in acetonitrile (2 mL) was added ethyl 3-bromopropanoate (0.159 mL), and the mixture was heated at 80° C. for 18 hours. After cooling to room temperature the solution was concentrated and the residue triturated with tert-butylmethyl ether to afford a crude 1:1 mixture of ethyl 3-[4-(5-methyloxazol-2-yl)pyridazin-1-ium-1-yl]propanoate bromide and ethyl 3-[5-(5-methyloxazol-2-yl)pyridazin-1-ium-1-yl]propanoate bromide which was used directly in the next step.

### Step 5: Preparation of 3-[4-(5-methyloxazol-2-yl)pyridazin-1-ium-1-yl]propanoic acid 2,2,2-trifluoroacetate A-15

A crude 1:1 mixture of ethyl 3-[4-(5-methyloxazol-2-yl)pyridazin-1-ium-1-yl]propanoate bromide and ethyl 3-[5-(5-methyloxazol-2-yl)pyridazin-1-ium-1-yl]propanoate bromide (0.2 g) in 2M hydrochloric acid (4 mL) was stirred at room temperature for 18 hours. The reaction mixture was concentrated and purified by preparative reverse phase HPLC to afford 3-[4-(5-methyloxazol-2-yl)pyridazin-1-ium-1-yl]propanoic acid 2,2,2-trifluoroacetate.

¹H NMR (400 MHz, D₂O) 9.82 (d, 1H) 9.74 (d, 1H) 8.77 (dd, 1H) 7.29 (d, 1H) 5.06 (t, 2H) 3.23 (t, 2H) 2.47 (d, 3H) (one CO₂H proton missing)

### Example 14: Preparation of 4-(2-methyl-1,2,4-triazol-3-yl)pyridazine

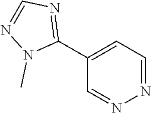

### Step 1: Preparation of N-(dimethylaminomethylene)pyridazine-4-carboxamide

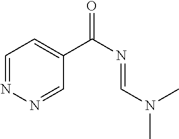

A mixture of pyridazine-4-carboxamide (2 g) and 1,1-dimethoxy-N,N-dimethyl-methanamine (20 mL), under nitrogen atmosphere, was heated at reflux for 1 hour. The reaction mixture was concentrated and the residue washed with cyclohexane (3×20 mL) to afford N-(dimethylaminomethylene) pyridazine-4-carboxamide which was used directly in the next step.

### Step 2: Preparation of 4-(2-methyl-1,2,4-triazol-3-yl)pyridazine

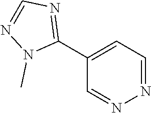

To a mixture of N-(dimethylaminomethylene)pyridazine-4-carboxamide (0.5 g), acetic acid (5 mL) and 1,4-dioxane (5 mL) was added methylhydrazine sulfate (0.404 g). This mixture was heated at 70° C. under microwave irradiation for 30 minutes. After cooling to room temperature, the reaction mixture was concentrated and extracted with ethyl acetate (3×100 mL). The organic layers were concentrated and purified by silica gel chromatography eluting with 0 to 90% methanol in dichloromethane to afford 4-(2-methyl-1,2,4-triazol-3-yl)pyridazine.

¹H NMR (400 MHz, CDCl₃) 9.62 (dd, 1H) 9.41 (dd, 1H) 8.04 (s, 1H) 7.86 (dd, 1H) 4.15 (s, 3H)

### Example 15: Preparation of 2-pyridazin-4-y-4-(trifluoromethyl)thiazole

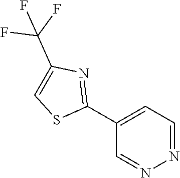 [SMILES: FC(F)(F)c1csc(-c2ccnnc2)n1]

To a mixture of pyridazine-4-carbothioamide (0.05 g) and ethanol (0.25 mL) was added 3-bromo-1,1,1-trifluoro-propan-2-one (0.089 g). The resulting mixture was heated at reflux for 6 hours then left to stand overnight. The reaction mixture was concentrated and dissolved in water (50 mL). The aqueous phase was adjusted to pH 7-8 with saturated aqueous sodium bicarbonate solution and extracted with ethyl acetate (3×80 mL). The organic layers were concentrated and purified by silica gel chromatography eluting with 50-60% ethyl acetate in cyclohexane to afford 2-pyridazin-4-yl-4-(trifluoromethyl)thiazole.

¹H NMR (400 MHz, CD₃CN) 9.73 (dd, 1H) 9.37 (dd, 1H) 8.32 (d, 1H) 8.06 (dd, 1H)

### Example 16 Preparation of 4-chloro-2-pyridazin-4-yl-thiazole

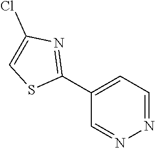 [SMILES: Clc1csc(-c2ccnnc2)n1]

### Step 1: Preparation of tributyl(pyridazin-4-yl)stannane

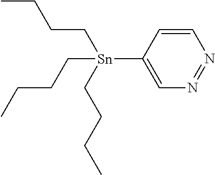 [SMILES: CCC[CH2][Sn]([CH2]CCC)([CH2]CCC)[c]1ccnnc1]

To a solution of lithium diisopropylamide (1M solution in tetrahydrofuran, 125 mL) at −78° C. under nitrogen was added a solution of pyridazine (10 g) and tri-n-butyltin chloride (44.6 g) in THF (100 mL) drop wise. The reaction mixture was stirred at −78° C. for 1 hour. The reaction mixture was warmed to room temperature and quenched with saturated aqueous ammonium chloride (100 mL) and extracted with ethyl acetate (3×150 mL). The organic layer was dried over sodium sulfate, concentrated and purified by chromatography on silica eluting with 30% ethyl acetate in hexanes to afford tributyl(pyridazin-4-yl)stannane as a pale brown liquid.

¹H NMR (400 MHz, CDCl₃) 9.17 (t, 1H) 9.02 (dd, 1H) 7.54 (dd, 1H) 1.57-1.49 (m, 6H) 1.37-1.29 (m, 6H) 1.19-1.13 (m, 6H) 0.92-0.86 (m, 9H).

### Step 2: Preparation of 4-chloro-2-pyridazin-4-yl-thiazole

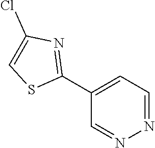 [SMILES: Clc1csc(-c2ccnnc2)n1]

To a solution of 2,4-dichlorothiazole (1 g) in 1,4-dioxane (15 mL) was added tributyl(pyridazin-4-yl)stannane (2.876 g), tetrakis(triphenylphosphine) palladium(0) (0.376 g), cuprous iodide (0.371 g) and lithium chloride (0.826 g). The reaction mixture was purged with nitrogen then heated at 130° C. under microwave irradiation for 40 minutes. After cooling to room temperature the mixture was filtered through Celite and washed with methanol. The filtrate was concentrated and purified by silica gel chromatography eluting with 50-60% ethyl acetate in cyclohexane to afford 4-chloro-2-pyridazin-4-yl-thiazole.

¹H NMR (400 MHz, DMSO-d₆) 9.60-9.88 (m, 1H) 9.40 (d, 1H) 8.15 (dd, 1H) 8.11 (s, 1H)

### Example 17: Preparation of 4-methoxy-2-pyridazin-4-yl-thiazole

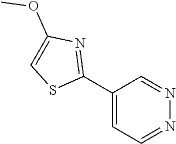

A mixture of 4-chloro-2-pyridazin-4-yl-thiazole (0.2 g) in sodium methoxide (30% in methanol, 5 mL) was heated at 100° C. under microwave irradiation for 60 minutes. The reaction mixture was concentrated and purified by silica gel chromatography eluting with 50-60% ethyl acetate in cyclohexane to afford 4-methoxy-2-pyridazin-4-yl-thiazole.

¹H NMR (400 MHz, CDCl₃) 9.64-9.75 (m, 1H) 9.28 (d, 1H) 7.90 (dd, 1H) 6.39 (s, 1H) 4.03 (s, 3H)

### Example 18: Preparation of N-methyl-5-pyridazin-4-y-1,3,4-thiadiazol-2-amine

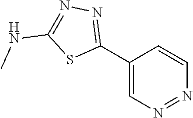

### Step 1: Preparation of 1-methyl-3-(pyridazine-4-carbonylamino)thiourea

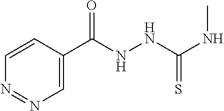

To a mixture of pyridazine-4-carbohydrazide (2 g) and propan-2-ol (40 mL) was added methyl isothiocyanate (1.059 g) and the mixture heated at reflux for 3 hours. After cooling to 0° C. the resulting precipitate was filtered, washed with tert-butylmethyl ether (2×50 mL) and dichloromethane (10 mL) and dried to afford 1-methyl-3-(pyridazine-4-carbonylamino)thiourea as a yellow solid.

¹H NMR (400 MHz, DMSO-d₆) 10.89 (br s, 1H) 9.56-9.62 (m, 1H) 9.46-9.56 (m, 2H) 8.20 (br d, 1H) 8.04 (dd, 1H) 2.88 (d, 3H)

### Step 2: Preparation of N-methyl-5-pyridazin-4-yl-1,3,4-thiadiazol-2-amine

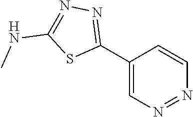

A mixture of 1-methyl-3-(pyridazine-4-carbonylamino)thiourea (0.4 g) and concentrated sulfuric acid (4 mL) was stirred at room temperature for 12 hours. The reaction mixture was cooled over ice and carefully basified with aqueous ammonium hydroxide (28-30% NH₃). The resulting precipitate was filtered off, washed with water then dried to afford N-methyl-5-pyridazin-4-yl-1,3,4-thiadiazol-2-amine.

¹H NMR (400 MHz, DMSO-d₆) 9.60 (dd, 1H) 9.29 (dd, 1H) 8.33 (br s, 1H) 7.93 (dd, 1H) 2.98 ppm (s, 3H)

### Example 19: Preparation of 5-pyridazin-4-y-1,3,4-thiadiazol-2-amine

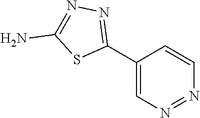 [SMILES: Nc1nnc(-c2ccnnc2)s1]

### Step 1: Preparation of (2,3,4,5,6-pentafluorophenyl) pyridazine-4-carboxylate

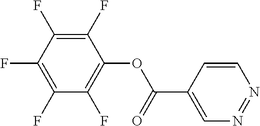 [SMILES: O=C(Oc1c(F)c(F)c(F)c(F)c1F)c1ccnnc1]

To a solution of pyridazine-4-carboxylic acid (5 g) in dichloromethane (50 mL) under a nitrogen atmosphere was added 2,3,4,5,6-pentafluorophenol (7.194 g). The mixture was cooled to 0° C. and 3-(ethyliminomethyleneamino)-N,N-dimethyl-propan-1-amine hydrochloride (8.99 g) and N,N-dimethylaminopyridine (0.964 g) were added. After 4 hours the reaction mixture was diluted with water and extracted with ethyl acetate. The organic phase was washed with water, brine and dried over sodium sulfate. The organic layer was concentrated and purified by silica gel chromatography eluting with 35% ethyl acetate in hexane to afford (2,3,4,5,6-pentafluorophenyl) pyridazine-4-carboxylate as a white solid.

¹H NMR (400 MHz, CDCl₃) 9.90-9.76 (m, 1H) 9.67-9.47 (m, 1H) 8.36-7.88 (m, 1H)

### Step 2: Preparation of (pyridazine-4-carbonylamino)thiourea

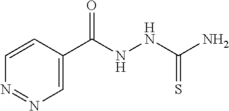 [SMILES: NC(=S)NNC(=O)c1ccnnc1]

To a solution of (2,3,4,5,6-pentafluorophenyl) pyridazine-4-carboxylate (1 g) in acetonitrile (20 mL) was added thiosemicarbazide (0.377 g), followed by heating at 70° C. for 12 hours. After cooling to room temperature the reaction mixture was concentrated and purified by silica gel chromatography eluting with 0 to 10% methanol in dichloromethane to afford (pyridazine-4-carbonylamino)thiourea.

¹H NMR (400 MHz, DMSO-d₆) 10.93 (br s, 1H) 9.50-9.59 (m, 2H) 9.46 (d, 1H) 8.02 (dd, 2H) 7.83 (br s, 1H)

### Step 3: Preparation of 5-pyridazin-4-yl-1,3,4-thiadiazol-2-amine

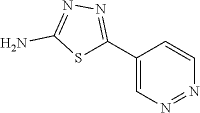 [SMILES: Nc1nnc(-c2ccnnc2)s1]

A mixture of (pyridazine-4-carbonylamino)thiourea (1 g) and concentrated sulfuric acid (10 mL) was stirred at room temperature for 12 hours. The reaction mixture was the cooled over ice and carefully basified with aqueous ammonium hydroxide (28-30% NH₃). The resulting precipitate was filtered off, washed with water and dried to afford 5-pyridazin-4-yl-1,3,4-thiadiazol-2-amine as a light green solid.

¹H NMR (400 MHz, DMSO-d₆) 9.57-9.67 (m, 1H) 9.19-9.34 (m, 1H) 7.91-7.98 (m, 1H) 7.78-7.90 (m, 2H)

### Example 20: Preparation of 4-(4-methyl-1,2,4-triazol-3-yl)pyridazine

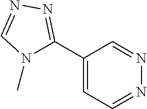

### Step 1: Preparation of 4-methyl-3-pyridazin-4-yl-1H-1,2,4-triazole-5-thione

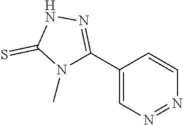

A mixture of 1-methyl-3-(pyridazine-4-carbonylamino)thiourea (0.750 g) and acetic acid (10 mL) was heated at 120° C. for 16 hours. The reaction mixture was concentrated and the crude solid was triturated with tert-butylmethyl ether (30 mL) to afford 4-methyl-3-pyridazin-4-yl-1H-1,2,4-triazole-5-thione.

¹H NMR (400 MHz, DMF-d₇) 14.73 (br s, 1H) 10.00 (dd, 1H) 9.89 (dd, 1H) 8.53 (dd, 1H) 4.07 (s, 3H)

### Step 2: Preparation of 4-(4-methyl-1,2,4-triazol-3-yl)pyridazine

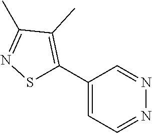

To a mixture of Raney nickel (1.25 g, washed with ethanol, weighed approximately) in ethanol (8 mL), under nitrogen atmosphere, was added 4-methyl-3-pyridazin-4-yl-1H-1,2,4-triazole-5-thione (0.4 g) and the mixture heated at 90° C. for 20 hours. The reaction mixture was filtered through Celite, washed with ethanol and the filtrate was concentrated to afford 4-(4-methyl-1,2,4-triazol-3-yl)pyridazine.

¹H NMR (400 MHz, CDCl₃) 9.60 (br s, 1H) 9.34 (br s, 1H) 8.23-8.40 (m, 1H) 7.86 (br s, 1H) 3.90 (br s, 3H)

### Example 21: Preparation of 2-[4-(3,4-dimethylisothiazol-5-yl)pyridazin-1-ium-1-yl]ethanesulfonate A-54

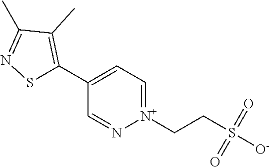 [SMILES: Cc1nsc(-c2cc[n+](CCS(=O)(=O)[O-])nc2)c1C]

To a mixture of 4,5-dibromo-3-methyl-isothiazole (1.95 g) in degassed 1,4-dioxane (29.3 mL), under nitrogen atmosphere, was added tetrakis(triphenylphosphine)palladium(0) (1.12 g) and tributyl(pyridazin-4-yl)stannane (2.02 g) and the reaction mixture was heated at 100° C. for 18 hours. After cooling to room temperature, potassium carbonate (1.82 g) and methylboronic acid (0.985 g) were added and the reaction mixture was heated at 100° C. for a further 20 hours. After cooling to room temperature, the reaction mixture was filtered through Celite and washed through with methanol. The filtrate was concentrated and purified by silica gel chromatography eluting with 0 to 10% methanol in dichloromethane to afford 3,4-dimethyl-5-pyridazin-4-yl-isothiazole which was used directly in the next step.

### Step 2: Preparation of 2-[4-(3,4-dimethylisothiazol-5-yl)pyridazin-1-ium-1-yl]ethanesulfonate A-54

A mixture of crude 3,4-dimethyl-5-pyridazin-4-yl-isothiazole (0.3 g) and sodium 2-bromoethanesulfonate (0.397 g) in water (6 mL) and 1,4-dioxane (6 mL) was heated at reflux for 48 hours. The reaction mixture was concentrated, washed with ethyl acetate and purified by preparative reverse phase HPLC to afford 2-[4-(3,4-dimethylisothiazol-5-yl)pyridazin-1-ium-1-yl]ethanesulfonate.

¹H NMR (400 MHz, D₂O) 9.73 (dd, 1H) 9.59 (dd, 1H) 8.61 (dd, 1H) 5.20 (t, 2H) 3.67 (t, 2H) 2.44 (s, 3H) 2.35 (s, 3H)

### Example 22: Preparation of 3-pyridazin-4-ylisoxazole

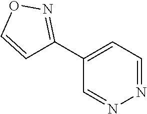 [SMILES: c1cc(-c2ccon2)cnn1]

### Step 1: Preparation of pyridazine-4-carbaldehyde Oxime

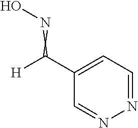

To a solution of pyridazine-4-carbaldehyde (2 g) in ethanol (29.6 mL) was added a solution of sodium acetate (1.53 g) and hydroxylamine hydrochloride (1.29 g) in distilled water (69.4 mL). This resulting mixture was heated at reflux for 2 hours. The reaction mixture was cooled to room temperature, concentrated and the resulting orange residue was triturated with water and azeotroped with methanol to afford pyridazine-4-carbaldehyde oxime as a beige solid.

¹H NMR (400 MHz, CD₃OD) 9.41-9.37 (m, 1H) 9.17 (dd, 1H) 8.14 (s, 1H) 7.82 (dd, 1H) (one OH proton missing)

### Step 2: Preparation of trimethyl-(3-pyridazin-4-ylisoxazol-5-yl)silane

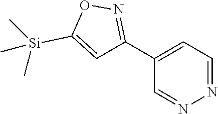

To a solution of pyridazine-4-carbaldehyde oxime (1.45 g) in acetonitrile (141 mL) at 50° C. was added a solution of N-chlorosuccinimide (3.81 g) in acetonitrile (23.6 mL) and this mixture was heated at this temperature for 1 hour. Ethynyl(trimethyl)silane (17 mL) was added to the reaction mixture, followed by triethylamine (1.81 mL) and heating was continued for a further 3.5 hours. The reaction mixture was cooled to room temperature, concentrated and purified by silica gel chromatography eluting with 0 to 100% ethyl acetate in iso-hexane to afford trimethyl-(3-pyridazin-4-ylisoxazol-5-yl)silane as a yellow solid.

¹H NMR (400 MHz, CDCl₃) 9.64-9.59 (m, 1H) 9.32 (dd, 1H) 7.88 (dd, 1H) 6.86 (s, 1H) 0.42 (s, 9H)

### Step 3: Preparation of 3-pyridazin-4-ylisoxazole

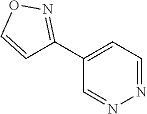 [SMILES: c1cc(-c2ccon2)cnn1]

To a solution of trimethyl-(3-pyridazin-4-ylisoxazol-5-yl)silane (0.100 g) in ethanol (2.51 mL) was added aqueous ammonium hydroxide (28-30% NH₃, 0.150 mL) and the reaction mixture was stirred at room temperature for 20 hours. The reaction mixture was concentrated to afford 3-pyridazin-4-ylisoxazole as a brown gum.

¹H NMR (400 MHz, CD₃OD) 9.74-9.67 (m, 1H), 9.35 (dd, 1H), 8.91 (d, 1H), 8.17 (dd, 1H), 7.19 (d, 1H)

### Example 23: Preparation of 2-(4-oxazol-4-ylpyridazin-1-ium-1-yl)ethanesulfonate A-22

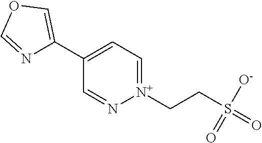 [SMILES: O=S(=O)([O-])CC[n+]1ccc(-c2cocn2)cn1]

### Step 1: Preparation of 2,2-dimethylpropyl 2-(2-tert-butoxycarbonylhydrazino)ethanesulfonate

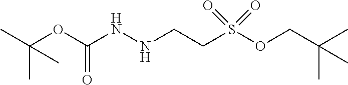 [SMILES: CC(C)(C)COS(=O)(=O)CCNNC(=O)OC(C)(C)C]

To a solution of 2,2-dimethylpropyl ethenesulfonate (1.35 g) in methanol (10.1 mL) was added tert-butyl carbazate (1 g) and the mixture heated at 70° C. for 24 hours. The reaction mixture was then concentrated to afford 2,2-dimethylpropyl 2-(2-tert-butoxycarbonylhydrazino)ethanesulfonate as a yellow liquid.

¹H NMR (400 MHz, CDCl₃) 3.90 (s, 2H) 3.38-3.30 (m, 4H) 1.50-1.43 (s, 9H) 1.00-0.97 (s, 9H) (two NH proton missing)

### Step 2: Preparation of [2-(2,2-dimethylpropoxysulfonyl)ethylamino]ammonium Chloride

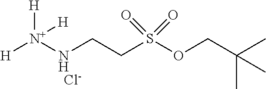

A solution of 2,2-dimethylpropyl 2-(2-tert-butoxycarbonylhydrazino)ethanesulfonate (1 g) in 3M methanolic hydrogen chloride (24.2 mL) was heated at 70° C. for 7 hours. After cooling to room temperature, the reaction mixture was concentrated to afford [2-(2,2-dimethylpropoxysulfonyl)ethylamino]ammonium chloride as a pink gum that solidified on standing.

¹H NMR (400 MHz, CD₃OD) 3.95 (s, 2H) 3.59-3.53 (m, 2H) 3.44-3.39 (m, 2H) 1.00 (s, 9H) (three NH proton missing)

### Step 3: Preparation of 2,2-dimethylpropyl 2-pyridazin-1-ium-1-ylethanesulfonate chloride

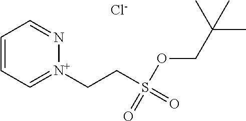 [SMILES: CC(C)(C)COS(=O)(=O)CC[n+]1ccccn1 ; [Cl-]]

A solution of 2,5-dimethoxy-2,5-dihydrofuran (0.5 g) in acetic acid (2 mL) and water (1 mL) was stirred at room temperature for 4 hours. To this was added a solution of [2-(2,2-dimethylpropoxysulfonyl)ethylamino]ammonium chloride (1.04 g) in water (1 mL) and the mixture was stirred at room temperature overnight. The reaction mixture was concentrated and purified by silica gel chromatography eluting with 0 to 50% methanol in dichloromethane to afford 2,2-dimethylpropyl 2-pyridazin-1-ium-1-ylethanesulfonate chloride.

¹H NMR (400 MHz, CDCl₃) 11.11 (d, 1H) 9.45 (d, 1H) 8.96 (ddd, 1H) 8.58 (dd, 1H) 5.68 (t, 2H) 4.30 (t, 2H) 3.99 (s, 2H) 0.98 (s, 9H)

### Step 4: Preparation of 2,2-dimethylpropyl 2-(4-oxazol-4-yl-4H-pyridazin-1-yl)ethanesulfonate

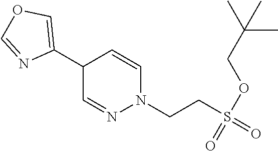 [SMILES: CC(C)(C)COS(=O)(=O)CCN1C=CC(c2cocn2)C=N1]

A solution of oxazole (1 g) was stirred in tetrahydrofuran (10 mL) and cooled to −78° C. under nitrogen atmosphere. A solution of n-butyllithium (2.5M in hexanes, 5.8 mL) was added dropwise and the reaction mixture was stirred for 30 minutes. Zinc chloride (0.5M in THF, 9 mL) was added and the reaction mixture was allowed to warm to room temperature. To this was added a solution of 2,2-dimethylpropyl 2-pyridazin-1-ium-1-ylethanesulfonate (3.4 g) and iodocopper (2.5 g) in N,N-dimethylformamide (10 mL) and the reaction mixture was stirred at room temperature overnight. The reaction mixture was partitioned between EtOAc and water and the organic layer was concentrated to give 2,2-dimethylpropyl 2-(4-oxazol-4-yl-4H-pyridazin-1-yl)ethanesulfonate as a dark green gum. This material was used without further purification in the subsequent step.

### Step 5: Preparation of 2,2-dimethylpropyl 2-(4-oxazol-4-ylpyridazin-1-ium-1-yl)ethanesulfonate; 2,2,2-trifluoroacetate A-21

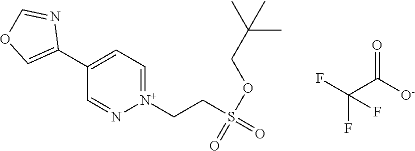 [SMILES: CC(C)(C)COS(=O)(=O)CC[n+]1ccc(-c2cocn2)cn1 ; O=C([O-])C(F)(F)F]

To a solution of crude 2,2-dimethylpropyl 2-(4-oxazol-4-yl-4H-pyridazin-1-yl)ethanesulfonate (5.32 g) in tetrahydrofuran (160 mL) was added 2,3,5,6-tetrachloro-1,4-benzoquinone (4 g) and the reaction mixture was stirred for 2 hours at room temperature. The reaction mixture was concentrated, washed with ethyl acetate and purified by preparative reverse phase HPLC to give 2,2-dimethylpropyl 2-(4-oxazol-4-ylpyridazin-1-ium-1-yl)ethanesulfonate; 2,2,2-trifluoroacetate as a brown gum.

¹H NMR (400 MHz, DMSO-d₆) 10.04 (d, 1H) 9.97 (d, 1H) 9.45 (s, 1H) 8.99-8.94 (m, 1H) 8.89 (d, 1H) 5.24 (s, 2H) 4.26 (t, 2H) 3.95 (s, 2H) 0.91 (s, 9H)

### Step 6: Preparation of 2-(4-oxazol-4-ylpyridazin-1-ium-1-yl)ethanesulfonate A-22

A mixture of 2,2-dimethylpropyl 2-(4-oxazol-2-ylpyridazin-1-ium-1-yl)ethanesulfonate 2,2,2-trifluoroacetate (0.2 g), trifluoroacetic acid (3 mL) and anisole (0.6 mL) was heated at 80° C. for 1.5 hours. The reaction mixture was freeze dried and purified by preparative reverse phase HPLC to give 2-(4-oxazol-4-ylpyridazin-1-ium-1-yl)ethanesulfonate as a pale yellow solid.

¹H NMR (400 MHz, D₂O) 9.70-9.77 (m, 1H) 9.58 (d, 1H) 8.88-8.97 (m, 1H) 8.63-8.70 (m, 1H) 8.35-8.42 (m, 1H) 5.04-5.18 (m, 2H) 3.53-3.71 (m, 2H)

### Example 24: Preparation of 3-[4-(thiadiazol-4-yl)pyridazin-1-ium-1-yl]propanoic acid 2,2,2-trifluoroacetate A139

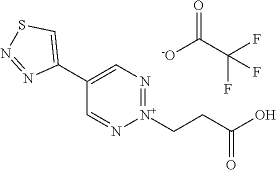

### Step 1: Preparation of tert-butyl N-[(E)-1-pyridazin-4-ylethylideneamino]carbamate

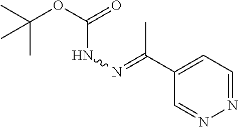 [SMILES: CC(=NNC(=O)OC(C)(C)C)c1ccnnc1]

To a solution of 1-pyridazin-4-ylethanone (0.3 g) in 1,4-dioxane (1.5 mL) was added tert-butyl N-aminocarbamate (0.327 g) and the reaction heated at 70° C. for 90 minutes. The reaction mixture was concentrated to give tert-butyl N-[(E)-1-pyridazin-4-ylethylideneamino]carbamate which was used without further purification.

¹H NMR (400 MHz, CDCl₃) 9.59 (dd, 1H), 9.19 (dd, 1H), 8.00 (s, 1H), 7.77 (dd, 1H), 2.21 (s, 3H), 1.57 (s, 9H)

### Step 2: Preparation of 4-pyridazin-4-ylthiadiazole

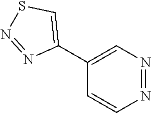 [SMILES: c1cc(-c2csnn2)cnn1]

A solution of tert-butyl N-[(E)-1-pyridazin-4-ylethylideneamino]carbamate (0.25 g) in dichloromethane (4 mL), under nitrogen atmosphere, was cooled to −78° C. and thionyl chloride (0.386 mL) was added dropwise. The reaction was allowed to slowly warm to room temperature. The reaction was diluted with water and extracted with dichloromethane (2×). The combined organic layers were concentrated and purified by silica gel chromatography eluting with 0 to 100% ethyl acetate in iso-hexane to afford 4-pyridazin-4-ylthiadiazole.

¹H NMR (400 MHz, CDCl₃) 10.18 (s, 1H), 9.66 (dd, 1H), 9.08 (dd, 1H), 7.57 (s, 1H)

### Step 3: Preparation of 3-[4-(thiadiazol-4-yl)pyridazin-1-ium-1-yl]propanoic acid 2,2,2-trifluoroacetate A139

A mixture of 4-pyridazin-4-ylthiadiazole (0.08 g), water (5 mL) and 3-bromopropanoic acid (0.298 g) was heated at 100° C. for 2.5 hour. The reaction mixture was cooled, concentrated and purified by preparative reverse phase HPLC (trifluoroacetic acid was present in the eluent) to afford 3-[4-(thiadiazol-4-yl)pyridazin-1-ium-1-yl]propanoic acid 2,2,2-trifluoroacetate.

¹H NMR (400 MHz, CD₃OD) 10.26 (d, 1H), 10.23 (s, 1H), 9.97 (d, 1H), 9.24 (dd, 1H), 5.13 (t, 2H), 3.27 (t, 2H) (CO₂H proton missing)

### Example 25: Preparation of 5-pyridazin-4-ylisoxazole

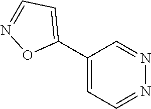 [SMILES: c1cc(-c2ccno2)cnn1]

### Step 1: Preparation of (E)-3-(dimethylamino)-1-pyridazin-4-yl-prop-2-en-1-one

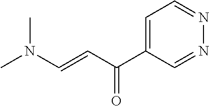

To 1-pyridazin-4-ylethanone (0.230 g) was added 1,1-dimethoxy-N,N-dimethyl-methanamine (0.275 mL) and the mixture was heated at reflux for 1 hour, cooled to room temperature and allowed to stand overnight. The combined organic layers were concentrated and purified by silica gel chromatography eluting with 0 to 50% acetonitrile in dichloromethane to afford (E)-3-(dimethylamino)-1-pyridazin-4-yl-prop-2-en-1-one as an orange solid.

¹H NMR (400 MHz, CDCl₃) 9.57-9.53 (m, 1H), 9.32 (dd, 1H), 7.92 (d, 1H), 7.84 (dd, 1H), 5.66 (d, 1H), 3.24 (s, 3H), 3.00 (s, 3H)

### Step 2: Preparation of 5-pyridazin-4-ylisoxazole

A mixture of (E)-3-(dimethylamino)-1-pyridazin-4-yl-prop-2-en-1-one (0.05 g) and 4M hydrochloric acid in dioxane (1 mL) was heated at reflux for 45 minutes. The mixture was cooled to room temperature and hydroxylamine hydrochloride (0.024 g) was added and then heated at reflux for 12 hours. The reaction mixture was concentrated, dissolved in water (10 mL) and extracted with ethyl acetate (3×30 mL). The combined organic layers were dried over sodium sulfate, concentrated and purified by silica gel chromatography eluting with 0 to 50% methanol in dichloromethane to afford 5-pyridazin-4-ylisoxazole.

¹H NMR (400 MHz, CD₃OD) 9.64-9.70 (m, 1H), 9.35 (d, 1H), 8.61 (d, 1H), 8.15 (d, 1H), 7.29 (d, 1H)

### Example 26: Preparation of 2-methyl-5-pyridazin-4-y-1,3,4-thiadiazole

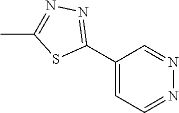

### Step 1: Preparation of pyridazine-4-carbohydrazide

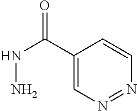

To a solution of methyl pyridazine-4-carboxylate (0.4 g) in methanol (4.92 mL) was added hydrazine hydrate (1.12 mL) and the mixture was heated at reflux for 26 hours. The reaction mixture was cooled to room temperature and concentrated to give pyridazine-4-carbohydrazide as a brown solid.

¹H NMR (400 MHz, CD₃OD) 9.52-9.48 (m, 1H), 9.36 (dd, 1H), 8.00 (dd, 1H) (NH protons missing)

### Step 2: Preparation of N′-acetylpyridazine-4-carbohydrazide

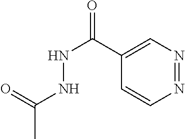

A mixture of pyridazine-4-carbohydrazide (2.3 g), acetic acid (23 mL) and acetic anhydride (1.9 mL) was heated at 100° C. for 16 hours. The reaction mass was concentrated and the resulting solid was washed with tert-butyl methyl ether (2×20 mL) and ethyl acetate (2×20 mL) and dried under vacuum to give N′-acetylpyridazine-4-carbohydrazide.

¹H NMR (400 MHz, DMSO-d₆) 10.87 (s, 1H), 10.12 (s, 1H), 9.54 (dd, 1H), 9.47 (dd, 1H), 8.02 (dd, 1H), 1.94 (s, 3H)

### Step 3: Preparation of 2-methyl-5-pyridazin-4-yl-1,3,4-thiadiazole

A microwave vial was charged with N′-acetylpyridazine-4-carbohydrazide (0.1 g), 1,4-Dioxane (1 mL), phosphorus pentasulfide (0.123 g) and aluminium oxide (0.084 g) and heated at 140° C. for 1 hour. The reaction mass was quenched in ice cold water and extracted with ethyl acetate. The organic layer was dried over sodium sulfate, concentrated and purified by silica gel chromatography eluting with 0 to 15% methanol in dichloromethane to afford 2-methyl-5-pyridazin-4-yl-1,3,4-thiadiazole.

¹H NMR (400 MHz, DMSO-d₆) 9.76 (dd, 1H), 9.43 (dd, 1H), 8.18 (dd, 1H), 2.85 (s, 3H)

### Example 27: Preparation of 3-[4-(1,2,4-thiadiazol-3-yl)pyridazin-1-ium-1-yl]propanoic acid 2,2,2-trifluoroacetate A142

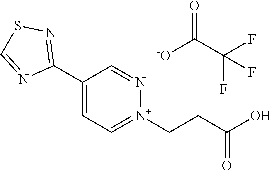

### Step 1: Preparation of pyridazine-4-carboxamidine hydrochloride

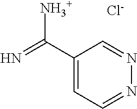

To a mixture of pyridazine-4-carbonitrile (3.5 g) in methanol (18 mL) was added sodium methoxide (25% in methanol, 0.78 mL) at room temperature and the reaction mixture was stirred for 3 hours. To this mixture was added ammonium chloride (2 g) and stirring was continued at room temperature for a further 18 hours. The reaction mixture was concentrated and the resulting residue was washed with tert-butyl methyl ether to give pyridazine-4-carboxamidine hydrochloride as a brown solid.

¹H NMR (400 MHz, DMSO-d₆) 9.58-9.60 (m, 2H), 8.12-8.14 (m, 1H)

### Step 2: Preparation of 3-pyridazin-4-yl-4H-1,2,4-thiadiazole-5-thione

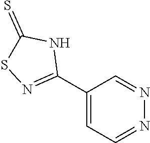 [SMILES: S=c1[nH]c(-c2ccnnc2)ns1]

To a mixture of pyridazine-4-carboxamidine hydrochloride (50 mg), carbon disulfide (0.5M in THF, 2 mL), sulfur (0.013 g) and methanol (0.5 mL) was added sodium methoxide (25% in methanol, 0.144 mL) and the reaction was heated at 60° C. for 5 hours. The reaction mixture was concentrated and purified by silica gel chromatography eluting with ethyl acetate in methanol to give 3-pyridazin-4-yl-4H-1,2,4-thiadiazole-5-thione as dark brown solid.

¹H NMR (400 MHz, DMSO-d₆) 9.73 (s, 1H) 9.33 (dd, 1H) 8.12 (dd, 1H)

### Step 3: Preparation of 3-pyridazin-4-yl-1,2,4-thiadiazole

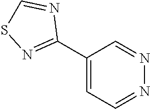 [SMILES: c1cc(-c2ncsn2)cnn1]

A mixture of 3-pyridazin-4-yl-4H-1,2,4-thiadiazole-5-thione (0.5 g) and acetic acid (12.74 mL) was cooled to 15° C. and hydrogen peroxide (1.56 mL) was added drop wise. The mixture was stirred at room temperature for 3 hours, when further hydrogen peroxide (1.56 mL) was added. After a further 2 hours stirring the reaction mixture was quenched with sodium metabisulfite solution, neutralised and extracted with ethyl acetate (3×50 mL). The combined organic phases were dried over anhydrous sodium sulfate and purified by silica gel chromatography eluting with ethyl acetate to give 3-pyridazin-4-yl-1,2,4-thiadiazole.

¹H NMR (400 MHz, CDCl₃) 10.09 (dd, 1H), 10.01 (s, 1H), 9.41 (dd, 1H), 8.33 (dd, 1H)

### Step 4: Preparation of 3-[4-(1,2,4-thiadiazol-3-yl)pyridazin-1-ium-1-yl]propanoic acid 2,2,2-trifluoroacetate A142

To a solution of 3-pyridazin-4-yl-1,2,4-thiadiazole (0.2 g) in acetonitrile (8 mL) was added 3-bromopropanoic acid (0.224 g) and the mixture heated at reflux for 30 hours. The reaction mixture was cooled, concentrated and purified by preparative reverse phase HPLC (trifluoroacetic acid was present in the eluent) to afford 3-[4-(1,2,4-thiadiazol-3-yl)pyridazin-1-ium-1-yl]propanoic acid 2,2,2-trifluoroacetate.

¹H NMR (400 MHz, D₂O) 10.35 (s, 1H), 10.19 (d, 1H), 9.90 (d, 1H), 9.18 (dd, 1H), 5.14 (t, 2H), 3.26 (t, 2H) (CO₂H proton missing)

Additional compounds in Table A (below) were prepared by analogues procedures, from appropriate starting materials. The skilled person would understand that the compounds of formula (I) may exist as an agronomically acceptable salt, a zwitterion or an agronomically acceptable salt of a zwitterion as described hereinbefore. Where mentioned the specific counterion is not considered to be limiting, and the compound of formula (I) may be formed with any suitable counter ion.

NMR spectra contained herein were recorded on a (400 MHz Bruker AVANCE III HD equipped with Bruker SMART probe unless otherwise stated. Chemical shifts are expressed as ppm downfield from TMS, with an internal reference of either TMS or the residual solvent signals. The following multiplicities are used to describe the peaks: s=singlet, d=doublet, t=triplet, dd=double doublet, dt=double triplet, q=quartet, quin=quintet, m=multiplet. Additionally br. is used to describe abroad signal and app. is used to describe an apparent multiplicity.

TABLE A
Physical Data for Compounds of the Invention

| Compound | | |
|---|---|---|
| Number | Structure | ¹H NMR |
| A1 | 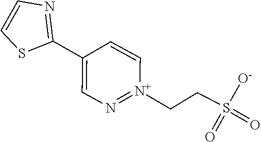 [SMILES: O=S(=O)([O-])CC[n+]1ccc(-c2nccs2)cn1] | (400 MHz, D₂O) 9.84-9.94 (m, 1H) 9.63-9.72 (m, 1H) 8.82 (dd, 1H) 8.14- 8.25 (m, 1H) 8.08 (d, 1H) 5.09-5.19 (m, 2H) 3.54-3.68 (m, 2H) |
| A2 | 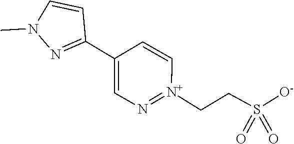 [SMILES: Cn1ccc(-c2cc[n+](CCS(=O)(=O)[O-])nc2)n1] | (400 MHz, D₂O) 9.67 (d, 1H) 9.45 (d, 1H) 8.55 (dd, 1H) 7.71 (d, 1H) 7.09(d, 1H) 5.10-5.01 (m, 2H) 3.94 (s, 3H) 3.64-3.54 (m, 2H) |
| A3 | 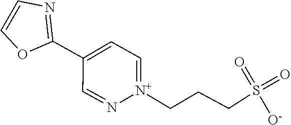 [SMILES: O=S(=O)([O-])CCC[n+]1ccc(-c2ncco2)cn1] | (400 MHz, D₂O) 9.83-9.95 (m, 1H) 9.73 (d, 1H) 8.86 (dd, 1H) 8.08-8.31 (m, 1H) 7.49-7.71 (m, 1H) 4.85-5.08 (m, 2H), 2.85-3.16 (m, 2H) 2.50 (quin, 2H) |
| A4 | 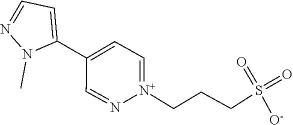 [SMILES: Cn1nccc1-c1cc[n+](CCCS(=O)(=O)[O-])nc1] | (400 MHz, D₂O) 9.52-9.69 (m, 2H) 8.52-8.67 (m, 1H) 7.59-7.70 (m, 1H) 7.03 (d, 1H) 4.92 (t, 2H) 3.93-4.15 (m, 3H) 2.89-3.02 (m, 2H) 2.39-2.62 (m, 2H) |
| A5 | 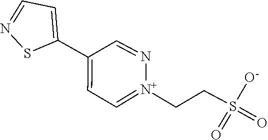 [SMILES: O=S(=O)([O-])CC[n+]1ccc(-c2ccns2)cn1] | (400 MHz, D₂O) 9.73-9.79 (m, 1H) 9.62-9.74 (m, 1H) 8.72-8.81 (m, 1H) 8.58-8.69 (m, 1H) 8.04-8.23 (m, 1H) 5.17 (t, 2H) 3.53-3.77 (m, 2H) |
| A6 | 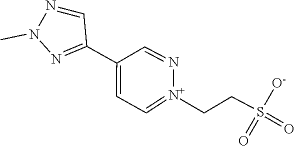 [SMILES: Cn1ncc(-c2cc[n+](CCS(=O)(=O)[O-])nc2)n1] | (400 MHz, D₂O) 9.74-9.92 (m, 1H) 9.54-9.69 (m, 1H) 8.64-8.85 (m, 1H) 8.41-8.60 (m, 1H) 5.09-5.24 (m, 2H) 4.18-4.40 (m, 3H) 3.56-3.76 (m, 2H) |
| A7 | 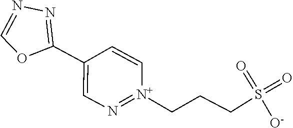 [SMILES: O=S(=O)([O-])CCC[n+]1ccc(-c2nnco2)cn1] | (400 MHz, D₂O) 10.06-10.03 (m, 1H) 9.95-9.92 (m, 1H) 9.24-9.21 (m, 1H) 9.11-9.07 (m, 1H) 5.06-5.01 (m, 2H) 3.03-2.94 (m, 2H) 2.60-2.47 (m, 2H) [isolated as a 1:1 mixture of isomers with 10.50-10.47 (m, 1H) 9.70-9.66 (m, 1H) 9.20-9.17 (m, 1H) 9.03-8.99 (m, 1H) 5.12-5.06 (m, 1H) 3.03-2.94 (m, 2H) 2.60-2.47 (m, 2H)] |
| A8 | 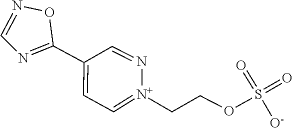 [SMILES: O=S(=O)([O-])OCC[n+]1ccc(-c2ncno2)cn1] | (400 MHz, DMSO-d₆) 10.32-10.28 (m, 1H) 10.18-10.12 (m, 1H) 9.55-9.54 (m, 1H) 9.43-9.38 (m, 1H) 5.26-5.14 (m, 2H) 4.43-4.33 (m, 2H) [isolated as a 1:1 mixture of isomers with 10.72 (s, 1H) 9.95-9.90 (m, 1H) 9.53-9.52 (m, 1H) 9.27-9.22 (m, 1H) 5.26-5.14 (m, 2H) 4.43-4.33 (m, 2H)] |
| A9 | 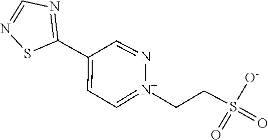 [SMILES: O=S(=O)([O-])CC[n+]1ccc(-c2ncns2)cn1] | (400 MHz, D₂O) 10.00-10.06 (m, 1H) 9.91 (d, 1H) 9.07 (dd, 1H) 8.94-9.13 (m, 1H) 5.17-5.28 (m, 2H) 3.62-3.71 (m, 2H) |
| A10 | 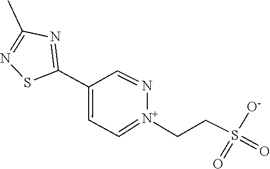 [SMILES: Cc1nsc(-c2cc[n+](CCS(=O)(=O)[O-])nc2)n1] | (400 MHz, D₂O) 9.92-9.97 (m, 1H) 9.83-9.90 (m, 1H) 8.96-9.01 (m, 1H) 5.15-5.27 (m, 2H) 3.56-3.70 (m, 2H) 2.71 (s, 3H) |
| A11 | 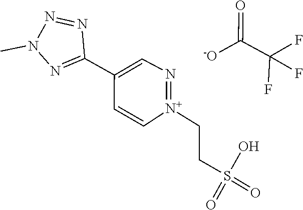 | (400 MHz, D₂O) 10.09 (d, 1H) 9.89 (d, 1H) 8.98-9.15 (m, 1H) 5.10-5.39 (m, 2H) 4.51 (s, 3H) 3.49-3.75 (m, 2H) (one SO₃H proton missing) |
| A12 | 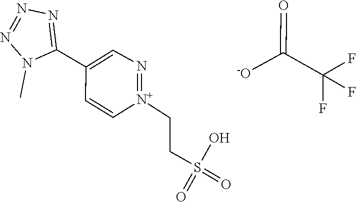 | (400 MHz, D₂O) 10.01-10.08 (m, 2H) 9.10 (d, 1H) 5.30-5.38 (m, 2H) 4.40 (s, 3H) 3.69-3.77 (m, 2H) (one SO₃H proton missing) |
| A13 | 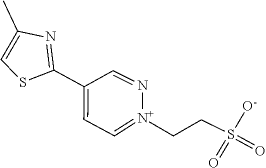 [SMILES: Cc1csc(-c2cc[n+](CCS(=O)(=O)[O-])nc2)n1] | (400 MHz, D₂O) 9.86 (d, 1H) 9.66 (d, 1H) 8.78 (dd, 1H) 7.72 (s, 1H) 5.16 (t, 2H) 3.66 (t, 2H) 2.52 (s, 3H) |
| A14 | 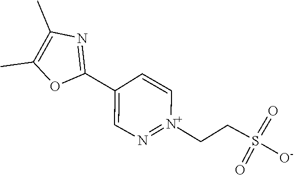 [SMILES: Cc1nc(-c2cc[n+](CCS(=O)(=O)[O-])nc2)oc1C] | (400 MHz, D₂O) 9.74-9.78 (m, 1H) 9.66 (d, 1H) 8.68 (dd, 1H) 5.11-5.18 (m, 2H) 3.61-3.68 (m, 2H) 2.40 (s, 3H) 2.18 (s, 3H) |
| A15 | 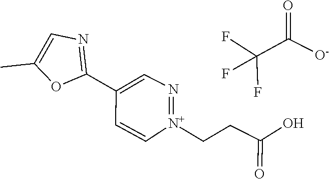 [SMILES: Cc1cnc(-c2cc[n+](CCC(=O)O)nc2)o1 ; O=C([O-])C(F)(F)F] | (400 MHz, D₂O) 9.82 (d, 1H) 9.74 (d, 1H) 8.77 (dd, 1H) 7.29 (d, 1H) 5.06 (t, 2H) 3.23 (t, 2H) 2.47 (d, 3H) (one CO₂H proton missing) |
| A16 | 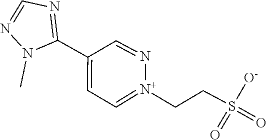 [SMILES: Cn1ncnc1-c1cc[n+](CCS(=O)(=O)[O-])nc1] | (400 MHz, D₂O) 9.88-9.92 (m, 2H) 8.92 (dd, 1H) 8.21 (s, 1H) 5.20-5.23 (m, 2H) 4.17 (s, 3H) 3.63-3.75 (m, 2H) |
| A17 | 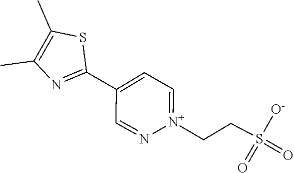 [SMILES: Cc1nc(-c2cc[n+](CCS(=O)(=O)[O-])nc2)sc1C] | (400 MHz, D₂O) 9.76 (s, 1H) 9.57 (d, 1H) 8.65 (d, 1H) 5.13 (t, 2H) 3.66 (t, 2H) 2.51 (s, 3H) 2.42 ppm (s, 3H) |
| A18 | 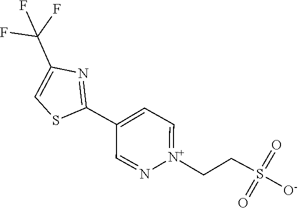 [SMILES: O=S(=O)([O-])CC[n+]1ccc(-c2nc(C(F)(F)F)cs2)cn1] | (400 MHz, D₂O) 9.96 (m, 1H) 9.77 (d, 1H) 8.93 (dd, 1H) 8.60 (s, 1H) 5.16 (t, 2H) 3.64 (t, 2H) |
| A19 | 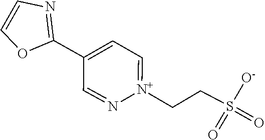 [SMILES: O=S(=O)([O-])CC[n+]1ccc(-c2ncco2)cn1] | 400 MHz, D₂O) 10.01-9.93 (m, 1H) 9.88-9.78 (m, 1H) 8.97-8.90 (m, 1H) 8.33-8.26 (m, 1H) 7.69-7.62 (m, 1H) 5.29-5.19 (m, 2H) 3.75-3.65 (m, 2H) |
| A20 | 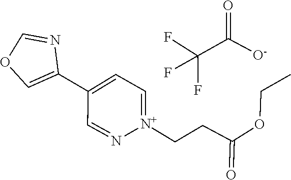 [SMILES: CCOC(=O)CC[n+]1ccc(-c2cocn2)cn1 ; O=C([O-])C(F)(F)F] | (400 MHz, CD₃OD) 9.89 (s, 1H) 9.78 (d, 1H) 9.16 (s, 1H) 8.83 (dd, 1H) 8.52 (s, 1H) 5.07 (t, 2H) 4.14 (q, 2H) 3.24 (t, 2H) 1.23 (t, 3H) |
| A21 | 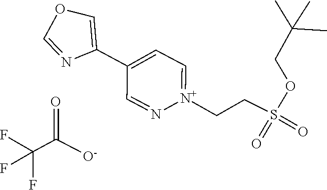 [SMILES: O=C([O-])C(F)(F)F ; CC(C)(C)COS(=O)(=O)CC[n+]1ccc(-c2cocn2)cn1] | (400 MHz, DMSO-d₆) 10.04 (d, 1H) 9.97 (d, 1H) 9.45 (s, 1H) 8.99-8.94 (m, 1H) 8.89 (d, 1H) 5.24 (s, 2H) 4.26 (t, 2H) 3.95 (s, 2H) 0.91 (s, 9H) |
| A22 | 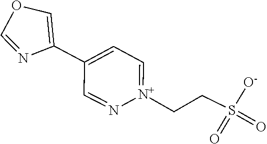 [SMILES: O=S(=O)([O-])CC[n+]1ccc(-c2cocn2)cn1] | (400 MHz, D₂O) 9.70-9.77 (m, 1H) 9.58 (d, 1H) 8.88-8.97 (m, 1H) 8.63- 8.70 (m, 1H) 8.35-8.42 (m, 1H) 5.04- 5.18 (m, 2H) 3.53-3.71 (m, 2H) |
| A23 | 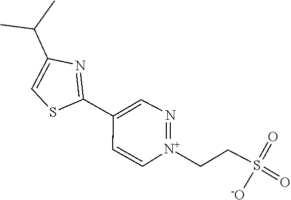 | (400 MHz, D₂O) 9.84 (m, 1H) 9.60 (m, 1H) 8.75 (br d, 1H) 7.71 (br s, 1H) 5.11 (br s, 2H) 3.61 (br s, 2H) 3.16 (br s, 1H) 1.25 (d, 6H) |
| A24 | 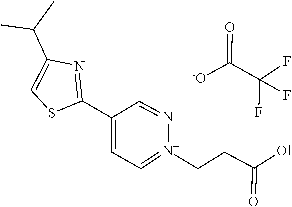 | (400 MHz, CDCl₃) 9 86 (s, 1H) 9.67 (m, 1H) 8.79 (m, 1H) 7.75 (m, 1H) 5.01 (m, 2H) 3.17 (br t, 3H) 1.31 (d, 6H) (one CO₂H proton missing) |
| A25 | 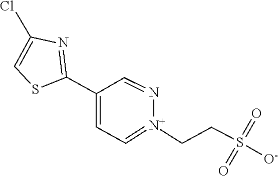 [SMILES: O=S(=O)([O-])CC[n+]1ccc(-c2nc(Cl)cs2)cn1] | (400 MHz, D₂O) 9.88 (d, 1H) 9.73 (d, 1H) 8.83 (dd, 1H) 7.96 (s, 1H) 5.18 (t, 2H) 3.66 (t, 2H) |
| A26 | 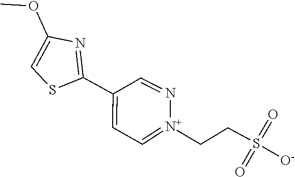 [SMILES: COc1csc(-c2cc[n+](CCS(=O)(=O)[O-])nc2)n1] | (400 MHz, D₂O) 9.83 (s, 1H) 9.64 (d, 1H) 8.75 (dd, 1H) 7.11 (s, 1H) 5.12- 5.18 (m, 2H) 3.98 (s, 3H) 3.65-3.69 (m, 2H) |
| A27 | 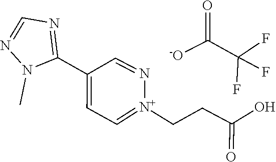 | (400 MHz, D₂O) 9.94 (d, 1H) 9.86-9.90 (m, 1H) 8.93 (dd, 1H) 8.23 (s, 1H) 5.16 (t, 2H) 4.18 (s, 3H) 3.26 (t, 2H) (one CO₂H proton missing) |
| A28 | 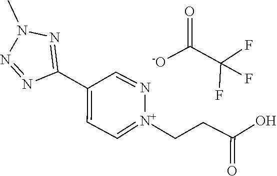 | (400 MHz, D₂O) 10.06 (d, 1H) 9.90 (d, 1H) 9.09 (d, 1H) 5.13 (dd, 2H) 4.51 (br s, 3H) 3.26 (dd, 2H) (one CO₂H proton missing) |
| A29 | 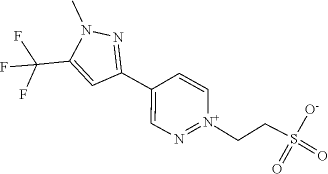 [SMILES: Cn1nc(-c2cc[n+](CCS(=O)(=O)[O-])nc2)cc1C(F)(F)F] | (400 MHz, D₂O) 9.76-9.70 (m, 1H) 9.54 (d, 1H) 8.64 (dd, 1H) 7.57 (s, 1H) 5.09 (t, 2H) 4.05 (s, 3H) 3.59 (t, 2H) |
| A30 | 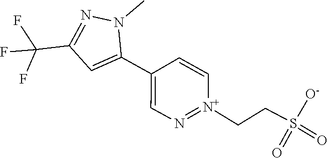 [SMILES: Cn1nc(C(F)(F)F)cc1-c1cc[n+](CCS(=O)(=O)[O-])nc1] | (400 MHz, CD₃OD) 9.91-9.86 (m, 1H) 9.85-9.80 (m, 1H) 8.83 (dd, 1H) 7.54 (s, 1H) 5.27-5.20 (m, 2H) 4.27 (s, 3H) 3.60-3.53 (m, 2H) |
| A31 | 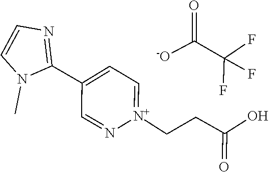 | (400 MHz, D₂O) 9.71-9.90 (m, 2H) 8.67-8.81 (m, 1H) 7.59-7.69 (m, 1H) 7.54 (d, 1H) 5.07 (t, 2H) 3.95-4.07 (m, 3H) 3.13-3.31 (m, 2H) (one CO₂H proton missing) |
| A32 | 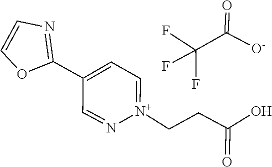 [SMILES: O=C(O)CC[n+]1ccc(-c2ncco2)cn1 ; O=C([O-])C(F)(F)F] | (400 MHz, D₂O) 9.84-9.92 (m, 1H) 9.74-9.82 (m, 1H) 8.77-8.90 (m, 1H) 8.14-8.28 (m, 1H) 7.51-7.62 (m, 1H) 4.94-5.15 (m, 2H) 3.10-3.26 (m, 2H) (one CO₂H proton missing) |
| A33 | 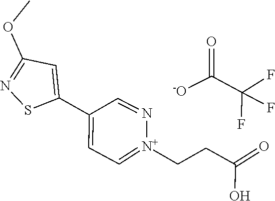 | (400 MHz, D₂O) 9.71 (d, 1H) 9.64 (s, 1H) 8.65 (s, 1H) 7.47 (s, 1H) 5.04 (t, 2H) 3.99 (s, 3H) 3.20 (t, 2H) (one CO₂H proton missing) |
| A34 | 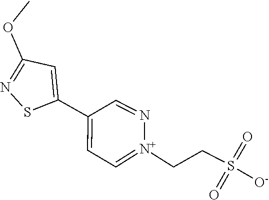 [SMILES: COc1cc(-c2cc[n+](CCS(=O)(=O)[O-])nc2)sn1] | (400 MHz, D₂O) 9.69 (d, 1H) 9.67 (d, 1H) 8.65 (dd, 1H) 7.48 (s, 1H) 5.16 (t, 2H) 3.99 (s, 3H) 3.63 (t, 2H) |
| A35 | 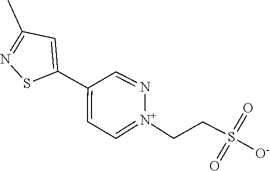 [SMILES: Cc1cc(-c2cc[n+](CCS(=O)(=O)[O-])nc2)sn1] | (400 MHz, D₂O) 9.62 (m, 2H) 9.52 (s, 1H) 8.58 (dd, 1H) 5.14 (t, 2H) 3.63 (t, 2H) 2.61 (s, 3H) |
| A36 | 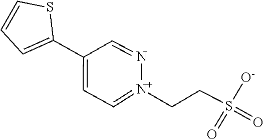 [SMILES: O=S(=O)([O-])CC[n+]1ccc(-c2cccs2)cn1] | (400 MHz, D₂O) 9.63 (d, 1H) 9.38 (d, 1H) 8.47 (dd, 1H) 8.17 (dd, 1H) 8.04 (dd, 1H) 7.36 (dd, 1H) 5.05 (t, 2H) 3.63 ppm (t, 2H) |
| A37 | 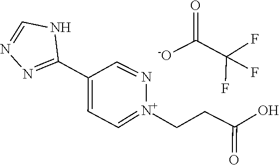 | (400 MHz, D₂O) 9.87 (d, 1H) 9.71 (d, 1H) 8.84 (d, 1H) 8.64 (s, 1H) 5.02 (t, 2H) 3.18 (t, 2H) (one NH proton and one CO₂H proton missing) |
| A38 | 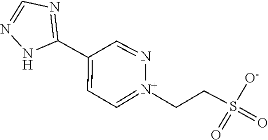 [SMILES: O=S(=O)([O-])CC[n+]1ccc(-c2ncn[nH]2)cn1] | (400 MHz, D₂O) 10.00 (d, 1H) 9.77 (d, 1H) 8.94 (dd, 1H) 8.73 (s, 1H) 5.22 (t, 2H) 3.69 (t, 2H) (one NH proton missing) |
| A39 | 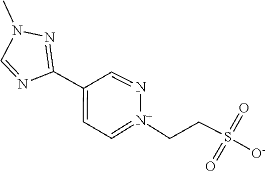 [SMILES: Cn1cnc(-c2cc[n+](CCS(=O)(=O)[O-])nc2)n1] | (400 MHz, D₂O) 9.96 (d, 1H) 9.76 (d, 1H) 8.90 (dd, 1H) 8.62 (s, 1H) 5.22 (t, 2H) 4.06 (s, 3H) 3.69 (t, 2H) |
| A40 | 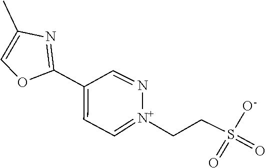 [SMILES: Cc1coc(-c2cc[n+](CCS(=O)(=O)[O-])nc2)n1] | (400 MHz, D₂O) 9.86 (d, 1H) 9.77 (d, 1H) 8.82 (dd, 1H) 8.00 (s, 1H) 5.20 (t, 2H) 3.67 (t, 2H) 2.27 (s, 3H) |
| A41 | 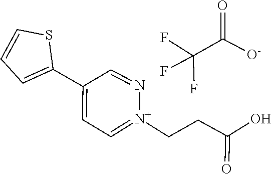 [SMILES: O=C(O)CC[n+]1ccc(-c2cccs2)cn1 ; O=C([O-])C(F)(F)F] | (400 MHz, D₂O) 9.61 (d, 1H) 9.41 (m, 1H) 8.47 (m, 1H) 8.16 (m, 1H) 8.05 (d, 1H) 7.37 (m, 1H) 5.01-4.89 (m, 2H) 3.23-3.12 (m, 2H) (one CO₂H proton missing) |
| A42 | 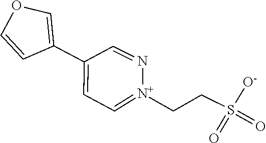 [SMILES: O=S(=O)([O-])CC[n+]1ccc(-c2ccoc2)cn1] | (400 MHz, D₂O) 9.55 (d, 1H) 9.40 (d, 1H) 8.53 (d, 1H) 8.42 (dd, 1H) 7.70 (s, 1H) 7.05 (dd, 1H) 5.04 (m, 2H) 3.60 (m, 2H) |
| A43 | 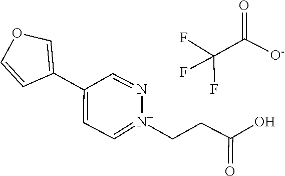 [SMILES: O=C(O)CC[n+]1ccc(-c2ccoc2)cn1 ; O=C([O-])C(F)(F)F] | (400 MHz, D₂O) 9.57 (d, 1H) 9.46 (d, 1H) 8.56 (s, 1H) 8.47 (dd, 1H) 7.74 (m, 1H) 7.09 (dd, 1H) 4.97 (t, 2H) 3.22 (t, 2H) (one CO₂H proton missing) |
| A44 | 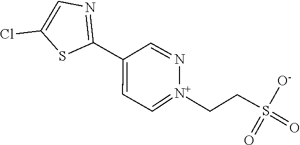 [SMILES: O=S(=O)([O-])CC[n+]1ccc(-c2ncc(Cl)s2)cn1] | (400 MHz, D₂O) 9.90 (d, 1H) 9.73 (d, 1H) 8.81 (dd, 1H) 8.13 (s, 1H) 5.17- 5.22 (m, 2H) 3.66-3.71 (m, 2H) |
| A45 | 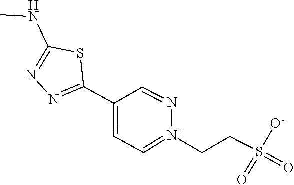 [SMILES: CNc1nnc(-c2cc[n+](CCS(=O)(=O)[O-])nc2)s1] | (400 MHz, D₂O) 9.82 (d, 1H) 9.63 (d, 1H) 8.58 (dd, 1H) 5.10-5.19 (m, 2H) 3.62-3.69 (m, 2H) 3.08 (s, 3H) (one NH proton missing) |
| A46 | 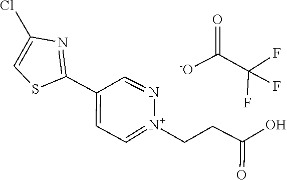 | (400 MHz, D₂O) 9.89 (s, 1H) 9.77 (d, 1H) 8.86 (dd, 1H) 7.99 (s, 1H) 5.09 (t, 2H) 3.25 (t, 2H) (one CO₂H proton missing) |
| A47 | 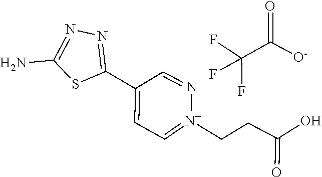 [SMILES: Nc1nnc(-c2cc[n+](CCC(=O)O)nc2)s1 ; O=C([O-])C(F)(F)F] | (400 MHz, D₂O) 9.80 (d, 1H) 9.67 (d, 1H) 8.60 (dd, 1H) 5.04 (t, 2H) 3.24 (t, 2H) (two NH protons and one CO₂H proton missing) |
| A48 | 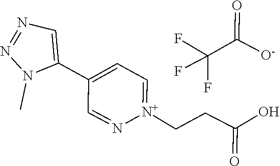 [SMILES: Cn1nncc1-c1cc[n+](CCC(=O)O)nc1 ; O=C([O-])C(F)(F)F] | (400 MHz, D₂O) 9.76-9.82 (m, 1H) 9.65-9.72 (m, 1H) 8.68-8.79 (m, 1H) 8.37-8.43 (m, 1H) 5.02-5.13 (m, 2H) 4.22-4.34 (m, 3H) 3.18-3.25 (m, 2H) (one CO₂H proton missing) |
| A49 | 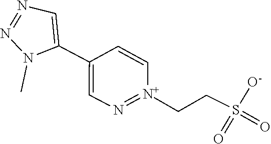 [SMILES: Cn1nncc1-c1cc[n+](CCS(=O)(=O)[O-])nc1] | (400 MHz, D₂O) 9.71-9.96 (m, 2H) 8.76-8.98 (m, 1H) 8.43-8.61 (m, 1H) 5.19-5.42 (m, 2H) 4.29-4.51 (m, 3H) 3.63-3.85 (m, 2H) |
| A50 | 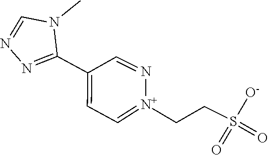 [SMILES: Cn1cnnc1-c1cc[n+](CCS(=O)(=O)[O-])nc1] | (400 MHz, D₂O) 9.95 (d, 1H) 9.90-9.92 (d, 1H) 8.92-8.96 (m, 1H) 8.89 (s, 1H) 5.23-5.35 (m, 2H) 4.04 (s, 3H) 3.67- 3.73 (m, 2H) |
| A51 | 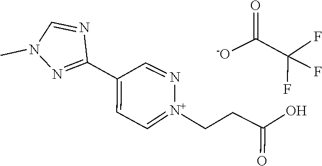 | (400 MHz, D₂O) 9.92 (d, 1H) 9.77 (d, 1H) 8.88 (dd, 1H) 8.61 (s, 1H) 5.10 (t, 2H) 4.05 (s, 3H) 3.26 (t, 2H) (one CO₂H proton missing) |
| A52 | 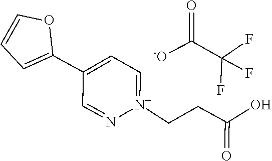 | (400 MHz, D₂O) 9.56 (d, 1H) 9.40 (d, 1H) 8.41 (dd, 1H) 7.98 (d, 1H) 7.73 (d, 1H) 6.82 (dd, 1H) 4.93 (t, 2H) 3.18 (t, 2H) (one CO₂H proton missing) |
| A53 | 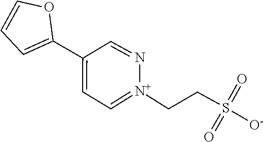 [SMILES: O=S(=O)([O-])CC[n+]1ccc(-c2ccco2)cn1] | (400 MHz, D₂O) 9.58 (d, 1H) 9.38 (d, 1H) 8.41 (dd, 1H) 7.98 (d, 1H) 7.75 (d, 1H) 6.82 (dd, 1H) 5.02-5.07 (m, 2H) 3.60-3.64 (m, 2H) |
| A54 | 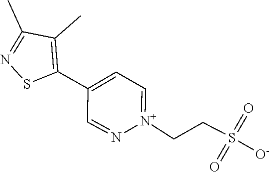 [SMILES: Cc1nsc(-c2cc[n+](CCS(=O)(=O)[O-])nc2)c1C] | (400 MHz, D₂O) 9.73 (dd, 1H) 9.59 (dd, 1H) 8.61 (dd, 1H) 5.20 (t, 2H) 3.67 (t, 2H) 2.44 (s, 3H) 2.35 (s, 3H) |
| A55 | 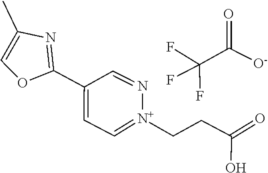 [SMILES: Cc1coc(-c2cc[n+](CCC(=O)O)nc2)n1 ; O=C([O-])C(F)(F)F] | (400 MHz, D₂O) 9.85 (d, 1H) 9.80 (d, 1H) 8.83 (dd, 1H) 8.01 (s, 1H) 5.10 (t, 2H) 3.26 (t, 2H) 2.28 (s, 3H) (one CO₂H proton missing) |
| A56 | 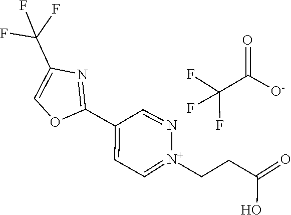 [SMILES: O=C(O)CC[n+]1ccc(-c2nc(C(F)(F)F)co2)cn1 ; O=C([O-])C(F)(F)F] | (400 MHz, D₂O) 9.99 (d, 1H) 9.95 (d, 1H) 9.02 (dd, 1H) 8.80 (s, 1H) 5.15 (t, 2H) 3.26 (t, 2H) (one CO₂H proton missing) |
| A57 | 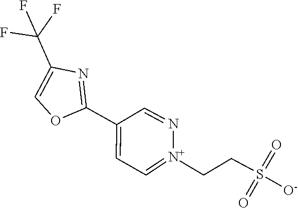 [SMILES: O=S(=O)([O-])CC[n+]1ccc(-c2nc(C(F)(F)F)co2)cn1] | (400 MHz, D₂O) 10.03 (d, 1H) 9.95 (d, 1H) 9.04 (dd, 1H) 8.81 (s, 1H) 5.29 (t, 2H) 3.72 (t, 2H) |
| A58 | 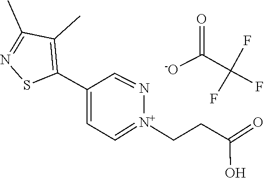 | (400 MHz, D₂O) 9.76 (d, 1H) 9.58 (d, 1H) 8.61 (dd, 1H) 5.09 (t, 2H) 3.24 (t, 2H) 2.45 (s, 3H) 2.35 (s, 3H) (one CO₂H proton missing) |
| A59 | 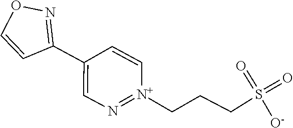 [SMILES: O=S(=O)([O-])CCC[n+]1ccc(-c2ccon2)cn1] | (400 MHz, D₂O) 9.92-9.86 (m, 1H) 9.80-9.71 (m, 1H) 8.89 (dd, 1H) 8.86 (d, 1H) 7.22 (d, 1H) 4.98 (t, 2H) 2.96 (t, 2H) 2.51 (quin, 2H) |
| A60 | 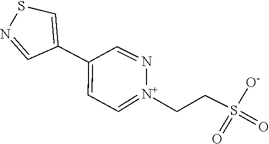 [SMILES: O=S(=O)([O-])CC[n+]1ccc(-c2cnsc2)cn1] | (400 MHz, D₂O) 9.87 (s, 1H) 9.80 (d, 1H) 9.61 (d, 1H) 9.19 (s, 1H) 8.74 (dd, 1H) 5.12-5.17 (m, 2H) 3.63-3.68 (m, 2H) |
| A61 | 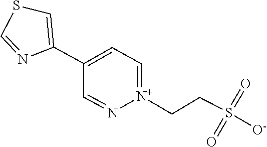 [SMILES: O=S(=O)([O-])CC[n+]1ccc(-c2cscn2)cn1] | (400 MHz, D₂O) 9.91 (d, 1H) 9.62 (d, 1H) 9.21 (d, 1H) 8.79-8.85 (m, 2H) 5.16 (t, 2H) 3.67 (t, 2H) |
| A62 | 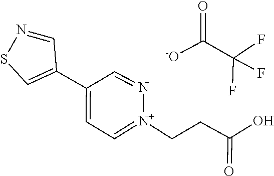 | (400 MHz, D₂O) 9.87 (s, 1H) 9.79 (d, 1H) 9.64 (d, 1H) 9.19 (s, 1H) 8.74 (dd, 1H) 5.04 (t, 2H) 3.24 (t, 2H) (one CO₂H proton missing) |
| A63 | 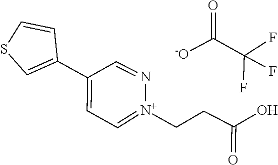 | (400 MHz, D₂O) 9.67 (d, 1H) 9.48 (d, 1H) 8.52-8.58 (m, 2H) 7.75 (d, 1H) 7.68 (dd, 1H) 4.97 (t, 2H) 3.20 (t, 2H) (one CO₂H proton missing) |
| A64 | 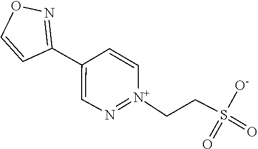 [SMILES: O=S(=O)([O-])CC[n+]1ccc(-c2ccon2)cn1] | (400 MHz, D₂O) 9.91-9.87 (m, 1H) 9.82-9.77 (m, 1H) 8.89 (dd, 1H) 8.85 (d, 1H) 7.22 (d, 1H) 5.19 (t, 2H) 3.65 (t, 2H) |
| A65 | 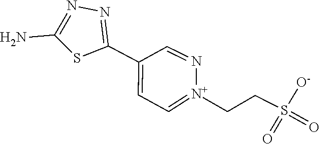 [SMILES: Nc1nnc(-c2cc[n+](CCS(=O)(=O)[O-])nc2)s1] | (400 MHz, D₂O) 9.89 (d, 1H), 9.70 (d, 1H), 8.65-8.68 (m, 1H), 5.22 (t, 2H), 3.67-3.75 ppm (m, 2H) (NH protons missing) |
| A66 | 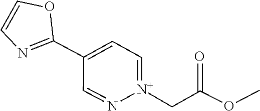 [SMILES: COC(=O)C[n+]1ccc(-c2ncco2)cn1] | (400 MHz, D₂O) 9.92 (s, 1H) 9.76 (d, 1H) 8.94-8.98 (m, 1H) 8.26 (s, 1H) 7.62 (s, 1H) 5.78 (s, 3H) 3.78 (s, 2H) |
| A67 | 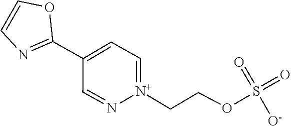 [SMILES: O=S(=O)([O-])OCC[n+]1ccc(-c2ncco2)cn1] | (400 MHz, D₂O) 9.90 (s, 1H) 9.72 (d, 1H) 8.86-8.90 (m, 1H) 8.22 (s, 1H) 7.58 (s, 1H) 5.06-5.12 (m, 2H) 4.52- 4.58 (m, 2H) |
| A68 | 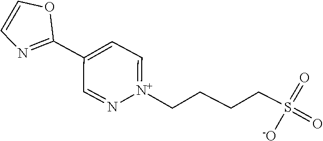 | (400 MHz, D₂O) 9.88 (s, 1H) 9.71 (d, 1H) 8.84-8.88 (m, 1H) 8.20 (s, 1H) 7.56 (s, 1H) 4.80-4.88 (m, 2H) 2.86- 2.92 (m, 2H) 2.14-2.24 (m, 2H) 1.70- 1.78 (m, 2H) |
| A69 | 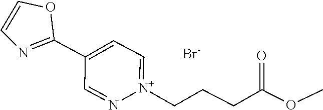 [SMILES: COC(=O)CCC[n+]1ccc(-c2ncco2)cn1 ; [Br-]] | (400 MHz, D₂O) 9.88 (s, 1H) 9.74 (d, 1H) 8.84-8.90 (m, 1H) 8.23 (s, 1H) 7.58 (s, 1H) 4.82-4.88 (m, 2H) 3.59 (s, 3H) 2.46-2.54 (m, 2H) 2.30-2.40 (m, 2H) |
| A70 | 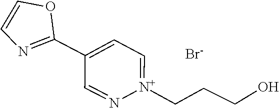 [SMILES: OCCC[n+]1ccc(-c2ncco2)cn1 ; [Br-]] | (400 MHz, D₂O) 9.88 (s, 1H) 9.74 (d, 1H) 8.84-8.88 (m, 1H) 8.21 (s, 1H) 7.58 (s, 1H) 4.88-4.94 (m, 2H) 3.60- 3.68 (m, 2H) 2.22-2.31 (m, 2H) (one OH proton missing) |
| A71 | 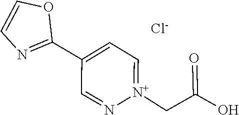 [SMILES: O=C(O)C[n+]1ccc(-c2ncco2)cn1 ; [Cl-]] | (400 MHz, D₂O) 9.86 (d, 1H) 9.66 (d, 1H) 8.90 (dd, 1H) 8.21 (s, 1H) 7.57 (s, 1H) 5.50 (s, 2H) (one CO2H proton missing) |
| A72 | 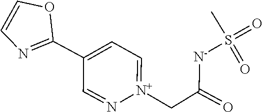 [SMILES: CS(=O)(=O)[N-]C(=O)C[n+]1ccc(-c2ncco2)cn1] | (400 MHz, D₂O) 9.86 (d, 1H) 9.66 (dd, 1H) 8.90 (dd, 1H) 8.22 (s, 1H) 7.63- 7.50 (m, 1H) 5.51-5.44 (m, 2H) 2.98- 2.94 (m, 3H) |
| A73 | 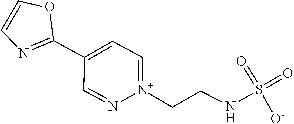 [SMILES: O=S(=O)([O-])NCC[n+]1ccc(-c2ncco2)cn1] | (400 MHz, D₂O) 9.94 (d, 1H) 9.82 (d, 1H) 8.93 (dd, 1H) 8.23 (s, 1H) 7.59 (s, 1H) 5.22-5.14 (m, 2H) 3.79-3.73 (m, 2H) (one NH proton missing) |
| A74 | 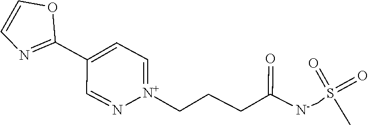 [SMILES: CS(=O)(=O)C[N-]C(=O)CCC[n+]1ccc(-c2ncco2)cn1] | (400 MHz, D₂O) 9.87 (d, 1H) 9.77-9.71 (m, 1H) 8.86 (dd, 1H) 8.21 (s, 1H) 7.57 (s, 1H) 4.90-4.82 (m, 2H) 3.17 (s, 3H) 2.59-2.51 (m, 2H) 2.42-2.32 (m, 2H) |
| A75 | 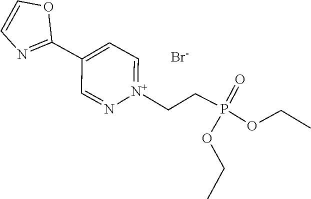 [SMILES: CCOP(=O)(CC[n+]1ccc(-c2ncco2)cn1)OCC ; [Br-]] | (400 MHz, D₂O) 9.97-9.88 (m, 1H) 9.78 (d, 1H) 8.90 (dd, 1H) 8.23 (d, 1H) 7.59 (d, 1H) 5.14-5.00 (m, 2H) 4.05 (qd, 4H) 2.77 (td, 2H) 1.23-1.12 (m, 6H) |
| A76 | 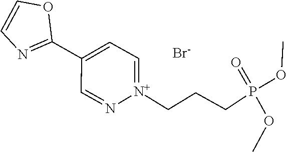 [SMILES: COP(=O)(CCC[n+]1ccc(-c2ncco2)cn1)OC ; [Br-]] | (400 MHz, D₂O) 9.89 (d, 1H) 9.71 (d, 1H) 8.87 (dd, 1H) 8.21 (d, 1H) 7.58 (d, 1H) 4.90-4.83 (m, 2H) 3.69 (s, 3H) 3.66 (s, 3H) 2.40-2.26 (m, 2H) 2.05- 1.93 (m, 2H) |
| A77 | 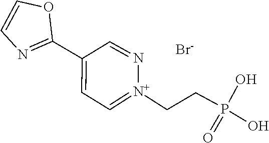 [SMILES: O=P(O)(O)CC[n+]1ccc(-c2ncco2)cn1 ; [Br-]] | (400 MHz, D₂O) 9.87-9.83 (m, 1H) 9.74-9.69 (m, 1H) 8.85-8.80 (m, 1H) 8.20-8.17 (m, 1H) 7.57-7.53 (m, 1H) 5.00-4.90 (m, 2H) 2.42-2.31 (m, 2H) (two OH protons missing) |
| A78 | 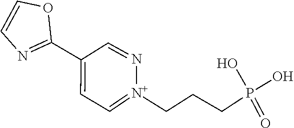 [SMILES: O=P(O)(O)CCC[n+]1ccc(-c2ncco2)cn1] | (400 MHz, D₂O) 9.87 (d, 1H) 9.72 (d, 1H) 8.85 (dd, 1H) 8.20 (s, 1H) 7.56 (s, 1H) 4.86 (br t, 2H) 2.35-2.21 (m, 2H) 1.74-1.59 (m, 2H) (two OH protons missing) |
| A79 | 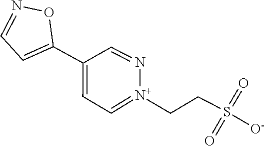 [SMILES: O=S(=O)([O-])CC[n+]1ccc(-c2ccno2)cn1] | (400 MHz, D₂O) 9.92 (d, 1H) 9.85 (d, 1H) 8.91 (dd, 1H) 8.74 (d, 1H) 7.59 (d, 1H) 5.24-5.28 (m, 2H) 3.70-3.74 (m, 2H) |
| A80 | 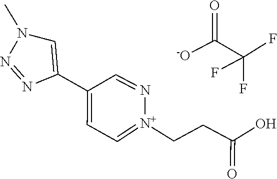 | (400 MHz, D₂O) 9.83 (d, 1H), 9.66 (d, 1H), 8.87 (s, 1H), 8.78 (dd, 1H), 5.06 (t, 2H), 4.22 (s, 3H), 3.25 (t, 2H) (CO₂H proton missing) |
| A81 | 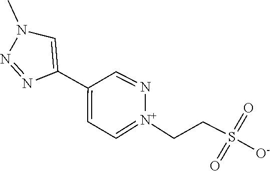 [SMILES: Cn1cc(-c2cc[n+](CCS(=O)(=O)[O-])nc2)nn1] | (400 MHz, D₂O) 9.87 (d, 1H), 9.66 (d, 1H), 8.90 (s, 1H), 8.80 (dd, 1H), 5.19 (m, 2H), 4.24 (s, 3H), 3.70 (m, 2H) |
| A82 | 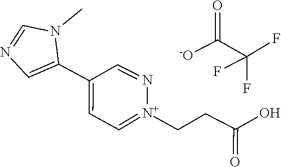 | (400 MHz, D₂O) 9.87 (d, 1H), 9.74 (d, 1H), 9.09 (s, 1H), 8.81 (dd, 1H), 8.36 (s, 1H), 5.14 (t, 2H), 4.13 (s, 3H), 3.29 (t, 2H) (CO₂H proton missing) |
| A83 | 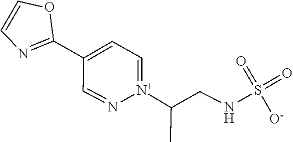 [SMILES: CC(CNS(=O)(=O)[O-])[n+]1ccc(-c2ncco2)cn1] | (400 MHz, D₂O) 9.89 (d, 1H) 9.63 (d, 1H) 8.84 (dd, 1H) 8.23-8.18 (m, 1H) 7.57 (s, 1H) 5.18-5.08 (m, 1H) 3.53- 3.33 (m, 2H) 1.64 (d, 3H) (one NH proton missing) |
| A84 | 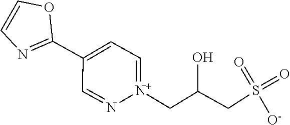 [SMILES: O=S(=O)([O-])CC(O)C[n+]1ccc(-c2ncco2)cn1] | (400 MHz, D₂O) 9.93-9.88 (m, 1H) 9.70-9.66 (m, 1H) 8.92-8.86 (m, 1H) 8.24- 8.21 (m, 1H) 7.60-7.57 (m, 1H) 5.16-5.07 (m, 1H) 4.88-4.78 (m, 2H) 3.25-3.15 (m, 2H) (one OH proton missing) |
| A85 | 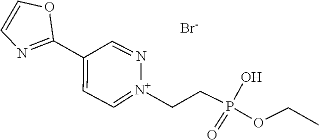 [SMILES: CCOP(=O)(O)CC[n+]1ccc(-c2ncco2)cn1 ; [Br-]] | (400 MHz, D₂O) 9.92-9.87 (m, 1H) 9.78-9.71 (m, 1H) 8.91-8.84 (m, 1H) 8.22 (s, 1H) 7.58 (d, 1H) 5.00-4.90 (m, 2H) 3.86-3.73 (m, 2H) 2.45-2.33 (m, 2H) 1.13-1.07 (m, 3H) (one OH proton missing) |
| A86 | 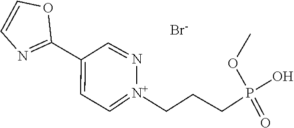 [SMILES: COP(=O)(O)CCC[n+]1ccc(-c2ncco2)cn1 ; [Br-]] | (400 MHz, D₂O) 9.88-9.83 (m, 1H), 9.73-9.67 (m, 1H), 8.88-8.80 (m, 1H), 8.19 (s, 1H), 7.55 (s, 1H), 4.88- 4.78 (m, 2H), 3.46 (d, J = 10.5 Hz, 3H), 2.35-2.17 (m, 2H), 1.74-1.57 (m, 2H) (one OH proton missing) |
| A87 | 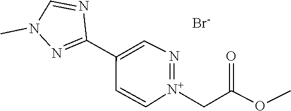 | (400 MHz, D₂O) 9.90 (d, 1H) 9.68 (dd, 1H) 8.92 (dd, 1H) 8.56 (s, 1H) 5.75 (s, 2H) 4.00 (s, 3H) 3.76 (s, 3H) |
| A88 | 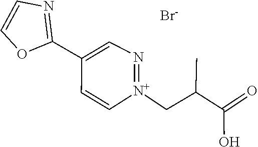 [SMILES: CC(C[n+]1ccc(-c2ncco2)cn1)C(=O)O ; [Br-]] | (400 MHz, D₂O) 9.84 (d, 1H) 9.66 (d, 1H) 8.86-8.80 (m, 1H) 8.19 (s, 1H) 7.55 (s, 1H) 4.95-4.76 (m, 2H) 3.18- 3.06 (m, 1H) 1.18 (d, 3H) (one CO₂H proton missing) |
| A89 | 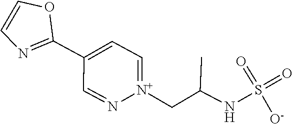 [SMILES: CC(C[n+]1ccc(-c2ncco2)cn1)NS(=O)(=O)[O-]] | (400 MHz, D₂O) 9.87-9.82 (m, 1H) 9.65-9.58 (m, 1H) 8.88-8.81 (m, 1H) 8.20 (s, 1H) 7.57 (s, 1H) 4.96-4.88 (m, 1H) 4.58-4.49 (m, 1H) 3.87-3.74 (m, 1H) 1.31 (d, 3H) (one NH proton missing) |
| A90 | 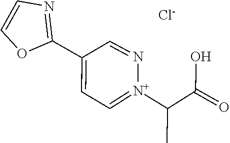 [SMILES: CC(C(=O)O)[n+]1ccc(-c2ncco2)cn1 ; [Cl-]] | (400 MHz, D₂O) 9.92-9.89 (m, 1H) 9.78-9.73 (m, 1H) 8.94-8.89 (m, 1H) 8.26-8.24 (m, 1H) 7.62-7.60 (m, 1H) 5.77 (q, 1H) 1.91 (d, 3H) (one CO₂H proton missing) |
| A91 | 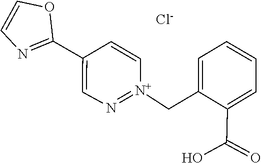 [SMILES: O=C(O)c1ccccc1C[n+]1ccc(-c2ncco2)cn1 ; [Cl-]] | (400 MHz, D₂O) 9.74 (d, 1H) 9.63 (d, 1H) 8.79 (dd, 1H) 8.18 (s, 1H) 8.03 (dd, 1H) 7.68-7.53 (m, 4H) 6.22 (s, 2H) (one CO₂H proton missing) |
| A92 | 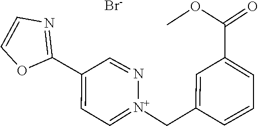 [SMILES: [Br-] ; COC(=O)c1cccc(C[n+]2ccc(-c3ncco3)cn2)c1] | (400 MHz, DMSO-d₆) 10.21 (d, 1H) 10.06 (d, 1H) 9.11 (dd, 1H) 8.72 (s, 1H) 8.24 (s, 1H) 8.02 (d, 1H) 7.88-7.83 (m, 2H) 7.63 (t, 1H) 6.17 (s, 2H) 3.87 (s, 3H) |
| A93 | 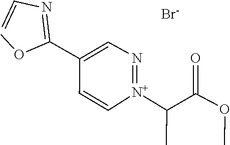 [SMILES: C=C/N=C(\OC)c1cc[n+](C(C)C(=O)OC)nc1 ; [Br-]] | (400 MHz, D₂O) 9.95-9.88 (m, 1H) 9.84-9.75 (m, 1H) 9.00-8.89 (m, 1H) 8.24 (s, 1H) 7.60 (s, 1H) 6.04-5.92 (m, 1H) 3.73 (s, 3H) 1.93 (br d, 3H) |
| A94 | 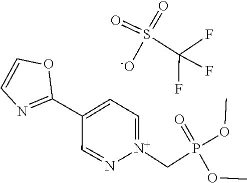 | (400 MHz, D₂O) 9.93 (d, 1H) 9.76 (d, 1H) 8.97-8.91 (m, 1H) 8.25 (s, 1H) 7.61 (s, 1H) 5.62-5.53 (m, 2H) 3.86- 3.80 (m, 6H) |
| A95 | 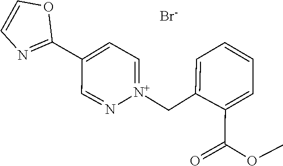 [SMILES: COC(=O)c1ccccc1C[n+]1ccc(-c2ncco2)cn1 ; [Br-]] | (400 MHz, DMSO-d₆) 10.10-10.04 (m, 1H) 10.03-9.99 (m, 1H) 9.09 (dd, 1H) 8.73 (s, 1H) 8.15-7.97 (m, 1H) 7.85 (s, 1H) 7.79-7.70 (m, 1H) 7.70-7.56 (m, 2H) 6.39 (s, 2H) 3.82 (s, 3H) |
| A96 | 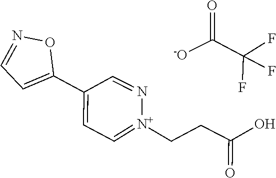 | (400 MHz, D₂O) 9.87 (m, 2H), 8.89 (m, 1H), 8.74 (m, 1H), 7.58 (m, 1H), 5.13 (m, 2H), 3.32 (m,2H) (CO₂H proton missing) |
| A97 | 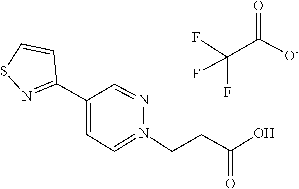 [SMILES: O=C(O)CC[n+]1ccc(-c2ccsn2)cn1 ; O=C([O-])C(F)(F)F] | (400 MHz, D₂O) 10.01 (s, 1H), 9.77 (br d, 1H), 9.16 (d, 1H), 8.93 (dd, 1H), 8.18 (d, 1H), 5.09 (br t, 2H), 3.26 (br t, 2H) (CO₂H proton missing) |
| A98 | 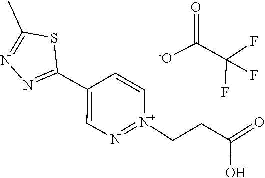 | (400 MHz, D₂O) 10.05 (dd, 1H), 9.95 (d, 1H), 9.02 (dd, 1H), 5.20 (t, 2H), 3.34 (t, 2H), 2.96 (s, 3H) (CO₂H proton missing) |
| A99 | 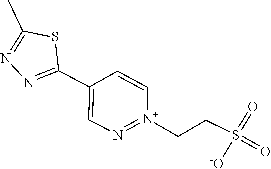 | (400 MHz, D₂O) 9.98 (dd, 1H), 9.83 (d, 1H), 8.93 (dd, 1H), 5.19-5.25 (m, 2H), 3.63-3.70 (m, 2H), 2.87 (s, 3H) |
| A100 | 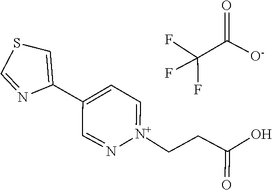 [SMILES: O=C(O)CC[n+]1ccc(-c2cscn2)cn1 ; O=C([O-])C(F)(F)F] | (400 MHz, D₂O) 9.90 (d, 1H), 9.65 (d, 1H), 9.23 (d, 1H), 8.79-8.85 (m, 2H), 5.05 (t, 2H), 3.25 (t, 2H) (CO₂H proton missing) |
| A101 | 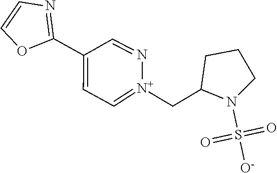 [SMILES: O=S(=O)([O-])N1CCCC1C[n+]1ccc(-c2ncco2)cn1] | (400 MHz, D₂O) 9.83 (d, 1H) 9.59 (d, 1H) 8.84 (dd, 1H) 8.21 (s, 1H) 7.57 (s, 1H) 4.90 (dd, 1H) 4.62-4.52 (m, 1H) 4.13-4.02 (m, 1H) 3.30-3.17 (m, 2H) 2.19-2.04 (m, 1H) 2.01-1.70 (m, 3H) |
| A102 | 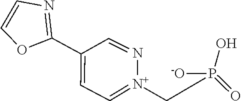 | (400 MHz, D₂O) 9.91-9.86 (m, 1H) 9.69-9.63 (m, 1H) 8.95-8.88 (m, 1H) 8.28-8.22 (m, 1H) 7.64-7.57 (m, 1H) 5.09 (d, 2H) (one POH proton missing) |
| A103 | 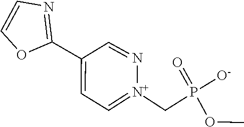 [SMILES: COP(=O)([O-])C[n+]1ccc(-c2ncco2)cn1] | (400 MHz, D₂O) 9.91-9.83 (m, 1H) 9.68-9.58 (m, 1H) 8.97-8.83 (m, 1H) 8.27-8.18 (m, 1H) 7.65-7.54 (m, 1H) 5.18-5.07 (m, 2H) 3.64-3.54 (m, 3H) |
| A104 | 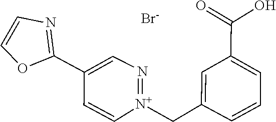 [SMILES: O=C(O)c1cccc(C[n+]2ccc(-c3ncco3)cn2)c1 ; [Br-]] | (400 MHz, D₂O) 9.81 (br d, 2H) 8.86 (dd, 1H) 8.21 (s, 1H) 8.08 (s, 1H) 8.02- 7.95 (m, 1H) 7.78-7.71 (m, 1H) 7.60- 7.56 (m, 1H) 7.52 (s, 1H) 6.04 (s, 2H) (one CO₂H proton missing) |
| A105 | 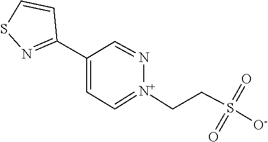 [SMILES: O=S(=O)([O-])CC[n+]1ccc(-c2ccsn2)cn1] | (400 MHz, D₂O) 10.04 (d, 1H), 9.76 (d, 1H), 9.16 (d, 1H), 8.94 (dd, 1H), 8.19 (d, 1H), 5.21 (t, 2H), 3.70 (t, 2H) |
| A106 | 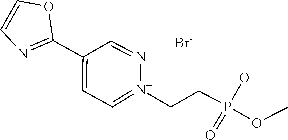 | (400 MHz, D₂O) 9.94-9.91 (m, 1H) 9.79-9.75 (m, 1H) 8.91-8.87 (m, 1H) 8.26-8.23 (m, 1H) 7.62-7.60 (m, 1H) 5.04-4.93 (m, 2H) 3.47 (d, 3H) 2.49- 2.37 (m, 2H) (one POH proton missing) |
| A107 | 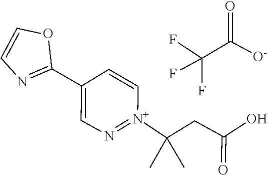 [SMILES: CC(C)(CC(=O)O)[n+]1ccc(-c2ncco2)cn1 ; O=C([O-])C(F)(F)F] | (400 MHz, DMSO-d₆) 10.16-10.10 (m, 2H) 8.98 (dd, 1H) 8.74 (s, 1H) 7.87 (s, 1H) 3.28 (s, 2H) 1.91-1.83 (m, 6H) (one CO₂H proton missing) |
| A108 | 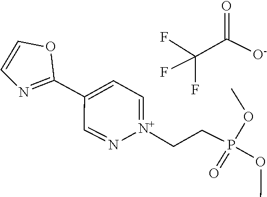 [SMILES: COP(=O)(CC[n+]1ccc(-c2ncco2)cn1)OC ; O=C([O-])C(F)(F)F] | (400 MHz, D₂O) 9.95-9.92 (m, 1H) 9.81-9.77 (m, 1H) 8.94-8.88 (m, 1H) 8.26-8.23 (m, 1H) 7.61 (d, 1H) 5.14- 5.04 (m, 2H) 3.73-3.67 (m, 6H) 2.87- 2.76 (m, 2H) |
| A109 | 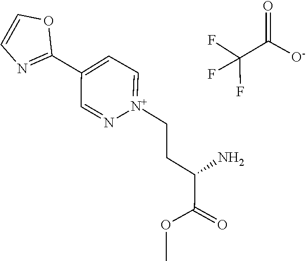 [SMILES: COC(=O)[C@@H](N)CC[n+]1ccc(-c2ncco2)cn1 ; O=C([O-])C(F)(F)F] | (400 MHz, D₂O) 9.95-9.91 (m, 1H) 9.80-9.74 (m, 1H) 8.93-8.88 (m, 1H) 8.24 (s, 1H) 7.62-7.58 (m, 1H) 5.11- 5.04 (m, 2H) 4.32-4.25 (m, 1H) 3.79 (s, 3H) 2.84-2.61 (m, 2H) (two NH protons missing) |
| A110 | 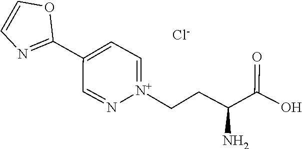 [SMILES: N[C@@H](CC[n+]1ccc(-c2ncco2)cn1)C(=O)O ; [Cl-]] | (400 MHz, D₂O) 9.99-9.86 (m, 1H) 9.78 (d, 1H) 8.90 (dd, 1H) 8.24 (s, 1H) 7.61 (s, 1H) 5.05 (br t, 2H) 4.00-3.92 (m, 1H) 2.74-2.61 (m, 2H) (two NH protons and one CO₂H proton missing) |
| A111 | 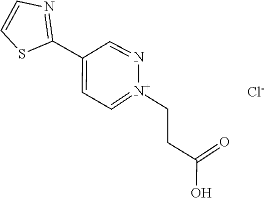 [SMILES: O=C(O)CC[n+]1ccc(-c2nccs2)cn1 ; [Cl-]] | (400 MHz, D₂O) 9.91-9.86 (m, 1H) 9.69 (d, 1H) 8.85-8.80 (m, 1H) 8.21- 8.18 (m, 1H) 8.11-8.07 (m, 1H) 5.03 (t, 2H) 3.21 (t, 2H) (one CO₂H proton missing) |
| A112 | 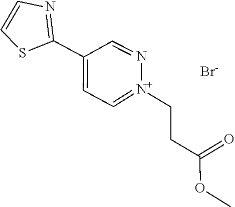 [SMILES: COC(=O)CC[n+]1ccc(-c2nccs2)cn1 ; [Br-]] | (400 MHz, D₂O) 9.89-9.86 (m, 1H) 9.72-9.68 (m, 1H) 8.85-8.80 (m, 1H) 8.20-8.17 (m, 1H) 8.11-8.07 (m, 1H) 5.07-5.01 (m, 2H) 3.60 (s, 3H) 3.26- 3.20 (m, 2H) |
| A113 | 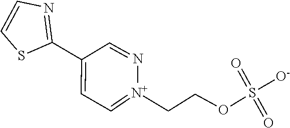 [SMILES: O=S(=O)([O-])OCC[n+]1ccc(-c2nccs2)cn1] | (400 MHz, D₂O) 9.94-9.91 (m, 1H) 9.66-9.63 (m, 1H) 8.89-8.84 (m, 1H) 8.21 (d, 1H) 8.12-8.09 (m, 1H) 5.10- 5.06 (m, 2H) 4.60-4.55 (m, 2H) |
| A114 | 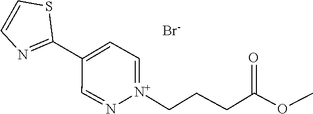 [SMILES: COC(=O)CCC[n+]1ccc(-c2nccs2)cn1 ; [Br-]] | (400 MHz, D₂O) 9.94-9.90 (m, 1H) 9.70-9.65 (m, 1H) 8.90-8.85 (m, 1H) 8.25-8.21 (m, 1H) 8.14-8.11 (m, 1H) 4.89-4.83 (m, 2H) 3.63 (s, 3H) 2.58- 2.51 (m, 2H) 2.43-2.34 (m, 2H) |
| A115 | 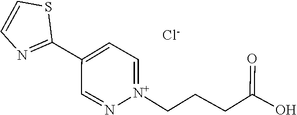 [SMILES: O=C(O)CCC[n+]1ccc(-c2nccs2)cn1 ; [Cl-]] | (400 MHz, D₂O) 9.87 (d, 1H) 9.63 (d, 1H) 8.82 (dd, 1H) 8.19-8.16 (m, 1H) 8.09-8.06 (m, 1H) 4.85-4.79 (m, 2H) 2.56-2.40 (m, 2H) 2.40-2.25 (m, 2H) (one CO₂H proton missing) |
| A116 | 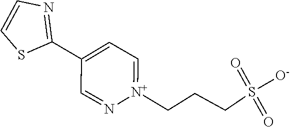 [SMILES: O=S(=O)([O-])CCC[n+]1ccc(-c2nccs2)cn1] | (400 MHz, D₂O) 9.92-9.89 (m, 1H) 9.67-9.63 (m, 1H) 8.87-8.82 (m, 1H) 8.20 (d, 1H) 8.11-8.08 (m, 1H) 4.97- 4.91 (m, 2H) 3.01-2.95 (m, 2H) 2.57- 2.46 (m, 2H) |
| A117 | 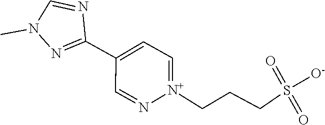 [SMILES: Cn1cnc(-c2cc[n+](CCCS(=O)(=O)[O-])nc2)n1] | (400 MHz, D₂O) 9.90 (d, 1H) 9.68 (d, 1H) 8.85 (dd, 1H) 8.57 (s, 1H) 4.95 (t, 2H) 4.01 (s, 3H) 2.97 (t, 2H) 2.55-2.47 (m, 2H) |
| A118 | 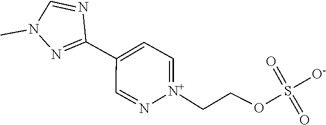 [SMILES: Cn1cnc(-c2cc[n+](CCOS(=O)(=O)[O-])nc2)n1] | (400 MHz, D₂O) 9.95-9.91 (m, 1H) 9.68 (dd, 1H) 8.91-8.87 (m, 1H) 8.58 (s, 1H) 5.13-5.09 (m, 2H) 4.62-4.57 (m, 2H) 4.03 (s, 3H) |
| A119 | 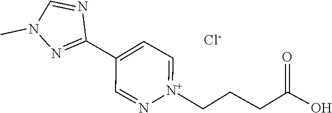 [SMILES: Cn1cnc(-c2cc[n+](CCCC(=O)O)nc2)n1 ; [Cl-]] | (400 MHz, D₂O) 9.92-9.89 (m, 1H) 9.72-9.68 (m, 1H) 8.89-8.85 (m, 1H) 8.59 (s, 1H) 4.91-4.85 (m, 2H) 4.03 (s, 3H) 2.55-2.50 (m, 2H) 2.41-2.32 (m, 2H) (one CO₂H proton missing) |
| A120 | 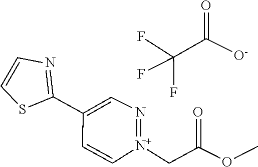 [SMILES: COC(=O)C[n+]1ccc(-c2nccs2)cn1 ; O=C([O-])C(F)(F)F] | (400 MHz, D₂O) 9.92 (d, 1H) 9.66 (dd, 1H) 8.92 (dd, 1H) 8.22 (d, 1H) 8.13 (d, 1H) 5.75 (s, 2H) 3.79 (s, 3H) |
| A121 | 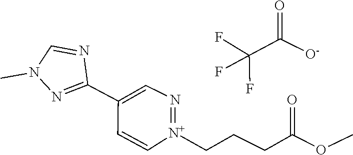 | (400 MHz, D₂O) 9.91 (d, 1H) 9.71 (d, 1H) 8.89 (dd, 1H) 8.61 (s, 1H) 4.88 (t, 2H) 4.05 (s, 3H) 3.63 (s, 3H) 2.55 (t, 2H) 2.44-2.34 (m, 2H) |
| A122 | 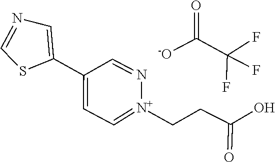 | (400 MHz, D₂O) 9.74 (d, 1H), 9.61 (d, 1H), 9.39 (s, 1H) 8.84 (s, 1H), 8.65 (dd, 1H), 5.04 (t, 2H), 3.25 (t, 2H) (CO₂H proton missing) |
| A123 | 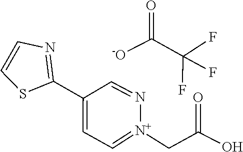 | (400 MHz, CD₃OD) 10.11 (d, 1H), 9.80 (dd, 1H), 9.09 (dd, 1H), 8.34 (d, 1H), 8.28 (d, 1H), 5.71 (s, 2H) (CO₂H proton missing) |
| A124 | 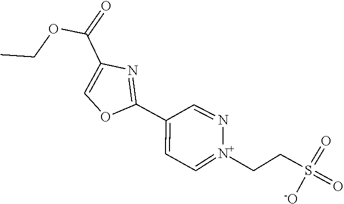 | (400 MHz, D₂O) 10.01-9.98 (m, 1H), 9.91-9.87 (m, 1H), 9.03-8.98 (m, 1H), 8.85 (s, 1H), 5.28-5.22 (m, 2H), 4.40 (q, 2H), 3.72-3.66 (m, 2H), 1.34 (t, 3H) |
| A125 | 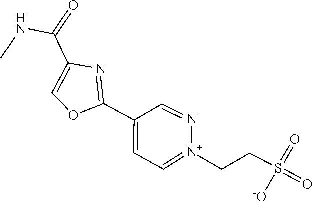 | (400 MHz, D₂O) 9.99 (s, 1H), 9.86 (d, 1H), 9.01-8.96 (m, 1H), 8.68 (s, 1H), 5.27-5.21 (m, 2H), 4.76 (s, 3H), 3.72- 3.66 (m, 2H) (NH proton missing) |
| A126 | 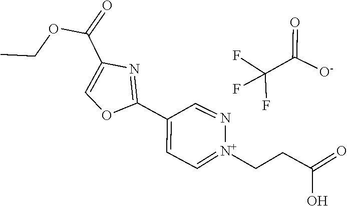 [SMILES: CCOC(=O)c1coc(-c2cc[n+](CCC(=O)O)nc2)n1 ; O=C([O-])C(F)(F)F] | (400 MHz, D₂O) 9.94 (s, 1H), 9.89- 9.85 (m, 1H), 8.98-8.93 (m, 1H), 8.81 (s, 1H), 5.13-5.07 (m, 2H), 4.37 (q, 2H), 3.27-3.21 (m, 2H), 1.34-1.27 (m, 3H) (CO₂H proton missing) |
| A127 | 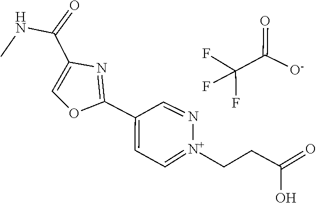 [SMILES: CNC(=O)c1coc(-c2cc[n+](CCC(=O)O)nc2)n1 ; O=C([O-])C(F)(F)F] | (400 MHz, D₂O) 9.94-9.91 (m, 1H), 9.86-9.82 (m, 1H), 8.95-8.91 (m, 1H), 8.64 (s, 1H), 5.10-5.05 (m, 2H), 3.23- 3.18 (m, 2H), 2.87 (s, 3H) (NH and CO₂H protons missing) |
| A128 | 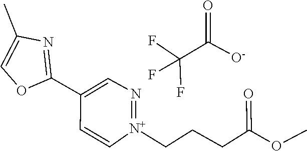 [SMILES: COC(=O)CCC[n+]1ccc(-c2nc(C)co2)cn1 ; O=C([O-])C(F)(F)F] | (400 MHz, D₂O) 9.84 (s, 1H), 9.74- 9.69 (m, 1H), 8.85-8.79 (m, 1H), 7.99 (s, 1H), 4.90-4.84 (m, 2H), 3.62 (s, 3H), 2.57-2.51 (m, 2H), 2.43-2.34 (m, 2H), 2.26 (s, 3H) |
| A129 | 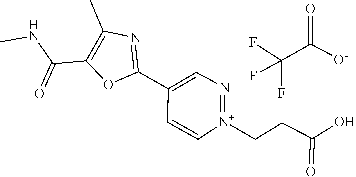 [SMILES: CNC(=O)c1oc(-c2cc[n+](CCC(=O)O)nc2)nc1C ; O=C([O-])C(F)(F)F] | (400 MHz, D₂O) 9.87-9.85 (m, 1H), 9.85-9.82 (m, 1H), 8.89-8.85 (m, 1H), 5.11-5.06 (m, 2H), 3.25-3.20 (m, 2H), 2.86 (s, 3H), 2.46 (s, 3H) (NH and CO₂H protons missing) |
| A130 | 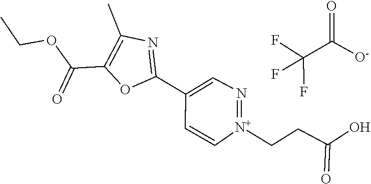 [SMILES: CCOC(=O)c1oc(-c2cc[n+](CCC(=O)O)nc2)nc1C ; O=C([O-])C(F)(F)F] | (400 MHz, D₂O) 9.94-9.89 (m, 1H), 9.89-9.84 (m, 1H), 8.97-8.92 (m, 1H), 5.09 (br t, 2H), 4.45-4.34 (m, 2H), 3.23 (br t, 2H), 2.49 (s, 3H), 1.32 (t, 3H) (CO₂H proton missing) |
| A131 | 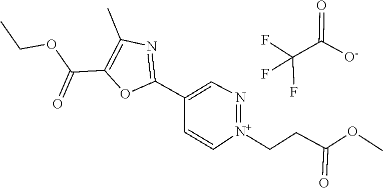 [SMILES: CCOC(=O)c1oc(-c2cc[n+](CCC(=O)OC)nc2)nc1C ; O=C([O-])C(F)(F)F] | (400 MHz, D₂O) 9.91-9.88 (m, 1H), 9.88-9.83 (m, 1H), 8.96-8.91 (m, 1H), 5.12-5.06 (m, 2H), 4.40-4.32 (m, 2H), 3.58 (s, 3H), 3.25-3.20 (m, 2H), 2.47 (s, 3H), 1.30 (t, 3H) |
| A132 | 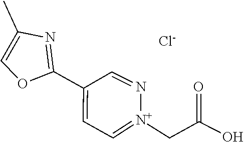 [SMILES: Cc1coc(-c2cc[n+](CC(=O)O)nc2)n1 ; [Cl-]] | (400 MHz, D₂O) 9.79 (s, 1H), 9.64 (d, 1H), 8.82 (dd, 1H), 7.95 (s, 1H), 5.54 (s, 2H), 2.21 (s, 3H) (CO₂H proton missing) |
| A133 | 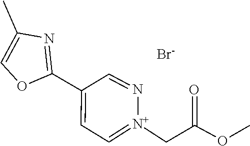 [SMILES: COC(=O)C[n+]1ccc(-c2nc(C)co2)cn1 ; [Br-]] | (400 MHz, D₂O) 9.86-9.83 (m, 1H), 9.75-9.71 (m, 1H), 8.91-8.85 (m, 1H), 8.00 (s, 1H), 5.78 (s, 2H), 3.79 (s, 3H), 2.24 (s, 3H) |
| A134 | 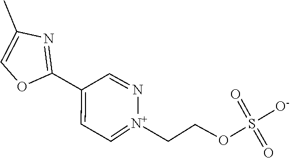 [SMILES: Cc1coc(-c2cc[n+](CCOS(=O)(=O)[O-])nc2)n1] | (400 MHz, D₂O) 9.86-9.82 (m, 1H), 9.69 (d, 1H), 8.81 (dd, 1H), 7.97 (s, 1H), 5.13-5.06 (m, 2H), 4.60-4.55 (m, 2H), 2.24 (s, 3H) |
| A135 | 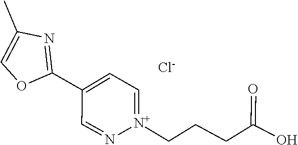 [SMILES: Cc1coc(-c2cc[n+](CCCC(=O)O)nc2)n1 ; [Cl-]] | (400 MHz, D₂O) 9.85-9.82 (m, 1H), 9.74-9.69 (m, 1H), 8.84-8.79 (m, 1H), 7.98 (s, 1H), 4.91-4.84 (m, 2H), 2.55- 2.50 (m, 2H), 2.41-2.33 (m, 2H), 2.25 (s, 3H) (CO₂H proton missing) |
| A136 | 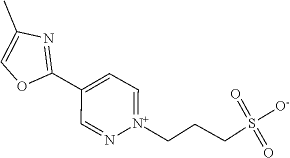 [SMILES: Cc1coc(-c2cc[n+](CCCS(=O)(=O)[O-])nc2)n1] | (400 MHz, D₂O) 9.84-9.80 (m, 1H), 9.70 (d, 1H), 8.79 (dd, 1H), 7.96 (s, 1H), 4.95 (t, 2H), 2.97 (t, 2H), 2.55- 2.45 (m, 2H), 2.23 (s, 3H) |
| A137 | 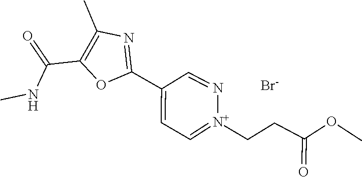 | (400 MHz, D₂O) 9.91-9.86 (m, 2H), 8.94-8.90 (m, 1H), 5.16-5.11 (m, 2H), 3.64 (s, 3H), 3.31-3.25 (m, 2H), 2.90 (s, 3H), 2.51 (s, 3H) (NH proton missing) |
| A138 | 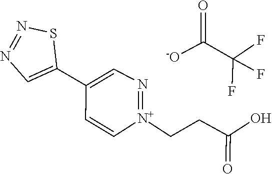 | (400 MHz, CD₃OD) 10.00 (d, 1H). 9.98 (d, 1H), 9.67 (s, 1H), 9.01 (dd, 1H), 5.13 (t, 2H), 3.25 (t, 2H) (CO₂H proton missing) |
| A139 | 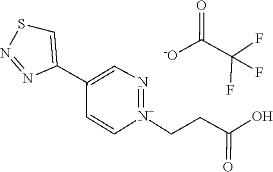 | (400 MHz, CD₃OD) 10.26 (d, 1H), 10.23 (s, 1H), 9.97 (d, 1H), 9.24 (dd, 1H), 5.13 (t, 2H), 3.27 (t, 2H) (CO₂H proton missing) |
| A140 | 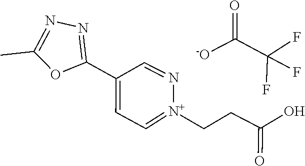 | (400 MHz, D₂O) 10.05 (d, 1H), 10.03 (d, 1H), 9.08 (dd, 1H), 5.21 (t, 2H), 3.30 (t, 2H), 2.76 (s, 3H) (CO₂H proton missing) |
| A141 | 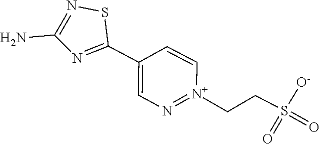 [SMILES: Nc1nsc(-c2cc[n+](CCS(=O)(=O)[O-])nc2)n1] | (400 MHz, D₂O) 9.95 (d, 1H), 9.90 (d, 1H), 8.98 (dd, 1H), 5.28 (t, 2H), 3.73 (t, 2H) (NH protons missing) |
| A142 | 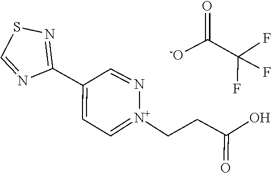 | (400 MHz, D₂O) 10.35 (s, 1H), 10.19 (d, 1H), 9.90 (d, 1H), 9.18 (dd, 1H), 5.14 (t, 2H), 3.26 (t, 2H) (CO₂H proton missing) |
| A143 | 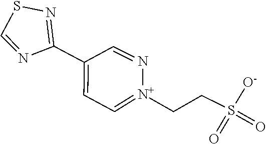 [SMILES: O=S(=O)([O-])CC[n+]1ccc(-c2ncsn2)cn1] | (400 MHz, D₂O) 10.35 (s, 1H), 10.23 (d, 1H), 9.90 (d, 1H), 9.21 (dd, 1H), 5.28 (t, 2H), 3.73 (t, 2H) |
| A144 | 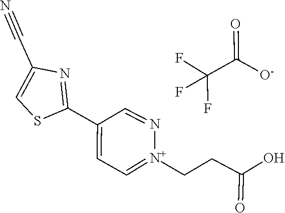 [SMILES: N#Cc1csc(-c2cc[n+](CCC(=O)O)nc2)n1 ; O=C([O-])C(F)(F)F] | (400 MHz, D₂O) 10.03 (d, 1H), 9.89 (d, 1H), 9.01-9.04 (m, 1H), 8.95 (s, 1H), 5.15 (t, 2H) 3.26 (t, 2H) (CO₂H proton missing) |
| A145 | 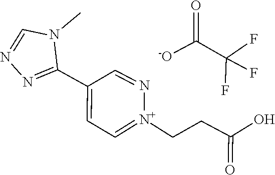 | (400 MHz, D₂O) 9.92-9.93 (m, 2H), 8.89-8.94 (m, 1H), 8.77 (s, 1H), 5.16 (t, 2H), 4.02 (s, 3H), 3.29 (t, 2H) (CO₂H proton missing) |
| A146 | 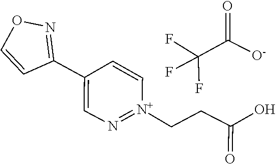 [SMILES: O=C(O)CC[n+]1ccc(-c2ccon2)cn1 ; O=C([O-])C(F)(F)F] | (400 MHz, CD₃OD) 10.06 (d, 1H), 10.03 (d, 1H), 9.12-9.02 (m, 2H), 7.42 (d, 1H), 5.15 (t, 2H), 3.27 (t, 2H) (CO₂H proton missing) |
| A147 | 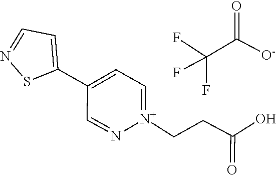 [SMILES: O=C(O)CC[n+]1ccc(-c2ccns2)cn1 ; O=C([O-])C(F)(F)F] | (400 MHz, D₂O) 9.65-9.73 (m, 2H), 8.69-8.74 (m, 1H), 8.62-8.67 (m, 1H), 8.04-8.11 (m, 1H), 4.96-5.05 (m, 2H), 3.16-3.22 (m, 2H) (CO₂H proton missing) |

### BIOLOGICAL EXAMPLES

### Post-Emergence Efficacy

Seeds of a variety of test species were sown in standard soil in pots. After cultivation for 14 days (post-emergence) under controlled conditions in a glasshouse (at 24/16° C. day/night; 14 hours light; 65% humidity), the plants were sprayed with an aqueous spray solution derived from the dissolution of the technical active ingredient formula (I) in a small amount of acetone and a special solvent and emulsifier mixture referred to as IF50 (11.12% Emulsogen EL360 TM+44.44% N-methyl pyrrolidone+44.44% Dowanol DPM glycolether), to create a 50 g/l solution which was then diluted to required concentration using 0.25% or 1% Empicol ESC₇₀ (Sodium lauryl ether sulphate)+1% ammonium sulphate as diluent. The test plants were then grown in a glasshouse under controlled conditions (at 24/16° C., day/night; 14 hours light; 65% humidity) and watered twice daily. After 13 days the test was evaluated (100=total damage to plant; 0=no damage to plant).

The results are shown in Table B (below). A value of n/a indicates that this combination of weed and test compound was not tested/assessed.

### Test Plants:

Ipomoea hederacea (IPOHE), Euphorbia heterophylla (EPHHL), Chenopodium album (CHEAL), Amaranthus palmeri (AMAPA), Lolium perenne (LOLPE), Digitaria sanguinalis (DIGSA), Eleusine indica (ELEIN), Echinochloa crus-galli (ECHCG), Setaia fabei (SETFA)

TABLE B
Control of weed species by compounds of formula
(I) after post-emergence application

| Compound | Application | | | | | | | | | |
|---|---|---|---|---|---|---|---|---|---|---|
| Number | Rate g/Ha | AMAPA | CHEAL | EPHHL | IPOHE | ELEIN | LOLPE | DIGSA | SETFA | ECHCG |
| A1 | 500 | 100 | 70 | 70 | 50 | 50 | 90 | 100 | 50 | 60 |
| A2 | 125 | 0 | 0 | 30 | 10 | 10 | 0 | 20 | 20 | 10 |
| A3 | 125 | 20 | 60 | 70 | 40 | 50 | 20 | 80 | 80 | 70 |
| A4 | 500 | 10 | 30 | 40 | 10 | 10 | 0 | 30 | 20 | 20 |
| A5 | 500 | 100 | 100 | 100 | 70 | 80 | 100 | 60 | 60 | 70 |
| A6 | 500 | 90 | 40 | 20 | 10 | 60 | 60 | 60 | 70 | 60 |
| A7 (tested as | 500 | 0 | 0 | 20 | 10 | 20 | 10 | 30 | 10 | 10 |
| a 1:1 | | | | | | | | | | |
| regioisomeric | | | | | | | | | | |
| mixture) | | | | | | | | | | |
| A8 (tested as | 500 | 10 | 10 | 30 | 30 | 20 | 10 | 50 | 40 | 50 |
| a 1:1 | | | | | | | | | | |
| regioisomeric | | | | | | | | | | |
| mixture) | | | | | | | | | | |
| A9 | 500 | 90 | 70 | 90 | 80 | 80 | 30 | 70 | 60 | 60 |
| A10 | 500 | 60 | 80 | 60 | 50 | 60 | 60 | 50 | 60 | 30 |
| A11 | 500 | 100 | 90 | 100 | 60 | 100 | 80 | 90 | 100 | 100 |
| A12 | 500 | 60 | 30 | 20 | 20 | 30 | 70 | 60 | 40 | 30 |
| A13 | 500 | 90 | 70 | 90 | 40 | 80 | 60 | 30 | 60 | 40 |
| A14 | 500 | 100 | 40 | 50 | 40 | 70 | 40 | 50 | 50 | 40 |
| A15 | 500 | 100 | 80 | 100 | 30 | 100 | 60 | 80 | 70 | 80 |
| A16 | 500 | 100 | 100 | 90 | 80 | 80 | 90 | 70 | 80 | 80 |
| A17 | 500 | 30 | 30 | 40 | 30 | 60 | 50 | 50 | 50 | 40 |
| A18 | 500 | 90 | 10 | 50 | 30 | 30 | 40 | 30 | 40 | 30 |
| A19 | 500 | 40 | 100 | 100 | 70 | 100 | 90 | 70 | 60 | 80 |
| A20 | 500 | 40 | 20 | 10 | 10 | 50 | 10 | 60 | 20 | 30 |
| A21 | 500 | 10 | 40 | 40 | 10 | 10 | 0 | 10 | 10 | 10 |
| A22 | 500 | 30 | 20 | 30 | 10 | 20 | 10 | 20 | 20 | 20 |
| A23 | 500 | 50 | 10 | 70 | 30 | 60 | 40 | 50 | 40 | 30 |
| A24 | 500 | 0 | 0 | n/a | 10 | 20 | 10 | 10 | 20 | 30 |
| A25 | 500 | 80 | 70 | 80 | 40 | 40 | 20 | 40 | 40 | 30 |
| A26 | 500 | 20 | 40 | n/a | 40 | 40 | 30 | 30 | 40 | 20 |
| A27 | 500 | 100 | 90 | 90 | 70 | 50 | 30 | 90 | 90 | 90 |
| A28 | 500 | 100 | 90 | n/a | 30 | 100 | 70 | 70 | 100 | 100 |
| A29 | 500 | 20 | 50 | 20 | 20 | 30 | 20 | 30 | 20 | 20 |
| A30 | 500 | 80 | 40 | 40 | 20 | 40 | 50 | 30 | 40 | 20 |
| A31 | 500 | 30 | 20 | 30 | 10 | 50 | 20 | 50 | 60 | 40 |
| A32 | 500 | 80 | 90 | 80 | 60 | 90 | 40 | 90 | 80 | 90 |
| A33 | 500 | 100 | 70 | 80 | 10 | 50 | 30 | 70 | 30 | 20 |
| A34 | 500 | 60 | 20 | 30 | 20 | 20 | 20 | 30 | 40 | 50 |
| A35 | 500 | 100 | 100 | 90 | 50 | 70 | 70 | 50 | 60 | 50 |
| A36 | 500 | 100 | 60 | 30 | 30 | 20 | 20 | 10 | 10 | 0 |
| A37 | 500 | 30 | 50 | 30 | 20 | 10 | 0 | 10 | 10 | 10 |
| A38 | 500 | 20 | 30 | 20 | 20 | 0 | 10 | 20 | 10 | 10 |
| A39 | 500 | 90 | 90 | 90 | 80 | 80 | 70 | 90 | 90 | 80 |
| A40 | 500 | 100 | 100 | 100 | 90 | 100 | 80 | 80 | 80 | 80 |
| A41 | 500 | 0 | 60 | n/a | 10 | 0 | 0 | 0 | 0 | 0 |
| A42 | 500 | 20 | 40 | 30 | 20 | 20 | 0 | 20 | 10 | 10 |
| A43 | 500 | 100 | 90 | 80 | 20 | 10 | 0 | 20 | 10 | 10 |
| A44 | 500 | 100 | n/a | n/a | 60 | 50 | 30 | 50 | 60 | 30 |
| A45 | 500 | 100 | 80 | 90 | 20 | 70 | 70 | 50 | 70 | 40 |
| A46 | 500 | n/a | 70 | n/a | 50 | 60 | 40 | 60 | 60 | 80 |
| A47 | 500 | 100 | 100 | 100 | 30 | 100 | 100 | 90 | 90 | 90 |
| A48 | 500 | 100 | 90 | 90 | 90 | 70 | 40 | 100 | 90 | 70 |
| A49 | 500 | 100 | 100 | 90 | 70 | 60 | 40 | 70 | 60 | 60 |
| A50 | 500 | 100 | 100 | 100 | 70 | 70 | 80 | 100 | 100 | 100 |
| A51 | 500 | 100 | 100 | 90 | 30 | 70 | 60 | 90 | 90 | 90 |
| A52 | 500 | 50 | n/a | n/a | 10 | 10 | 0 | 30 | 10 | 10 |
| A53 | 500 | 60 | n/a | n/a | 50 | 60 | 20 | 90 | 40 | 30 |
| A54 | 500 | 80 | 50 | n/a | 10 | 80 | 60 | 90 | 70 | 70 |
| A55 | 500 | 100 | 80 | n/a | 20 | 100 | 70 | 100 | 100 | 100 |
| A56 | 500 | 100 | 80 | 90 | 30 | 80 | 50 | 70 | 80 | 90 |
| A57 | 500 | 100 | 100 | 90 | 60 | 60 | 50 | 70 | 60 | 80 |
| A58 | 500 | 80 | 50 | n/a | 20 | 40 | 30 | 90 | 90 | 70 |
| A59 | 500 | 90 | 90 | 100 | 80 | 90 | 70 | 70 | 80 | 70 |
| A60 | 500 | 90 | 90 | 50 | 10 | 70 | 30 | 50 | 50 | 40 |
| A61 | 500 | 100 | 70 | 40 | 20 | 60 | 30 | 80 | 70 | 50 |
| A62 | 500 | 0 | 20 | 40 | 20 | 0 | 0 | 10 | 0 | 0 |
| A63 | 500 | 0 | 30 | 40 | 20 | 20 | 10 | 20 | 30 | 20 |
| A64 | 500 | 100 | 90 | 100 | 90 | 30 | 40 | 90 | 80 | 80 |
| A65 | 500 | 80 | 70 | 90 | 60 | 100 | 90 | 100 | 100 | 60 |
| A66 | 500 | 50 | 60 | n/a | 10 | 90 | 20 | 60 | 70 | 70 |
| A67 | 500 | 100 | 80 | 80 | 80 | 90 | 60 | 90 | 80 | 70 |
| A68 | 500 | 100 | 90 | 90 | 50 | 70 | 70 | 70 | 80 | 60 |
| A69 | 500 | 90 | 90 | n/a | 40 | 90 | 30 | 90 | 90 | 80 |
| A70 | 500 | 40 | 40 | n/a | 30 | 40 | 20 | 80 | 40 | 40 |
| A71 | 500 | 100 | 90 | 90 | 60 | 100 | 80 | 90 | 90 | 100 |
| A72 | 500 | 90 | 70 | 70 | 70 | 80 | 40 | 60 | 60 | 40 |
| A73 | 500 | 80 | 70 | 50 | 30 | 70 | 10 | 70 | 60 | 50 |
| A74 | 500 | 100 | 90 | 70 | 80 | 100 | 60 | 90 | 80 | 40 |
| A75 | 125 | 0 | 40 | n/a | 0 | 0 | 0 | 10 | 0 | 0 |
| A76 | 500 | 30 | 20 | 40 | 30 | 70 | 30 | 60 | 40 | 30 |
| A77 | 500 | 50 | 70 | n/a | 60 | 0 | 0 | 90 | 80 | 60 |
| A78 | 500 | 70 | 80 | n/a | 50 | 0 | 10 | 60 | 50 | 20 |
| A79 | 500 | 100 | 90 | n/a | 80 | 100 | 70 | 90 | 90 | 80 |
| A80 | 500 | 40 | 10 | n/a | 10 | 10 | 0 | 60 | 40 | 0 |
| A81 | 500 | 50 | 40 | n/a | 10 | 10 | 10 | 70 | 50 | 30 |
| A83 | 500 | 100 | 70 | 70 | 40 | 70 | 50 | 60 | 60 | 60 |
| A84 | 500 | 100 | 90 | 90 | 40 | 70 | 50 | 70 | 50 | 70 |
| A85 | 500 | 10 | 0 | 20 | 30 | 30 | 10 | 40 | 20 | 20 |
| A86 | 500 | 40 | 30 | 30 | 40 | 20 | 20 | 30 | 20 | 10 |
| A87 | 500 | 80 | 20 | 80 | 10 | 80 | 0 | 90 | 40 | 50 |
| A88 | 500 | 100 | 100 | 50 | 30 | 30 | 30 | 70 | 60 | 80 |
| A89 | 500 | 100 | 90 | 100 | 30 | 70 | 60 | 90 | 60 | 60 |
| A90 | 500 | 20 | 20 | 20 | 10 | 10 | 0 | 10 | 10 | 10 |
| A91 | 500 | 40 | 60 | n/a | 20 | 40 | 10 | 40 | 20 | 20 |
| A92 | 500 | 10 | 10 | 20 | 10 | 10 | 0 | 20 | 0 | 0 |
| A93 | 500 | 0 | 0 | 10 | 20 | 30 | 10 | 50 | 40 | 30 |
| A94 | 500 | 90 | 90 | 100 | 90 | 100 | 40 | 100 | 90 | 90 |
| A95 | 500 | 10 | 0 | 30 | 10 | 10 | 0 | 30 | 20 | 10 |
| A96 | 500 | 100 | 90 | 100 | 80 | 80 | 70 | 40 | 80 | 30 |
| A97 | 500 | 100 | 80 | 50 | 20 | 70 | 30 | 50 | 40 | 60 |
| A98 | 500 | 100 | 90 | 90 | 40 | 80 | 80 | 90 | 100 | 70 |
| A99 | 500 | 30 | 90 | 90 | 40 | 40 | 60 | 60 | 70 | 20 |
| A100 | 500 | 0 | 0 | 30 | 20 | 30 | 10 | 40 | 30 | 30 |
| A101 | 500 | 70 | 60 | 70 | 40 | 30 | 20 | 70 | 60 | 40 |
| A102 | 500 | 100 | 90 | 90 | 60 | 30 | 10 | 60 | 60 | 70 |
| A103 | 500 | 70 | 40 | 70 | 40 | 60 | 30 | 90 | 80 | 70 |
| A104 | 500 | 40 | 20 | 30 | 10 | 20 | 10 | 50 | 60 | 50 |
| A105 | 500 | 90 | 90 | 100 | 70 | 90 | 70 | 100 | 90 | 80 |
| A106 | 500 | 10 | 0 | 60 | 20 | 20 | 0 | 80 | 50 | 40 |
| A107 | 500 | 100 | 50 | 30 | 20 | 30 | 20 | 70 | 50 | 40 |
| A108 | 500 | 20 | 20 | 30 | 30 | 20 | 10 | 60 | 30 | 30 |
| A109 | 500 | 90 | 90 | 60 | 0 | n/a | 10 | n/a | 90 | n/a |
| A110 | 500 | 90 | 80 | 40 | 20 | 100 | 20 | 90 | 80 | 100 |
| A111 | 500 | 90 | 90 | 90 | 70 | 100 | 90 | 100 | 100 | 100 |
| A112 | 500 | 50 | 60 | 50 | 20 | 90 | 30 | 90 | 60 | 50 |
| A113 | 500 | 100 | 60 | 70 | 50 | 70 | 60 | 70 | 70 | 70 |
| A114 | 500 | 100 | 60 | n/a | 10 | 90 | 30 | 70 | 50 | 40 |
| A115 | 500 | 100 | 70 | 100 | 20 | 100 | 60 | 90 | 100 | 90 |
| A116 | 500 | 100 | 40 | 70 | 20 | 80 | 80 | 60 | 60 | 60 |
| A117 | 500 | 40 | 20 | 70 | 30 | 20 | 40 | 80 | 90 | 70 |
| A118 | 500 | 50 | 30 | 70 | 30 | 70 | 40 | 90 | 90 | 90 |
| A119 | 500 | 100 | 50 | 80 | 30 | 90 | 30 | 90 | 100 | 80 |
| A120 | 500 | 100 | 50 | 70 | 10 | 90 | 0 | 80 | 60 | 70 |
| A121 | 500 | 30 | 10 | 40 | 10 | 50 | 10 | 60 | 40 | 100 |
| A122 | 500 | 90 | 0 | 70 | 30 | 70 | 20 | 90 | 50 | 30 |
| A123 | 500 | 0 | 0 | 30 | 10 | 20 | 10 | 20 | 10 | 10 |
| A124 | 500 | 90 | 30 | 90 | 40 | 80 | 40 | 90 | 70 | 40 |
| A125 | 500 | 30 | 10 | 40 | 10 | 40 | 40 | 60 | 70 | 60 |
| A126 | 500 | 90 | 70 | 70 | 30 | 90 | 60 | 90 | 70 | 60 |
| A127 | 500 | 100 | 100 | 70 | 60 | 70 | 20 | 90 | 70 | 80 |
| A128 | 500 | 90 | 20 | 70 | 20 | 100 | 60 | 90 | 90 | 80 |
| A129 | 500 | 100 | 70 | 90 | 80 | 90 | 60 | 70 | 100 | 60 |
| A130 | 500 | 100 | 50 | 100 | 50 | 80 | 50 | 60 | 30 | 60 |
| A131 | 500 | 10 | 0 | 10 | 10 | 10 | 10 | 30 | 20 | 20 |
| A132 | 500 | 90 | 70 | 70 | 10 | 90 | 50 | 90 | 80 | 90 |
| A133 | 500 | 0 | 0 | 0 | 10 | 10 | 0 | 20 | 0 | 10 |
| A134 | 500 | 90 | 30 | 70 | 50 | 80 | 40 | 60 | 70 | 70 |
| A135 | 500 | 90 | 20 | 90 | 10 | 80 | 40 | 90 | 90 | 90 |
| A136 | 500 | 70 | 30 | 70 | 20 | 60 | 60 | 70 | 80 | 40 |
| A137 | 500 | 20 | 10 | 20 | 10 | 30 | 10 | 50 | 30 | 40 |
| A138 | 500 | 90 | 40 | 100 | 70 | 70 | 0 | 60 | 30 | 40 |
| A139 | 500 | 80 | 70 | 100 | 60 | 70 | 10 | 70 | 60 | 60 |
| A140 | 500 | 10 | 0 | 0 | 0 | 90 | 10 | 70 | 70 | 20 |
| A141 | 500 | 100 | 100 | 100 | 100 | 10 | 30 | 10 | 50 | 30 |
| A142 | 500 | 100 | 90 | 100 | 30 | 100 | 90 | 100 | 90 | 100 |
| A143 | 500 | 100 | 90 | 100 | 60 | 100 | 100 | 100 | 90 | 100 |

1. A compound of formula (I) or an agronomically acceptable salt or zwitterionic species thereof: 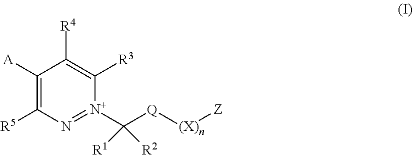
wherein
R¹ is selected from the group consisting of hydrogen, halogen, C₁-C₃alkyl, C₂-C₆alkenyl, C₂-C₆alkynyl, C₃-C₆cycloalkyl, C₁-C₆haloalkyl, —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵;
R² is selected from the group consisting of hydrogen, halogen, C₁-C₆alkyl and C₁-C₆haloalkyl;
and wherein when R¹ is selected from the group consisting of —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵, R² is selected from the group consisting of hydrogen and C₁-C₆alkyl; or
R¹ and R² together with the carbon atom to which they are attached form a C₃-C₆cycloalkyl ring or a 3- to 6-membered heterocyclyl, which comprises 1 or 2 heteroatoms individually selected from N and O; and
Q is (CR^{1a}R^{2b})ₘ;
m is 0, 1, 2 or 3;
each R^{1a} and R^{2b} are independently selected from the group consisting of hydrogen, halogen, C₁-C₆alkyl, C₁-C₆haloalkyl, —OH, —OR⁷, —OR⁵a —NH₂, —NHR⁷, —NHR^{15a}, —N(R⁶)CHO, —NR^{7b}R^{7c} and —S(O)ᵣR¹⁵; or
each R^{1a} and R^{2b} together with the carbon atom to which they are attached form a C₃-C₆cycloalkyl ring or a 3- to 6-membered heterocyclyl, which comprises 1 or 2 heteroatoms individually selected from N and O; and
R³, R⁴ and R⁵ are independently selected from the group consisting of hydrogen, halogen, cyano, nitro, —S(O)ᵣR¹⁵, C₁-C₃alkyl, C₁-C₆fluoroalkyl, C₁-C₆fluoroalkoxy, C₁-C₆alkoxy, C₃-C₆cycloalkyl and —N(R⁶)₂;
each R⁶ is independently selected from hydrogen and C₁-C₃alkyl;
each R⁷ is independently selected from the group consisting of C₁-C₃alkyl, —S(O)₂R¹⁵, —C(O)R¹⁵, —C(O)OR¹⁵ and —C(O)NR¹⁶R¹⁷;
each R^{7a} is independently selected from the group consisting of —S(O)₂R¹⁵, —C(O)R¹⁵, —C(O)OR¹⁵, —C(O)NR¹⁶R¹⁷ and —C(O)NR⁶R^{15a};
R^{7b} and R^{7c} are independently selected from the group consisting of C₁-C₃alkyl, —S(O)₂R¹⁵, —C(O)R¹⁵, —C(O)OR¹⁵, —C(O)NR¹⁶R¹⁷ and phenyl, and wherein said phenyl is optionally substituted by 1, 2 or 3 R⁹ substituents, which may be the same or different; or
R^{7b} and R^{7c} together with the nitrogen atom to which they are attached form a 4- to 6-membered heterocyclyl ring which optionally comprises one additional heteroatom individually selected from N, O and S; and
A is a 5-membered heteroaryl attached to the rest of the molecule via a ring carbon atom, which comprises 1, 2, 3 or 4 heteroatoms independently selected from the group consisting of N, O and S, and wherein the heteroaryl is optionally substituted by 1, 2 or 3 R⁸ substituents, which may be the same or different,
and wherein when A is substituted on one or more ring carbon atoms, each R⁸ is independently selected from the group consisting of halogen, nitro, cyano, —NH₂, —NHR⁷, —N(R⁷)₂, —OH, —OR⁷, —S(O)ᵣR¹⁵, —NR⁶S(O)₂R¹⁵, —C(O)OR¹⁰, —C(O)R¹⁵, —C(O)NR¹⁶R¹⁷, —S(O)₂NR¹⁶R¹⁷, C₁-C₃alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₃alkoxyC₁-C₃alkoxy-, C₁-C₆haloalkoxy, C₁-C₃haloalkoxyC₁-C₃alkyl-, C₃-C₆alkenyloxy, C₃-C₆alkynyloxy, N—C₃-C₆cycloalkylamino, —C(R⁶)═NOR⁶, phenyl, a 3- to 6-membered heterocyclyl, which comprises 1 or 2 heteroatoms individually selected from N and O, and a 5- or 6-membered heteroaryl, which comprises 1, 2, 3 or 4 heteroatoms independently selected from N, O and S, and wherein said phenyl, heterocyclyl or heteroaryl are optionally substituted by 1, 2 or 3 R⁹ substituents, which may be the same or different;
and/or
when A is substituted on a ring nitrogen atom, R⁸ is selected from the group consisting of —OR⁷, C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₃alkoxyC₁-C₃alkoxy-, C₁-C₆haloalkoxy, C₁-C₃haloalkoxyC₁-C₃alkyl-, C₃-C₆alkenyloxy and C₃-C₆alkynyloxy; and
each R⁹ is independently selected from the group consisting of halogen, cyano, —OH, —N(R⁶)₂, C₁-C₄alkyl, C₁-C₄alkoxy, C₁-C₄haloalkyl and C₁-C₄haloalkoxy;
X is independently selected from the group consisting of C₃-C₆cycloalkyl, phenyl, a 5- or 6-membered heteroaryl, which comprises 1, 2, 3 or 4 heteroatoms independently selected from N, O and S, and a 4- to 6-membered heterocyclyl, which comprises 1, 2 or 3 heteroatoms independently selected from N, O and S, and wherein said cycloalkyl, phenyl, heteroaryl or heterocyclyl moieties are optionally substituted by 1 or 2 R⁹ substituents, and wherein the aforementioned CR¹R² and Z, or Q and Z, moieties may be attached at any position of said cycloalkyl, phenyl, heteroaryl or heterocyclyl moieties;
n is 0 or 1;
Z is selected from the group consisting of —C(O)OR¹⁰, —CH₂OH, —CHO, —C(O)NHOR¹¹, —C(O)NHCN, —OC(O)NHOR¹¹, —OC(O)NHCN, —NR⁶C(O)NHOR¹¹, —NR⁶C(O)NHCN, —C(O)NHS(O)₂R¹², —OC(O)NHS(O)₂R¹², —NR⁶C(O)NHS(O)₂R¹², —S(O)₂OR⁰, —OS(O)₂OR¹⁰, —NR⁶S(O)₂OR¹⁰, —NR⁶S(O)OR¹⁰, —NHS(O)₂R¹⁴, —S(O)OR¹⁰, —OS(O)OR¹⁰, —S(O)₂NHCN, —S(O)₂NHC(O)R¹⁸, —S(O)₂NHS(O)₂R¹², —OS(O)₂NHCN, —OS(O)₂NHS(O)₂R¹², —OS(O)₂NHC(O)R¹⁸, —NR⁶S(O)₂NHCN, —NR⁶S(O)₂NHC(O)R¹⁸, —N(OH)C(O)R¹⁵, —ONHC(O)R¹⁵, —NR⁶S(O)₂NHS(O)₂R¹², —P(O)(R¹³)(OR¹⁰), —P(O)H(OR¹⁰), —OP(O)(R¹³)(OR¹⁰), —NR⁶P(O)(R¹³)(OR¹⁰) and tetrazole;
R¹⁰ is selected from the group consisting of hydrogen, C₁-C₆alkyl, phenyl and benzyl, and wherein said phenyl or benzyl are optionally substituted by 1, 2 or 3 R⁹ substituents, which may be the same or different;
R¹¹ is selected from the group consisting of hydrogen, C₁-C₆alkyl and phenyl, and wherein said phenyl is optionally substituted by 1, 2 or 3 R⁹ substituents, which may be the same or different;
R¹² is selected from the group consisting of C₁-C₆alkyl, C₁-C₆haloalkyl, C₁-C₆alkoxy, —OH, —N(R⁶)₂ and phenyl, and wherein said phenyl is optionally substituted by 1, 2 or 3 R⁹ substituents, which may be the same or different;
R¹³ is selected from the group consisting of —OH, C₁-C₆alkyl, C₁-C₆alkoxy and phenyl;
R¹⁴ is C₁-C₆haloalkyl;
R¹⁵ is selected from the group consisting of C₁-C₆alkyl and phenyl, and wherein said phenyl is optionally substituted by 1, 2 or 3 R⁹ substituents, which may be the same or different;
R^{15a} is phenyl, wherein said phenyl is optionally substituted by 1, 2 or 3 R⁹ substituents, which may be the same or different;
R¹⁶ and R¹⁷ are independently selected from the group consisting of hydrogen and C₁-C₆alkyl; or
R¹⁶ and R¹⁷ together with the nitrogen atom to which they are attached form a 4- to 6-membered heterocyclyl ring which optionally comprises one additional heteroatom independently selected from N, O and S; and
R¹⁸ is selected from the group consisting of hydrogen, C₁-C₆alkyl, C₁-C₆haloalkyl, C₁-C₆alkoxy, —N(R⁶)₂ and phenyl, and wherein said phenyl is optionally substituted by 1, 2 or 3 R⁹ substituents, which may be the same or different;
and
r is 0, 1 or 2.

2. The compound according to claim 1, wherein R¹ and R² are each independently hydrogen or C₁-C₆alkyl.

3. The compound according to claim 1, wherein each R^{1a} and R^{2b} are independently selected from the group consisting of hydrogen, C₁-C₆alkyl, —OH and —NH₂.

4. The compound according to claim 1, wherein m is 1 or 2.

5. The compound according to claim 1, wherein R³, R⁴ and R⁵ are hydrogen.

6. The compound according to claim 1, wherein A is a heteroaryl selected from the group consisting of tetrazolyl, 1,2,4-triazolyl, isoxazolyl, oxazolyl, thiazolyl, 1,3,4-thiadiazolyl, 1,2,3-triazolyl, pyrazolyl, 1,3,4-oxadiazolyl, 1,2,4-thiadiazolyl, imidazolyl, isothiazolyl, thienyl, furyl, 1,2,4-oxadiazolyl, 1,2,3-thiadiazolyl and 1,2,5-thiadiazolyl, wherein the heteroaryl is optionally substituted by 1, 2 or 3 R⁸ substituents, which may be the same or different and R⁸ is as defined in claim 1.

7. The compound according to claim 1, wherein A is selected from the group consisting of formula A-I to A-XXXIV below [chemical structure]
wherein the jagged line defines the point of attachment to the remaining part of a compound of formula (I);
R^{8a} is selected from the group consisting of hydrogen, C₁-C₆alkyl and C₁-C₆haloalkyl;
each R^{8b}, R^{8c} and R^{8d} are independently selected from the group consisting of hydrogen, halogen, nitro, cyano, —NH₂, —NHR⁷, —N(R⁷)₂, —OH, —OR⁷, —S(O)ᵣR¹⁵, —NR⁶S(O)₂R¹⁵, —C(O)OR¹⁰, —C(O)R¹⁵, —C(O)NR¹⁶R¹⁷, —S(O)₂NR¹⁶R¹⁷, C₁-C₆alkyl and C₁-C₆haloalkyl;
and R⁶, R⁷, R¹⁰, R¹⁵, R¹⁶, R¹⁷ and r are as defined in claim 1.

8. The compound according to claim 1, wherein A is selected from the group consisting of formula A-I to A-VIII, A-X, A-XIV, A-XVIII, A-XXVII, A-XXIX and A-XXX below 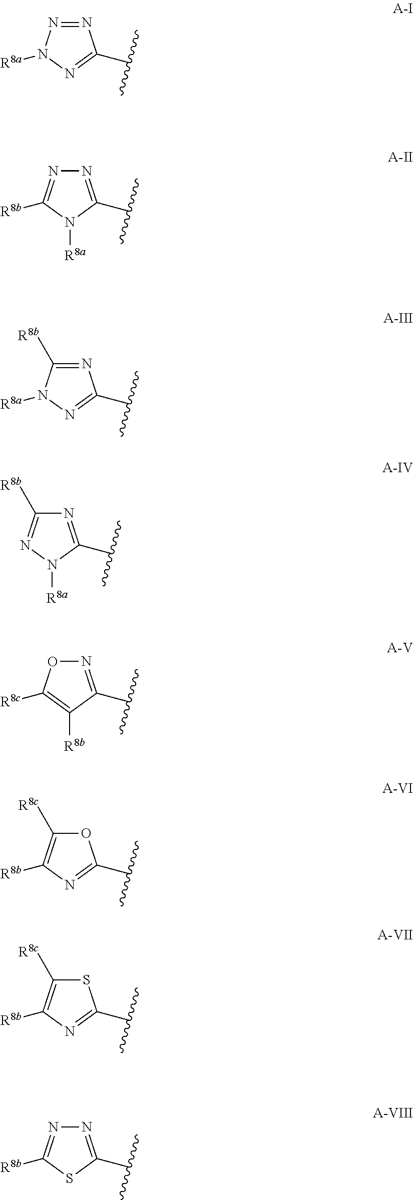 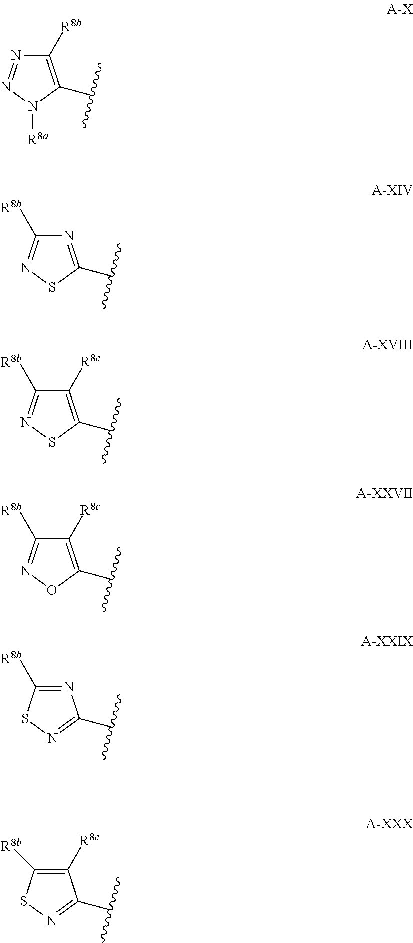
wherein the jagged line defines the point of attachment to the remaining part of a compound of formula (I);
R^{8a} is hydrogen or C₁-C₆alkyl;
each R^{8b}, R^{8c} and R^{8d} are independently selected from the group consisting of hydrogen, halogen, cyano, —NH₂, —NHR⁷, —N(R⁷)₂, —OH, —OR⁷, —S(O)ᵣR¹⁵, —C(O)OR¹⁰, —C(O)R¹⁵, —C(O)NR¹⁶R¹⁷, C₁-C₆alkyl and C₁-C₆haloalkyl;
and R⁷, R¹⁰, R¹⁵, R¹⁶, R¹⁷ and r as defined in claim 1.

9. The compound according to claim 8, wherein R^{8a} is hydrogen or methyl and each R^{8b}, R^{8c} and R^{8d} are independently selected from the group consisting of hydrogen, chloro, cyano, —NH₂, —NHMe, —OMe, —C(O)OEt, —C(O)NHMe, methyl, iso-propyl and trifluoromethyl.

10. The compound according to claim 1, wherein A is selected from the group consisting of formula A-Ia, A-IIa, A-IIIa, A-IVa, A-Va, A-VIa, A-VIb, A-VIc, A-VIIa, A-VIIb, A-VIIIa, A-VIIIb, A-Xa, A-XIVa, A-XVIIIa, A-XVIIIb, A-XXVIIa, A-XXIXa and A-XXXa below

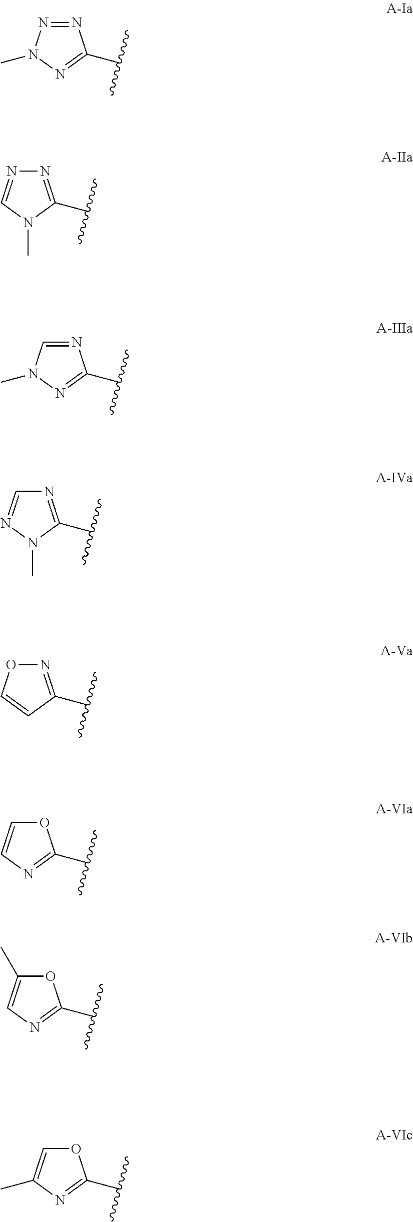

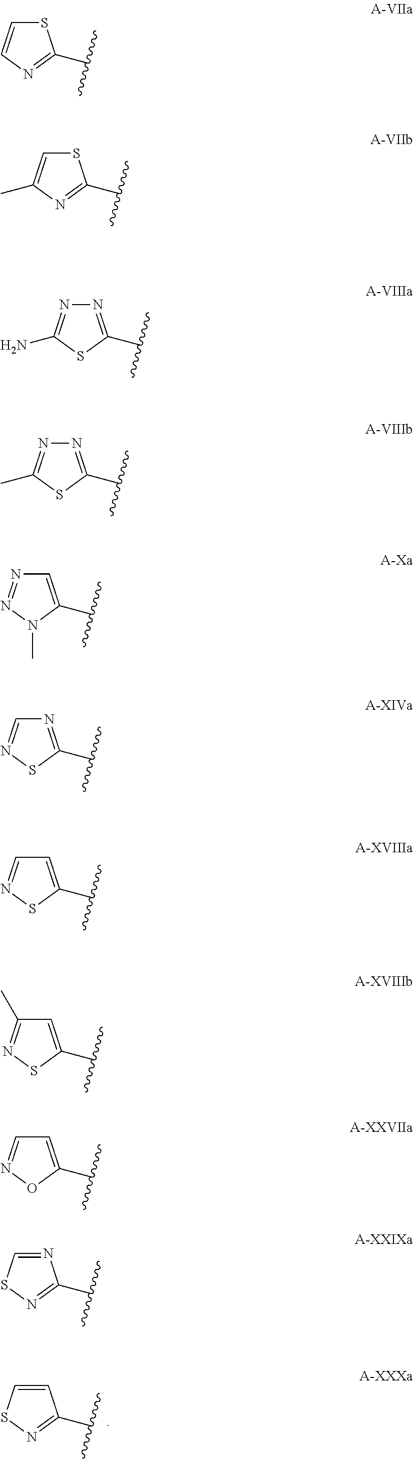

11. The compound according to claim 1, wherein Z is selected from the group consisting of —C(O)OR¹⁰, —CH₂OH, —C(O)NHS(O)₂R¹², —S(O)₂OR¹⁰, —OS(O)₂OR¹⁰, —NR⁶S(O)₂OR¹⁰ and —P(O)(R¹³)(OR¹⁰).

12. The compound according to claim 1, wherein Z is —C(O)OH or —S(O)₂OH.

13. A herbicidal composition comprising a herbicidally effective amount of a compound of formula (I) as defined in claim 1 and an agrochemically-acceptable diluent or carrier.

14. The composition according to claim 13, further comprising at least one additional active ingredient.

15. A method of controlling unwanted plant growth, comprising applying a compound of formula (I) as defined in claim 1 to the unwanted plants or to the locus thereof.

16. A method of controlling unwanted plant growth, comprising applying a herbicidal composition according to claim 13, to the unwanted plants or to the locus thereof.
